(19) United States
(12) Patent Application Publication
(10) Pub. No.: US 2024/0365739 A1
(43) Pub. Date: Nov. 7, 2024

(54) COLD TOLERANT BASIL VARIETIES

(71) Applicant: Rutgers, The State University of New Jersey, New Brunswick, NJ (US)

(72) Inventors: James E. Simon, Princeton, NJ (US); Lara J. Brindisi, Monroe, NJ (US); Christain A. Wyenandt, Elmer, NJ (US)

(73) Assignee: Rutgers, The State University of New Jersey, New Brunswick, NJ (US)

(21) Appl. No.: 18/654,839

(22) Filed: May 3, 2024

Related U.S. Application Data
(60) Provisional application No. 63/500,208, filed on May 4, 2023.

(51) Int. Cl. A01H 6/50; A01H 5/12

(52) U.S. Cl. CPC A01H 6/506; A01H 5/12

(57) ABSTRACT

Twenty-five new basil cultivars designated ‘OCB-029’, ‘OCB-017’, ‘OCB-022’, ‘OCB-009’, ‘OCB-011’, ‘OCB-027’, ‘OCB-019’, ‘OCB-031’, ‘OCB-121’, ‘OCB-174’, ‘OCB-169’, ‘OCB-153’, ‘OCB-115’, ‘OCB-120’, ‘OCB-119’, ‘OCB-067’, ‘OCB-058’, ‘OCB-086’, ‘OCB-062’, ‘OCB-117’, ‘OCB-057’, ‘OCB-126’, ‘OCB-188’, ‘OCB-129’, and ‘OCB-051’ are provided, as well as parent ‘CB15’. Also provided are parts of the plants, extracts and biomasses from the varieties, and uses thereof, for example as a food or in a food product. These plants are chilling tolerant.

### CROSS-REFERENCE TO RELATED APPLICATIONS

This application claims priority to U.S. Provisional Application No. 63/500,208, filed May 4, 2023, which is herein incorporated by reference in its entirety.

### FIELD

The present disclosure provides new basil (Ocimum basilicum) cultivars designated ‘OCB-029’, ‘OCB-017’, ‘OCB-022’, ‘OCB-009’, ‘OCB-011’, ‘OCB-027’, ‘OCB-019’, ‘OCB-031’, ‘OCB-121’, ‘OCB-174’, ‘OCB-169’, ‘OCB-153’, ‘OCB-115’, ‘OCB-120’, ‘OCB-119’, ‘OCB-067’, ‘OCB-058’, ‘OCB-086’, ‘OCB-062’, ‘OCB-117’, ‘OCB-057’, ‘OCB-126’, ‘OCB-188’, ‘OCB-129’, and ‘OCB-051’, as well as parent ‘CB15’, parts of the plants, extracts and biomasses from these varieties, and uses thereof, for example as a food or in a food product or for ornamental applications, as well as breeding material.

### BACKGROUND

Basil (Ocimum spp.) is the most commercially important culinary herb in the U.S. and in cuisines around the world. Sweet basil is the most common type of basil grown in the US and can be marketed as a fresh, frozen or dried product [1]. It is categorized as an ultra-niche, high-value annual crop with the global basil market valued at $57 million in 2020 and expecting to reach $63 million by the end of 2027 [2]. The vast majority of sweet basil produced is marketed as fresh-cut where freshness, texture, shelf life, and aroma are crucial.

Basil (O. basilicum L.) is generally recognized as a tetraploid [3-10]. However, the total number of chromosomes has been observed to vary suggesting a complex history of polyploidy and aneuploidy [3, 5, 6, 9, 11-13]. DNA content is estimated to be 3.9-4.7 pg/2 C [9, 14], with an estimated genome size estimation of 2.13 Gb [10]. The genome is characterized as highly repetitive, mainly due to long-terminal repeats [10]. Much work has been done to understand the genetics of basil, including cytological screening [4, 7-9, 12, 13], karyotyping [3, 5, 13], flow cytometry [9, 14, 15], RAPD, AFLP and SSR analyses [9, 16], de novo linkage mapping [16], RNA-sequencing [17, 18], association mapping [19] and construction of a draft reference genome [10]. Furthermore, our team recently assembled a chromosome-level reference map for O. basilicum and will soon be releasing the corresponding genome annotation [20]. Despite these advancements, much remains to be understood about the genetics of basil, including but not limited to, the potential existence of sub-genomes and their interactions, modes of genetic inheritance and mechanisms for polyploidy and aneuploidy events.

The Ocimum genus, and the Lamiaceae or “mint” family in general, are clades with a wide diversity of highly aromatic plants. O. basilicum includes plants that not only produce the traditional sweet basil aroma, but also licorice, cinnamon and clove aromas to name just a few [21-24]. Descriptive analyses with trained panels have identified volatile compounds responsible for the key aromas in O. basilicum by correlating sensory evaluations with chemistry analysis [21, 22]. These compounds can be objectively detected and quantified via gas chromatography (GC) mass spectrometry (MS) by liquid injection with the essential oils [22, 23, 25, 26], or by headspace (HS) with dried [21, 26] or fresh plant material [24]. HS is quite useful for relative quantification due to its operational simplicity and ability to avoid hydrodistillation, which can alter the composition of essential oils [26, 27]. HS can be coupled with solid phase microextraction (SPME) to increase detection sensitivity and the number of compounds detected [26, 28] with the caveat that the SPME fiber uniformly sensitive to all compounds [29]. HS-SPME has become a common method for volatile analysis due to its ability to detect minor compounds and has been successfully used to identify aroma compounds in basil [30-32].

Major aromatic compounds produced by sweet basil and Thai basil include estragole (also known as methyl chavicol and p-allylanisole), eucalyptol (also known as 1,8-cineole), linalool and eugenol. Linalool and eucalyptol belong to the monoterpene and monoterpenoid classes, respectively. Their biosynthetic pathways differ in the composition of their monoterpene synthases, but they are both derived from the same starting compound, geranyl diphosphate produced from the mevalonate pathway [33, 34]. However, estragole is a phenylpropene derived from phenylalanine produced from the phenylpropanoid pathway and eugenol is a phenylpropanoid derived from guaiacol [35].

Basil is native to hot, humid environments with its primary center of diversity being in tropical Africa, and secondary centers in tropical Asia and tropical/subtropical South America [1, 36, 37]. Similar to many other plants with tropical origins, basil is “chilling sensitive” with the risk of temperatures less than 12° C. (54° C.) causing injury and temperatures less than 10° C. (50° F.) inducing severe if not total injury of basil leaves [38-40]. Chilling injury symptoms include leaf necrosis, which appears as leaf spotting or browning, wilting or loss of leaf turgidity, and decay [40-43]. Chilling sensitivity in basil is a major problem for distributers as it is typically shipped at low temperatures with other herbs to minimize disease and decay and costly separate shipping arrangements [41]. Other herbs tend to tolerate these low temperatures, but basil does not, which incurs large post-harvest losses [44].

There are no Genovese-style sweet basils (O. basilicum) on the commercial market found to be significantly chilling tolerant to date. Genovese represents the “gold standard” of sweet or Italian basil. The aroma is complex yet distinctly floral, spicy and clove-like. The flowers are white and the leaves are large, glossy and green with a convex cross section. The basil varieties that claim to be cold tolerant have camphor, licorice or pine aromas, with morphological characteristics referred to as “ornamental”, such as purple flowers and smaller, matte, purple-green and flat leaves. There are management strategies that help reduce chilling injury in basil [44] including harvesting in the afternoon [40, 41], acclimating plants with less severe low temperatures [39, 40], supplementing with artificial lighting [44, 45] and packaging samples in low density polyethylene bags [39, 46]. However, these approaches have limited success and growers, processors and distributors still suffer sizable economic losses with these added expenses.

### SUMMARY

The present disclosure provides a series of new basil cultivars that are cold tolerant. Over 6,000 seeds of Italian large leaf basil (O. basilicum) were screened for chilling tolerance. Chilling tolerance was hypothesized to be associated with estragole as most of the chilling basil (CB) lines released a strong licorice aroma. ‘CB15’ (unpatented) was later identified as having the most chilling tolerance of the CB lines under both laboratory and simulated commercial practices, however, further breeding efforts were needed to obtain a chilling tolerant basil with Genovese-style aroma [43]. Thus, ‘CB15’ was crossed with ‘RU Obsession DMR’ (U.S. Pat. No. 10,159,212) to impart its Genovese-style aroma on the progeny, and its downy mildew resistance/tolerance (DMR), Fusarium oxysporum f. sp. basilica (FOB) resistance, moderate leaf size and late flowering time.

Genotyping-by-sequencing (GBS) and double digestion restriction-site associated digestion sequencing (ddRADSeq) were used to construct genetic linkage maps for an F₂ mapping population from a controlled cross between ‘Rutgers Obsession DMR’ and ‘CB15’. Quantitative trait loci (QTL) related to chilling tolerance and key volatile compounds were identified and it was determined if chilling response was associated with aroma. The F₂ progeny segregated for chilling tolerance and two aroma compound concentrations, enabling QTL detection for chilling tolerance, estragole and eucalyptol.

The top 25 selections of the F2 lines resulting from the cross between ‘Rutgers Obsession DMR’ and ‘CB15’ were selected according to their sweet basil or Thai aroma types and chilling tolerance, downy mildew resistance/tolerance, Fusarium resistance, leaf morphology, flowering time, habit and value in both indoor and outdoor production. The new basil cultivars are chilling tolerant and can be used in indoor or outdoor cultivation. Some are also downy mildew resistant (DMR) and/or highly tolerant to basil downy mildew. Twenty-five new cultivars, ‘OCB-029’, ‘OCB-017’, ‘OCB-022’, ‘OCB-009’, ‘OCB-011’, ‘OCB-027’, ‘OCB-019’, ‘OCB-031’, ‘OCB-121’, ‘OCB-174’, ‘OCB-169’, ‘OCB-153’, ‘OCB-115’, ‘OCB-120’, ‘OCB-119’, ‘OCB-067’, ‘OCB-058’, ‘OCB-086’, ‘OCB-062’, ‘OCB-117’, ‘OCB-057’, ‘OCB-126’, ‘OCB-188’, ‘OCB-129’, and ‘OCB-051’, were identified as having chilling tolerance, for example the ability of their leaves to withstand exposure to refrigeration for about 4-5 days at about 3-5° C., as demonstrated by an average of less than 5% leaf necrosis. These new cultivars have the visual appearance of sweet basil and the aroma of traditional sweet or Thai basils. These new cultivars can be used for the fresh, dried, processed, fresh frozen, extracts, ornamental, perfume, flavor, supplement and heath markets for growers, processors, retailers and horticulturalists/home gardeners. In some examples, these new cultivars are used for their aroma and flavor profile, for example in perfumes, teas, and foods. In some examples, these new cultivars are used in a nutraceutical.

The present disclosure provides new basils ‘OCB-029’, ‘OCB-017’, ‘OCB-022’, ‘OCB-009’, ‘OCB-011’, ‘OCB-027’, ‘OCB-019’, ‘OCB-031’, ‘OCB-121’, ‘OCB-174’, ‘OCB-169’, ‘OCB-153’, ‘OCB-115’, ‘OCB-120’, ‘OCB-119’, ‘OCB-067’, ‘OCB-058’, ‘OCB-086’, ‘OCB-062’, ‘OCB-117’, ‘OCB-057’, ‘OCB-126’, ‘OCB-188’, ‘OCB-129’, and ‘OCB-051, as well as parent ‘CB15’, that are chilling tolerant, which are distinct from all basils in the marketplace. Also provided herein are methods of producing these new cultivars, a list of the new cultivars along with descriptors, and examples using somaclonal variations in plant tissue culture and protoplast culture conditions as well as induced mutagenesis technologies that can be used to multiply these new cultivars and inbred lines. The disclosure also provides extracts and aroma profiles of these cultivars, methods of making such extracts and procuring such aromas.

Each of the new cultivars will be deposited with the American Type Culture Collection (ATCC), 10801 University Blvd., Manassas, VA, 20110. The deposits are intended to meet all of the requirements of 37 C.F.R. §§ 1.801-1.809. Access to the deposits will be available during the pendency of the application to the Commissioner of Patents and Trademarks and persons determined by the Commissioner to be entitled thereto upon request. The deposits will be maintained in the depository for a period of 30 years, or 5 years after the last request, or for the effective life of the patent, whichever is longer, and will be replaced if necessary during that period. Applicants do not waive any infringement of rights granted under this patent or under the Plant Variety Protection Act (7 U.S.C. 2321 et seq.).

Provided herein are new basil cultivars (‘OCB-029’, ‘OCB-017’, ‘OCB-022’, ‘OCB-009’, ‘OCB-011’, ‘OCB-027’, ‘OCB-019’, ‘OCB-031’, ‘OCB-121’, ‘OCB-174’, ‘OCB-169’, ‘OCB-153’, ‘OCB-115’, ‘OCB-120’, ‘OCB-119’, ‘OCB-067’, ‘OCB-058’, ‘OCB-086’, ‘OCB-062’, ‘OCB-117’, ‘OCB-057’, ‘OCB-126’, ‘OCB-188’, ‘OCB-129’, and ‘OCB-051’), as well as parent ‘CB15’, that are chilling tolerant, as well as progeny of such plants, plant parts, including leaves, cells, plant protoplasts, plant cells of a tissue culture from which basil plants can be regenerated, plant calli, plant clumps, and plant cells that are intact in plants or parts of plants, such as pollen, ovules flowers, seeds, leaves, stems, roots and the like. Also provided is plurality of one or more of ‘OCB-029’, ‘OCB-017’, ‘OCB-022’, ‘OCB-009’, ‘OCB-011’, ‘OCB-027’, ‘OCB-019’, ‘OCB-031’, ‘OCB-121’, ‘OCB-174’, ‘OCB-169’, ‘OCB-153’, ‘OCB-115’, ‘OCB-120’, ‘OCB-119’, ‘OCB-067’, ‘OCB-058’, ‘OCB-086’, ‘OCB-062’, ‘OCB-117’, ‘OCB-057’, ‘OCB-126’, ‘OCB-188’, ‘OCB-129’, and ‘OCB-051’ basil plants, as well as parent ‘CB15’, grown in an outdoor field, or greenhouse or in specialized growth facilities, for example under controlled environmental conditions. In addition to the ‘OCB-029’, ‘OCB-017’, ‘OCB-022’, ‘OCB-009’, ‘OCB-011’, ‘OCB-027’, ‘OCB-019’, ‘OCB-031’, ‘OCB-121’, ‘OCB-174’, ‘OCB-169’, ‘OCB-153’, ‘OCB-115’, ‘OCB-120’, ‘OCB-119’, ‘OCB-067’, ‘OCB-058’, ‘OCB-086’, ‘OCB-062’, ‘OCB-117’, ‘OCB-057’, ‘OCB-126’, ‘OCB-188’, ‘OCB-129’, and ‘OCB-051’ basil plant varieties, as well as parent ‘CB15’, derivatives of such plants retaining chilling tolerance are provided. In one example, the disclosure provides basil plants having the genotype of one of the new varieties disclosed herein, for example generated through sexual reproduction. For example, the disclosure provides plants produced by growing the seed of ‘OCB-029’, ‘OCB-017’, ‘OCB-022’, ‘OCB-009’, ‘OCB-011’, ‘OCB-027’, ‘OCB-019’, ‘OCB-031’, ‘OCB-121’, ‘OCB-174’, ‘OCB-169’, ‘OCB-153’, ‘OCB-115’, ‘OCB-120’, ‘OCB-119’, ‘OCB-067’, ‘OCB-058’, ‘OCB-086’, ‘OCB-062’, ‘OCB-117’, ‘OCB-057’, ‘OCB-126’, ‘OCB-188’, ‘OCB-129’, and ‘OCB-051’, as well as parent ‘CB15’, disclosed herein.

In one example, the disclosure provides basil plants having the genotype of one of the new varieties disclosed herein, for example generated through asexual reproduction. For example, the disclosure provides plants produced by growing a cutting of ‘OCB-029’, ‘OCB-017’, ‘OCB-022’, ‘OCB-009’, ‘OCB-011’, ‘OCB-027’, ‘OCB-019’, ‘OCB-031’, ‘OCB-121’, ‘OCB-174’, ‘OCB-169’, ‘OCB-153’, ‘OCB-115’, ‘OCB-120’, ‘OCB-119’, ‘OCB-067’, ‘OCB-058’, ‘OCB-086’, ‘OCB-062’, ‘OCB-117’, ‘OCB-057’, ‘OCB-126’, ‘OCB-188’, ‘OCB-129’, and/or ‘OCB-051’. In another example, the disclosure provides plants produced by growing a cutting of ‘CB15’.

In some examples, ‘OCB-029’, ‘OCB-017’, ‘OCB-022’, ‘OCB-009’, ‘OCB-011’, ‘OCB-027’, ‘OCB-019’, ‘OCB-031’, ‘OCB-121’, ‘OCB-174’, ‘OCB-169’, ‘OCB-153’, ‘OCB-115’, ‘OCB-120’, ‘OCB-119’, ‘OCB-067’, ‘OCB-058’, ‘OCB-086’, ‘OCB-062’, ‘OCB-117’, ‘OCB-057’, ‘OCB-126’, ‘OCB-188’, ‘OCB-129’, and/or ‘OCB-051’ includes linkage group (LG) 24 marker 938232. In some examples, an F1, F2, F3, F4, F5, F6, F7, F8, F9, or F10 generation of ‘OCB-029’, ‘OCB-017’, ‘OCB-022’, ‘OCB-009’, ‘OCB-011’, ‘OCB-027’, ‘OCB-019’, ‘OCB-031’, ‘OCB-121’, ‘OCB-174’, ‘OCB-169’, ‘OCB-153’, ‘OCB-115’, ‘OCB-120’, ‘OCB-119’, ‘OCB-067’, ‘OCB-058’, ‘OCB-086’, ‘OCB-062’, ‘OCB-117’, ‘OCB-057’, ‘OCB-126’, ‘OCB-188’, ‘OCB-129’, and/or ‘OCB-051’ includes LG24 marker 938232. In some examples, progeny of ‘OCB-029’, ‘OCB-017’, ‘OCB-022’, ‘OCB-009’, ‘OCB-011’, ‘OCB-027’, ‘OCB-019’, ‘OCB-031’, ‘OCB-121’, ‘OCB-174’, ‘OCB-169’, ‘OCB-153’, ‘OCB-115’, ‘OCB-120’, ‘OCB-119’, ‘OCB-067’, ‘OCB-058’, ‘OCB-086’, ‘OCB-062’, ‘OCB-117’, ‘OCB-057’, ‘OCB-126’, ‘OCB-188’, ‘OCB-129’, and/or ‘OCB-includes LG24 marker 938232.

In some examples, ‘OCB-029’, ‘OCB-017’, ‘OCB-022’, ‘OCB-009’, ‘OCB-011’, ‘OCB-027’, ‘OCB-019’, ‘OCB-031’, ‘OCB-121’, ‘OCB-174’, ‘OCB-169’, ‘OCB-153’, ‘OCB-115’, ‘OCB-120’, ‘OCB-119’, ‘OCB-067’, ‘OCB-058’, ‘OCB-086’, ‘OCB-062’, ‘OCB-117’, ‘OCB-057’, ‘OCB-126’, ‘OCB-188’, ‘OCB-129’, and/or ‘OCB-051’ includes LG1. In some examples, an F1, F2, F3, F4, F5, F6, F7, F8, F9, or F10 generation of ‘OCB-029’, ‘OCB-017’, ‘OCB-022’, ‘OCB-009’, ‘OCB-011’, ‘OCB-027’, ‘OCB-019’, ‘OCB-031’, ‘OCB-121’, ‘OCB-174’, ‘OCB-169’, ‘OCB-153’, ‘OCB-115’, ‘OCB-120’, ‘OCB-119’, ‘OCB-067’, ‘OCB-058’, ‘OCB-086’, ‘OCB-062’, ‘OCB-117’, ‘OCB-057’, ‘OCB-126’, ‘OCB-188’, ‘OCB-129’, and/or ‘OCB-051’ includes LG1. In some examples, progeny of ‘OCB-029’, ‘OCB-017’, ‘OCB-022’, ‘OCB-009’, ‘OCB-011’, ‘OCB-027’, ‘OCB-019’, ‘OCB-031’, ‘OCB-121’, ‘OCB-174’, ‘OCB-169’, ‘OCB-153’, ‘OCB-115’, ‘OCB-120’, ‘OCB-119’, ‘OCB-067’, ‘OCB-058’, ‘OCB-086’, ‘OCB-062’, ‘OCB-117’, ‘OCB-057’, ‘OCB-126’, ‘OCB-188’, ‘OCB-129’, and/or ‘OCB-includes LG1.

In some examples, ‘OCB-029’, ‘OCB-017’, ‘OCB-022’, ‘OCB-009’, ‘OCB-011’, ‘OCB-027’, ‘OCB-019’, ‘OCB-031’, ‘OCB-121’, ‘OCB-174’, ‘OCB-169’, ‘OCB-153’, ‘OCB-115’, ‘OCB-120’, ‘OCB-119’, ‘OCB-067’, ‘OCB-058’, ‘OCB-086’, ‘OCB-062’, ‘OCB-117’, ‘OCB-057’, ‘OCB-126’, ‘OCB-188’, ‘OCB-129’, and/or ‘OCB-051’ includes LG1 marker 765. In some examples, an F1, F2, F3, F4, F5, F6, F7, F8, F9, or F10 generation of ‘OCB-029’, ‘OCB-017’, ‘OCB-022’, ‘OCB-009’, ‘OCB-011’, ‘OCB-027’, ‘OCB-019’, ‘OCB-031’, ‘OCB-121’, ‘OCB-174’, ‘OCB-169’, ‘OCB-153’, ‘OCB-115’, ‘OCB-120’, ‘OCB-119’, ‘OCB-067’, ‘OCB-058’, ‘OCB-086’, ‘OCB-062’, ‘OCB-117’, ‘OCB-057’, ‘OCB-126’, ‘OCB-188’, ‘OCB-129’, and/or ‘OCB-051’ includes LG1 marker 765. In some examples, progeny of ‘OCB-029’, ‘OCB-017’, ‘OCB-022’, ‘OCB-009’, ‘OCB-011’, ‘OCB-027’, ‘OCB-019’, ‘OCB-031’, ‘OCB-121’, ‘OCB-174’, ‘OCB-169’, ‘OCB-153’, ‘OCB-115’, ‘OCB-120’, ‘OCB-119’, ‘OCB-067’, ‘OCB-058’, ‘OCB-086’, ‘OCB-062’, ‘OCB-117’, ‘OCB-057’, ‘OCB-126’, ‘OCB-188’, ‘OCB-129’, and/or ‘OCB-includes LG1 marker 765.

In some examples, ‘OCB-029’, ‘OCB-017’, ‘OCB-022’, ‘OCB-009’, ‘OCB-011’, ‘OCB-027’, ‘OCB-019’, ‘OCB-031’, ‘OCB-121’, ‘OCB-174’, ‘OCB-169’, ‘OCB-153’, ‘OCB-115’, ‘OCB-120’, ‘OCB-119’, ‘OCB-067’, ‘OCB-058’, ‘OCB-086’, ‘OCB-062’, ‘OCB-117’, ‘OCB-057’, ‘OCB-126’, ‘OCB-188’, ‘OCB-129’, and/or ‘OCB-051’ includes LG3. In some examples, an F1, F2, F3, F4, F5, F6, F7, F8, F9, or F10 generation of ‘OCB-029’, ‘OCB-017’, ‘OCB-022’, ‘OCB-009’, ‘OCB-011’, ‘OCB-027’, ‘OCB-019’, ‘OCB-031’, ‘OCB-121’, ‘OCB-174’, ‘OCB-169’, ‘OCB-153’, ‘OCB-115’, ‘OCB-120’, ‘OCB-119’, ‘OCB-067’, ‘OCB-058’, ‘OCB-086’, ‘OCB-062’, ‘OCB-117’, ‘OCB-057’, ‘OCB-126’, ‘OCB-188’, ‘OCB-129’, and/or ‘OCB-051’ includes LG3. In some examples, progeny of ‘OCB-029’, ‘OCB-017’, ‘OCB-022’, ‘OCB-009’, ‘OCB-011’, ‘OCB-027’, ‘OCB-019’, ‘OCB-031’, ‘OCB-121’, ‘OCB-174’, ‘OCB-169’, ‘OCB-153’, ‘OCB-115’, ‘OCB-120’, ‘OCB-119’, ‘OCB-067’, ‘OCB-058’, ‘OCB-086’, ‘OCB-062’, ‘OCB-117’, ‘OCB-057’, ‘OCB-126’, ‘OCB-188’, ‘OCB-129’, and/or ‘OCB-includes LG3.

In some examples, ‘OCB-029’, ‘OCB-017’, ‘OCB-022’, ‘OCB-009’, ‘OCB-011’, ‘OCB-027’, ‘OCB-019’, ‘OCB-031’, ‘OCB-121’, ‘OCB-174’, ‘OCB-169’, ‘OCB-153’, ‘OCB-115’, ‘OCB-120’, ‘OCB-119’, ‘OCB-067’, ‘OCB-058’, ‘OCB-086’, ‘OCB-062’, ‘OCB-117’, ‘OCB-057’, ‘OCB-126’, ‘OCB-188’, ‘OCB-129’, and/or ‘OCB-051’ includes LG26. In some examples, an F1, F2, F3, F4, F5, F6, F7, F8, F9, or F10 generation of ‘OCB-029’, ‘OCB-017’, ‘OCB-022’, ‘OCB-009’, ‘OCB-011’, ‘OCB-027’, ‘OCB-019’, ‘OCB-031’, ‘OCB-121’, ‘OCB-174’, ‘OCB-169’, ‘OCB-153’, ‘OCB-115’, ‘OCB-120’, ‘OCB-119’, ‘OCB-067’, ‘OCB-058’, ‘OCB-086’, ‘OCB-062’, ‘OCB-117’, ‘OCB-057’, ‘OCB-126’, ‘OCB-188’, ‘OCB-129’, and/or ‘OCB-051’ includes LG26. In some examples, progeny of ‘OCB-029’, ‘OCB-017’, ‘OCB-022’, ‘OCB-009’, ‘OCB-011’, ‘OCB-027’, ‘OCB-019’, ‘OCB-031’, ‘OCB-121’, ‘OCB-174’, ‘OCB-169’, ‘OCB-153’, ‘OCB-115’, ‘OCB-120’, ‘OCB-119’, ‘OCB-067’, ‘OCB-058’, ‘OCB-086’, ‘OCB-062’, ‘OCB-117’, ‘OCB-057’, ‘OCB-126’, ‘OCB-188’, ‘OCB-129’, and/or ‘OCB-includes LG26. In some examples, ‘OCB-029’, ‘OCB-017’, ‘OCB-022’, ‘OCB-009’, ‘OCB-011’, ‘OCB-027’, ‘OCB-019’, ‘OCB-031’, ‘OCB-121’, ‘OCB-174’, ‘OCB-169’, ‘OCB-153’, ‘OCB-115’, ‘OCB-120’, ‘OCB-119’, ‘OCB-067’, ‘OCB-058’, ‘OCB-086’, ‘OCB-062’, ‘OCB-117’, ‘OCB-057’, ‘OCB-126’, ‘OCB-188’, ‘OCB-129’, and/or ‘OCB-051’ includes LG26 marker 989212. In some examples, an F1, F2, F3, F4, F5, F6, F7, F8, F9, or F10 generation of ‘OCB-029’, ‘OCB-017’, ‘OCB-022’, ‘OCB-009’, ‘OCB-011’, ‘OCB-027’, ‘OCB-019’, ‘OCB-031’, ‘OCB-121’, ‘OCB-174’, ‘OCB-169’, ‘OCB-153’, ‘OCB-115’, ‘OCB-120’, ‘OCB-119’, ‘OCB-067’, ‘OCB-058’, ‘OCB-086’, ‘OCB-062’, ‘OCB-117’, ‘OCB-057’, ‘OCB-126’, ‘OCB-188’, ‘OCB-129’, and/or ‘OCB-051’ includes LG26 marker 989212. In some examples, progeny of ‘OCB-029’, ‘OCB-017’, ‘OCB-022’, ‘OCB-009’, ‘OCB-011’, ‘OCB-027’, ‘OCB-019’, ‘OCB-031’, ‘OCB-121’, ‘OCB-174’, ‘OCB-169’, ‘OCB-153’, ‘OCB-115’, ‘OCB-120’, ‘OCB-119’, ‘OCB-067’, ‘OCB-058’, ‘OCB-086’, ‘OCB-062’, ‘OCB-117’, ‘OCB-057’, ‘OCB-126’, ‘OCB-188’, ‘OCB-129’, and/or ‘OCB-includes LG26 marker 989212.

The disclosure includes a tissue culture of regenerable cells of cultivar ‘OCB-029’, ‘OCB-017’, ‘OCB-022’, ‘OCB-009’, ‘OCB-011’, ‘OCB-027’, ‘OCB-019’, ‘OCB-031’, ‘OCB-121’, ‘OCB-174’, ‘OCB-169’, ‘OCB-153’, ‘OCB-115’, ‘OCB-120’, ‘OCB-119’, ‘OCB-067’, ‘OCB-058’, ‘OCB-086’, ‘OCB-062’, ‘OCB-117’, ‘OCB-057’, ‘OCB-126’, ‘OCB-188’, ‘OCB-129’, and ‘OCB-051’, as well as parent ‘CB15’, and plants regenerated therefrom. Such regenerated plants can include, consist essentially of, or consist of the physiological and morphological characteristics of a plant grown from the seed of cultivar ‘OCB-029’, ‘OCB-017’, ‘OCB-022’, ‘OCB-009’, ‘OCB-011’, ‘OCB-027’, ‘OCB-019’, ‘OCB-031’, ‘OCB-121’, ‘OCB-174’, ‘OCB-169’, ‘OCB-153’, ‘OCB-115’, ‘OCB-120’, ‘OCB-119’, ‘OCB-067’, ‘OCB-058’, ‘OCB-086’, ‘OCB-062’, ‘OCB-117’, ‘OCB-057’, ‘OCB-126’, ‘OCB-188’, ‘OCB-129’, and ‘OCB-051’ as well as parent ‘CB15’. Exemplary regenerable cells include but are not limited to those from protoplasts or cells, such as those from leaf, stem, protoplast, pollen, ovule, embryo, cotyledon, hypocotyl, meristematic cell, root, root tip, pistil, anther, flower, petal, seed, shoot, stein, or petiole of cultivar ‘OCB-029’, ‘OCB-017’, ‘OCB-022’, ‘OCB-009’, ‘OCB-011’, ‘OCB-027’, ‘OCB-019’, ‘OCB-031’, ‘OCB-121’, ‘OCB-174’, ‘OCB-169’, ‘OCB-153’, ‘OCB-115’, ‘OCB-120’, ‘OCB-119’, ‘OCB-067’, ‘OCB-058’, ‘OCB-086’, ‘OCB-062’, ‘OCB-117’, ‘OCB-057’, ‘OCB-126’, ‘OCB-188’, ‘OCB-129’, and ‘OCB-051’, as well as parent ‘CB15’, provided herein.

The disclosure provides the detailed description for new basils that arose from a series of crosses that led to varieties having, consisting essentially of, or consisting of, the morphological and physiological characteristics of the new varieties provided herein ‘OCB-029’, ‘OCB-017’, ‘OCB-022’, ‘OCB-009’, ‘OCB-011’, ‘OCB-027’, ‘OCB-019’, ‘OCB-031’, ‘OCB-121’, ‘OCB-174’, ‘OCB-169’, ‘OCB-153’, ‘OCB-115’, ‘OCB-120’, ‘OCB-119’, ‘OCB-067’, ‘OCB-058’, ‘OCB-086’, ‘OCB-062’, ‘OCB-117’, ‘OCB-057’, ‘OCB-126’, ‘OCB-188’, ‘OCB-129’, and ‘OCB-051’), such as the characteristics noted in Table 1, for example chilling tolerance and in some examples also downy mildew resistance/tolerance (e.g., see Table 1). In some examples, the cultivars ‘OCB-029’, ‘OCB-017’, ‘OCB-022’, ‘OCB-009’, ‘OCB-011’, ‘OCB-027’, ‘OCB-019’, ‘OCB-031’, ‘OCB-121’, ‘OCB-174’, ‘OCB-169’, ‘OCB-153’, ‘OCB-115’, ‘OCB-120’, ‘OCB-119’, ‘OCB-067’, ‘OCB-058’, ‘OCB-086’, ‘OCB-062’, ‘OCB-117’, ‘OCB-057’, ‘OCB-126’, ‘OCB-188’, ‘OCB-129’, and ‘OCB-051’ provided herein have resistance/tolerance to BDM (Table 1). In other embodiments, a basil plant or part thereof (such as an essential oil) provided herein includes a chemical profile as set forth herein, for example sweet basil aroma types are characterized by high linalool content, a moderate eucalyptol content and low estragole content while Thai basil aroma types are characterized by high estragole content and moderate to high linalool content (e.g., see Table 1). However, the specific essential oil composition of such a plant may vary with time of harvest, growing area, season, etc. (Lee et al., Perfumer & Flavorist 42:37-40,42-50, 2017).

Also provided are genetically modified or genetically edited versions of ‘OCB-029’, ‘OCB-017’, ‘OCB-022’, ‘OCB-009’, ‘OCB-011’, ‘OCB-027’, ‘OCB-019’, ‘OCB-031’, ‘OCB-121’, ‘OCB-174’, ‘OCB-169’, ‘OCB-153’, ‘OCB-115’, ‘OCB-120’, ‘OCB-119’, ‘OCB-067’, ‘OCB-058’, ‘OCB-086’, ‘OCB-062’, ‘OCB-117’, ‘OCB-057’, ‘OCB-126’, ‘OCB-188’, ‘OCB-129’, and ‘OCB-051’, as well as parent ‘CB15’, such as those that include one or more transgenes that confer a desirable trait, such as those that include one or more gene edits, such as those that include one or more base edits, such as one or more of those provided herein. In one example, the ‘OCB-029’, ‘OCB-017’, ‘OCB-022’, ‘OCB-009’, ‘OCB-011’, ‘OCB-027’, ‘OCB-019’, ‘OCB-031’, ‘OCB-121’, ‘OCB-174’, ‘OCB-169’, ‘OCB-153’, ‘OCB-115’, ‘OCB-120’, ‘OCB-119’, ‘OCB-067’, ‘OCB-058’, ‘OCB-086’, ‘OCB-062’, ‘OCB-117’, ‘OCB-057’, ‘OCB-126’, ‘OCB-188’, ‘OCB-129’, or ‘OCB-051’ variety as well as parent ‘CB15’, is modified using gene editing (such as gene silencing with CRISPR/Cas system, such as CRISPR/Cas9, or base editing).

Compositions that include a seed (such as a seed of ‘‘OCB-029’, ‘OCB-017’, ‘OCB-022’, ‘OCB-009’, ‘OCB-011’, ‘OCB-027’, ‘OCB-019’, ‘OCB-031’, ‘OCB-121’, ‘OCB-174’, ‘OCB-169’, ‘OCB-153’, ‘OCB-115’, ‘OCB-120’, ‘OCB-119’, ‘OCB-067’, ‘OCB-058’, ‘OCB-086’, ‘OCB-062’, ‘OCB-117’, ‘OCB-057’, ‘OCB-126’, ‘OCB-188’, ‘OCB-129’, and/or ‘OCB-051’, as well as parent ‘CB15’) that produces a plant of the disclosure in plant seed growth media are provided. Examples of plant seed growth media include soil and synthetic cultivation medium (e.g., those that include polymers and/or hydrogels), and others known in the art (e.g., see U.S. Pat. No. 4,241,537). The growth media can be in a container or can, for example, be soil or soilless media in a field or greenhouse. Plant seed growth media can provide adequate physical support for seeds and can retain moisture and/or nutritional components. Examples of characteristics for soils can be found, for instance, in U.S. Pat. Nos. 3,932,166 and 4,707,176.

Also provided is a tissue culture of regenerable cells of a disclosed basil plant (‘OCB-029’, ‘OCB-017’, ‘OCB-022’, ‘OCB-009’, ‘OCB-011’, ‘OCB-027’, ‘OCB-019’, ‘OCB-031’, ‘OCB-121’, ‘OCB-174’, ‘OCB-169’, ‘OCB-153’, ‘OCB-115’, ‘OCB-120’, ‘OCB-119’, ‘OCB-067’, ‘OCB-058’, ‘OCB-086’, ‘OCB-062’, ‘OCB-117’, ‘OCB-057’, ‘OCB-126’, ‘OCB-188’, ‘OCB-129’, and ‘OCB-051’, as well as parent ‘CB15’), such as one that exhibits chilling tolerance as well as plants regenerated therefrom which may express some or all of the physiological and morphological characteristics of a disclosed basil plant.

Disclosed are new basil varieties (‘OCB-029’, ‘OCB-017’, ‘OCB-022’, ‘OCB-009’, ‘OCB-011’, ‘OCB-027’, ‘OCB-019’, ‘OCB-031’, ‘OCB-121’, ‘OCB-174’, ‘OCB-169’, ‘OCB-153’, ‘OCB-115’, ‘OCB-120’, ‘OCB-119’, ‘OCB-067’, ‘OCB-058’, ‘OCB-086’, ‘OCB-062’, ‘OCB-117’, ‘OCB-057’, ‘OCB-126’, ‘OCB-188’, ‘OCB-129’, and ‘OCB-051’, as well as parent ‘CB15’), which include a single locus conversion. The single locus conversion can include a transgenic gene which has been introduced by genetic transformation. In some embodiments, the single locus conversion can include a dominant or recessive allele. The locus conversion can confer any trait upon the single locus converted plant, including nutritional value, aromatic value, herbicide resistance, insect resistance, resistance to bacterial, fungal, or viral disease, male fertility or sterility, and improved nutritional quality.

A first generation (F1) hybrid seed produced by crossing a plant of the disclosure (‘OCB-029’, ‘OCB-017’, ‘OCB-022’, ‘OCB-009’, ‘OCB-011’, ‘OCB-027’, ‘OCB-019’, ‘OCB-031’, ‘OCB-121’, ‘OCB-174’, ‘OCB-169’, ‘OCB-153’, ‘OCB-115’, ‘OCB-120’, ‘OCB-119’, ‘OCB-067’, ‘OCB-058’, ‘OCB-086’, ‘OCB-062’, ‘OCB-117’, ‘OCB-057’, ‘OCB-126’, ‘OCB-188’, ‘OCB-129’, ‘OCB-051’, or parent ‘CB15’) to a second basil plant is provided. Also provided are the F1 hybrid basil plants grown from the hybrid seed produced by such crossing, and the seeds of an F1 hybrid plant. In some embodiments, the F₁ hybrid basil plant is grown from the hybrid seed produced by crossing a new basil variety provided herein (‘OCB-029’, ‘OCB-017’, ‘OCB-022’, ‘OCB-009’, ‘OCB-011’, ‘OCB-027’, ‘OCB-019’, ‘OCB-031’, ‘OCB-121’, ‘OCB-174’, ‘OCB-169’, ‘OCB-153’, ‘OCB-115’, ‘OCB-120’, ‘OCB-119’, ‘OCB-067’, ‘OCB-058’, ‘OCB-086’, ‘OCB-062’, ‘OCB-117’, ‘OCB-057’, ‘OCB-126’, ‘OCB-188’, ‘OCB-129’, and ‘OCB-051’), as well as parent ‘CB15’, to a second basil plant (such as a second sweet or Thai basil). In specific examples, provided is a seed of an F₁ hybrid plant produced with a new basil variety provided herein as one parent, the second filial generation (F₂) basil plant grown from the seed of the F₁ hybrid plant, and the seeds of the F₂ hybrid plant. Also provided are the development of inbred lines reaching six or more generations of selfing one or more of the ‘OCB-029’, ‘OCB-017’, ‘OCB-022’, ‘OCB-009’, ‘OCB-011’, ‘OCB-027’, ‘OCB-019’, ‘OCB-031’, ‘OCB-121’, ‘OCB-174’, ‘OCB-169’, ‘OCB-153’, ‘OCB-115’, ‘OCB-120’, ‘OCB-119’, ‘OCB-067’, ‘OCB-058’, ‘OCB-086’, ‘OCB-062’, ‘OCB-117’, ‘OCB-057’, ‘OCB-126’, ‘OCB-188’, ‘OCB-129’, and/or ‘OCB-051’ cultivars, or parent ‘CB15’.

Methods of producing basil seeds are provided. Such a method can include crossing a new basil variety (‘OCB-029’, ‘OCB-017’, ‘OCB-022’, ‘OCB-009’, ‘OCB-011’, ‘OCB-027’, ‘OCB-019’, ‘OCB-031’, ‘OCB-121’, ‘OCB-174’, ‘OCB-169’, ‘OCB-153’, ‘OCB-115’, ‘OCB-120’, ‘OCB-119’, ‘OCB-067’, ‘OCB-058’, ‘OCB-086’, ‘OCB-062’, ‘OCB-117’, ‘OCB-057’, ‘OCB-126’, ‘OCB-188’, ‘OCB-129’, or ‘OCB-051’, or parent ‘CB15’) to any second basil plant (such as another sweet or Thai basil plant), including itself or another plant of the disclosure. In particular embodiments, the method of crossing includes (a) planting seeds of a basil plant provided herein; (b) cultivating basil plants resulting from the seeds until said plants bear flowers; (c) allowing fertilization of the flowers of said plants; and (d) harvesting seeds produced from said plants.

In one example, methods of producing basil seeds (such as hybrid seeds) include crossing a new basil variety (‘OCB-029’, ‘OCB-017’, ‘OCB-022’, ‘OCB-009’, ‘OCB-011’, ‘OCB-027’, ‘OCB-019’, ‘OCB-031’, ‘OCB-121’, ‘OCB-174’, ‘OCB-169’, ‘OCB-153’, ‘OCB-115’, ‘OCB-120’, ‘OCB-119’, ‘OCB-067’, ‘OCB-058’, ‘OCB-086’, ‘OCB-062’, ‘OCB-117’, ‘OCB-057’, ‘OCB-126’, ‘OCB-188’, ‘OCB-129’, and ‘OCB-051’), or parent ‘CB15’, to a second basil plant (such as another sweet or Thai basil plant), which is nonisogenic to the new basil variety. In particular examples, crossing includes cultivating basil plants grown from seeds of the new variety(ies) provided herein and cultivating basil plants grown from seeds of a second, distinct basil plant until the plants bear flowers; cross-pollinating a flower on one of the two plants with the pollen of the other plant; and harvesting the seeds resulting from the cross pollination.

Methods for developing chilling tolerant basil plants (such as sweet or Thai basil plants) in a basil breeding program are provided. Such methods can include using a plant or part thereof from a new basil variety (‘OCB-029’, ‘OCB-017’, ‘OCB-022’, ‘OCB-009’, ‘OCB-011’, ‘OCB-027’, ‘OCB-019’, ‘OCB-031’, ‘OCB-121’, ‘OCB-174’, ‘OCB-169’, ‘OCB-153’, ‘OCB-115’, ‘OCB-120’, ‘OCB-119’, ‘OCB-067’, ‘OCB-058’, ‘OCB-086’, ‘OCB-062’, ‘OCB-117’, ‘OCB-057’, ‘OCB-126’, ‘OCB-188’, ‘OCB-129’, and ‘OCB-051’), or parent ‘CB15’, as a source of breeding material using plant breeding techniques, such as but not limited to recurrent selection, mass selection, bulk selection, backcrossing, pedigree breeding, single seed descent, genetic marker-assisted selection, and genetic transformation. In certain examples, the cultivar ‘OCB-029’, ‘OCB-017’, ‘OCB-022’, ‘OCB-009’, ‘OCB-011’, ‘OCB-027’, ‘OCB-019’, ‘OCB-031’, ‘OCB-121’, ‘OCB-174’, ‘OCB-169’, ‘OCB-153’, ‘OCB-115’, ‘OCB-120’, ‘OCB-119’, ‘OCB-067’, ‘OCB-058’, ‘OCB-086’, ‘OCB-062’, ‘OCB-117’, ‘OCB-057’, ‘OCB-126’, ‘OCB-188’, ‘OCB-129’, ‘OCB-051’ or parent ‘CB15’, is used as a male or female parent.

Methods of producing a basil plant derived from a plant provided herein (‘OCB-029’, ‘OCB-017’, ‘OCB-022’, ‘OCB-009’, ‘OCB-011’, ‘OCB-027’, ‘OCB-019’, ‘OCB-031’, ‘OCB-121’, ‘OCB-174’, ‘OCB-169’, ‘OCB-153’, ‘OCB-115’, ‘OCB-120’, ‘OCB-119’, ‘OCB-067’, ‘OCB-058’, ‘OCB-086’, ‘OCB-062’, ‘OCB-117’, ‘OCB-057’, ‘OCB-126’, ‘OCB-188’, ‘OCB-129’, and ‘OCB-051’, or parent ‘CB15’), such as an inbred basil plant, are provided. Such methods can include (a) preparing a progeny plant derived from a plant of a new basil variety (‘OCB-029’, ‘OCB-017’, ‘OCB-022’, ‘OCB-009’, ‘OCB-011’, ‘OCB-027’, ‘OCB-019’, ‘OCB-031’, ‘OCB-121’, ‘OCB-174’, ‘OCB-169’, ‘OCB-153’, ‘OCB-115’, ‘OCB-120’, ‘OCB-119’, ‘OCB-067’, ‘OCB-058’, ‘OCB-086’, ‘OCB-062’, ‘OCB-117’, ‘OCB-057’, ‘OCB-126’, ‘OCB-188’, ‘OCB-129’, and ‘OCB-051’), or parent ‘CB15’, by crossing the plant with a second basil plant; and (b) crossing the progeny plant with itself or a second plant to produce a progeny plant of a subsequent generation which is derived from a plant of a disclosed new basil variety. The method can further include (c) crossing the progeny plant of a subsequent generation with itself or a second plant; and (d) repeating steps (b) and (c) for, least 2 additional generations (such as at least 3, at least 5, or at least 10 additional generations) to produce an inbred basil plant derived from a plant of a new basil variety provided herein.

Methods of producing a basil plant derived from a new basil variety ‘OCB-029’, ‘OCB-017’, ‘OCB-022’, ‘OCB-009’, ‘OCB-011’, ‘OCB-027’, ‘OCB-019’, ‘OCB-031’, ‘OCB-121’, ‘OCB-174’, ‘OCB-169’, ‘OCB-153’, ‘OCB-115’, ‘OCB-120’, ‘OCB-119’, ‘OCB-067’, ‘OCB-058’, ‘OCB-086’, ‘OCB-062’, ‘OCB-117’, ‘OCB-057’, ‘OCB-126’, ‘OCB-188’, ‘OCB-129’, or ‘OCB-051’, or parent ‘CB15’, can include (a) crossing a derived basil plant with itself or another basil plant to yield additional derived progeny basil seed; (b) growing the progeny basil seed of step (a) under plant growth conditions to yield additional derived basil plants; and (c) repeating the crossing and growing steps of (a) and (b) to generate further basil plants. In specific embodiments, steps (a) and (b) may be repeated, for example 0 to 7 times (such as 0 to 4 or 1 to 5 times, such as 1, 2, 3, 4, 5, 6, or 7 times) as desired to generate further basil plants derived from a new basil variety provided herein (‘OCB-029’, ‘OCB-017’, ‘OCB-022’, ‘OCB-009’, ‘OCB-011’, ‘OCB-027’, ‘OCB-019’, ‘OCB-031’, ‘OCB-121’, ‘OCB-174’, ‘OCB-169’, ‘OCB-153’, ‘OCB-115’, ‘OCB-120’, ‘OCB-119’, ‘OCB-067’, ‘OCB-058’, ‘OCB-086’, ‘OCB-062’, ‘OCB-117’, ‘OCB-057’, ‘OCB-126’, ‘OCB-188’, ‘OCB-129’, and ‘OCB-051’), or parent ‘CB15’.

Methods of producing basil seed from a parent plant provided herein (‘CB15’) are provided. In some examples such methods include crossing a basil variety provided herein with itself or a second basil plant (such as another sweet or Thai basil plant) and harvesting a resulting basil seed. In some examples, the second basil plant has a desirable trait, which is introduced into plants in the form of seeds resulting from such a cross. For example, the second plant can be transgenic, wherein the transgene confers the desirable trait. Seeds produced by such methods, including F₁ hybrid seeds, as well as basil plants or parts thereof produced by growing such a seed, are provided. In some examples, the method of crossing includes planting seeds of the basil variety provided herein (such as a sweet or Thai basil), cultivating basil plants resulting from the seeds until the plants bear flowers, allowing fertilization of the flowers of the plants; and harvesting seeds produced from the plants. In some examples, F₁ or subsequent generations of basil plants (e.g., F₂, F₃, F₄, F₅, F₆, F₇, F₈, F₉, or F₁₀, have chilling tolerance.

Methods are provided for producing a plant of a basil variety provided herein (‘OCB-029’, ‘OCB-017’, ‘OCB-022’, ‘OCB-009’, ‘OCB-011’, ‘OCB-027’, ‘OCB-019’, ‘OCB-031’, ‘OCB-121’, ‘OCB-174’, ‘OCB-169’, ‘OCB-153’, ‘OCB-115’, ‘OCB-120’, ‘OCB-119’, ‘OCB-067’, ‘OCB-058’, ‘OCB-086’, ‘OCB-062’, ‘OCB-117’, ‘OCB-057’, ‘OCB-126’, ‘OCB-188’, ‘OCB-129’, ‘OCB-051’, or parent ‘CB15’) that has one or more added desired agronomic traits, as well as plants and seeds generated from such methods. In one example, such a method provides a basil plant having a single locus conversion of a basil variety provided herein, wherein the basil plant includes or expresses the physiological and morphological characteristics of a new basil variety provided herein (such as those shown in Table 1). Such methods can include introducing one or more transgenes that confer one or more desired traits into a plant of a new basil variety provided herein. A transgenic or non-transgenic single locus conversion can also be introduced by backcrossing. Exemplary desired traits include herbicide tolerance or resistance, resistance or tolerance to an insect, resistance or tolerance to a bacterial disease, resistance or tolerance to a viral disease, resistance or tolerance to a fungal disease, resistance or tolerance to a nematode, resistance or tolerance to a pest, male sterility, site-specific recombination; abiotic stress tolerance (such as tolerance to drought, heat, cold, low or high soil pH level, and/or salt), modified downy mildew resistance and tolerance content, or other desired qualities.

Methods of introducing a single locus conversion (such as a desired trait) into a new basil variety disclosed herein (‘OCB-029’, ‘OCB-017’, ‘OCB-022’, ‘OCB-009’, ‘OCB-011’, ‘OCB-027’, ‘OCB-019’, ‘OCB-031’, ‘OCB-121’, ‘OCB-174’, ‘OCB-169’, ‘OCB-153’, ‘OCB-115’, ‘OCB-120’, ‘OCB-119’, ‘OCB-067’, ‘OCB-058’, ‘OCB-086’, ‘OCB-062’, ‘OCB-117’, ‘OCB-057’, ‘OCB-126’, ‘OCB-188’, ‘OCB-129’, or ‘OCB-051’), or parent ‘CB15’ are provided. In some examples the methods include (a) crossing a plant of a new basil variety disclosed herein with a second plant having one or more desired traits to produce F₁ progeny plants; (b) selecting F1 progeny plants that have the desired trait to produce selected F₂ progeny plants; (c) crossing the selected progeny plants with at least a first plant of ‘OCB-029’, ‘OCB-017’, ‘OCB-022’, ‘OCB-009’, ‘OCB-011’, ‘OCB-027’, ‘OCB-019’, ‘OCB-031’, ‘OCB-121’, ‘OCB-174’, ‘OCB-169’, ‘OCB-153’, ‘OCB-115’, ‘OCB-120’, ‘OCB-119’, ‘OCB-067’, ‘OCB-058’, ‘OCB-086’, ‘OCB-062’, ‘OCB-117’, ‘OCB-057’, ‘OCB-126’, ‘OCB-188’, ‘OCB-129’, or ‘OCB-051’, or parent ‘CB15’, to produce backcross progeny plants; (d) selecting backcross progeny plants that have the desired trait and physiological and morphological characteristics of a new basil variety disclosed herein to produce selected backcross progeny plants; and (e) repeating steps (c) and (d) one or more times in succession to produce selected second or higher backcross progeny plants that include the desired trait and the physiological and morphological characteristics of one of a new basil variety disclosed herein when grown in the same environmental conditions. In some embodiments, the single locus confers a desirable trait, such herbicide tolerance or resistance, drought tolerance, heat tolerance, low or high soil pH level tolerance, salt tolerance, resistance or tolerance to an insect, resistance or tolerance to a bacterial disease, resistance or tolerance to a viral disease, resistance or tolerance to a fungal disease, resistance or tolerance to a nematode, resistance or tolerance to a pest, male sterility, site-specific recombination, abiotic stress tolerance (such as tolerance to drought, heat, low or high soil pH level, and/or salt), and in particular modified resistance/tolerance to basil downy mildew disease, such as downy mildew resistance or tolerance. In some examples, the single locus confers the ability to synthesize a protein encoded by a gene located within the single locus.

The disclosure also provides basil plants and parts thereof produced by any of the methods disclosed herein. In some embodiments, basil plants produced by the disclosed methods includes at least one, at least two, at least three, at least four, at least five, or at least 10 of the traits of a new basil variety as described herein. In some embodiments, the basil plants produced by the disclosed methods include at least one, at least two, at least three, at least four, at least five, at least 6, at least 7, or at least 10 of the traits of a new basil variety provided herein (see Table 1), such as providing chilling tolerance, for example in combination with downy mildew resistance and/or conferring a degree of high tolerance under high disease pressure of downy mildew, as described herein.

The disclosure provides basil seed deposited as ATCC Accession No. ______.

Methods are provided for producing and using an extract or an essential oil from the new basil variety provided herein. For example, such an extract or essential oil can be used in foods, flavors, fragrances and other products.

Also provided herein is packaging material containing one or more ‘OCB-029’, ‘OCB-017’, ‘OCB-022’, ‘OCB-009’, ‘OCB-011’, ‘OCB-027’, ‘OCB-019’, ‘OCB-031’, ‘OCB-121’, ‘OCB-174’, ‘OCB-169’, ‘OCB-153’, ‘OCB-115’, ‘OCB-120’, ‘OCB-119’, ‘OCB-067’, ‘OCB-058’, ‘OCB-086’, ‘OCB-062’, ‘OCB-117’, ‘OCB-057’, ‘OCB-126’, ‘OCB-188’, ‘OCB-129’, ‘OCB-051’ and/or ‘CB15’ plants or parts thereof (such as biomass, leaves, extract, and/or oil(s)). In some examples, such a package includes cells, DNA, and/or protein from a ‘OCB-029’, ‘OCB-017’, ‘OCB-022’, ‘OCB-009’, ‘OCB-011’, ‘OCB-027’, ‘OCB-019’, ‘OCB-031’, ‘OCB-121’, ‘OCB-174’, ‘OCB-169’, ‘OCB-153’, ‘OCB-115’, ‘OCB-120’, ‘OCB-119’, ‘OCB-067’, ‘OCB-058’, ‘OCB-086’, ‘OCB-062’, ‘OCB-117’, ‘OCB-057’, ‘OCB-126’, ‘OCB-188’, ‘OCB-129’, ‘OCB-051’ and/or ‘CB15’ cultivar. Exemplary packaging material includes, but is not limited to, boxes, bags, plastic containers, bottles, jars, or other containers. Such packaging material can be made from, for example, glass, paper, or plastic.

For example, the disclosure provides a package containing a plant or part thereof from one or more of ‘OCB-029’, ‘OCB-017’, ‘OCB-022’, ‘OCB-009’, ‘OCB-011’, ‘OCB-027’, ‘OCB-019’, ‘OCB-031’, ‘OCB-121’, ‘OCB-174’, ‘OCB-169’, ‘OCB-153’, ‘OCB-115’, ‘OCB-120’, ‘OCB-119’, ‘OCB-067’, ‘OCB-058’, ‘OCB-086’, ‘OCB-062’, ‘OCB-117’, ‘OCB-057’, ‘OCB-126’, ‘OCB-188’, ‘OCB-129’, ‘OCB-051’ and ‘CB15’ (such as a bag, jar, box, bottle, or other container containing leaves and/or biomass from ‘OCB-029’, ‘OCB-017’, ‘OCB-022’, ‘OCB-009’, ‘OCB-011’, ‘OCB-027’, ‘OCB-019’, ‘OCB-031’, ‘OCB-121’, ‘OCB-174’, ‘OCB-169’, ‘OCB-153’, ‘OCB-115’, ‘OCB-120’, ‘OCB-119’, ‘OCB-067’, ‘OCB-058’, ‘OCB-086’, ‘OCB-062’, ‘OCB-117’, ‘OCB-057’, ‘OCB-126’, ‘OCB-188’, ‘OCB-129’, ‘OCB-051’, and/or ‘CB15’). In some examples, the leaves and/or biomass are dried or freeze dried. In some examples, the leaves and/or biomass are frozen. In some examples, the leaves and/or biomass are fresh. The leaves and/or biomass of ‘OCB-029’, ‘OCB-017’, ‘OCB-022’, ‘OCB-009’, ‘OCB-011’, ‘OCB-027’, ‘OCB-019’, ‘OCB-031’, ‘OCB-121’, ‘OCB-174’, ‘OCB-169’, ‘OCB-153’, ‘OCB-115’, ‘OCB-120’, ‘OCB-119’, ‘OCB-067’, ‘OCB-058’, ‘OCB-086’, ‘OCB-062’, ‘OCB-117’, ‘OCB-057’, ‘OCB-126’, ‘OCB-188’, ‘OCB-129’, ‘OCB-051’ and/or ‘CB15’ are combined with leaves and/or biomass of other basil varieties, or other materials (such as other herbs, such as oregano leaves, parsley leaves, and/or other basils apart from the disclosed sweet basils). In one example, a plant or part thereof from one or more of ‘OCB-029’, ‘OCB-017’, ‘OCB-022’, ‘OCB-009’, ‘OCB-011’, ‘OCB-027’, ‘OCB-019’, ‘OCB-031’, ‘OCB-121’, ‘OCB-174’, ‘OCB-169’, ‘OCB-153’, ‘OCB-115’, ‘OCB-120’, ‘OCB-119’, ‘OCB-067’, ‘OCB-058’, ‘OCB-086’, ‘OCB-062’, ‘OCB-117’, ‘OCB-057’, ‘OCB-126’, ‘OCB-188’, ‘OCB-129’, ‘OCB-051’ and/or ‘CB15’ (such as biomass or leaves) are part of a food or fragrance product.

An essential oil extract from one or more of ‘OCB-029’, ‘OCB-017’, ‘OCB-022’, ‘OCB-009’, ‘OCB-011’, ‘OCB-027’, ‘OCB-019’, ‘OCB-031’, ‘OCB-121’, ‘OCB-174’, ‘OCB-169’, ‘OCB-153’, ‘OCB-115’, ‘OCB-120’, ‘OCB-119’, ‘OCB-067’, ‘OCB-058’, ‘OCB-086’, ‘OCB-062’, ‘OCB-117’, ‘OCB-057’, ‘OCB-126’, ‘OCB-188’, ‘OCB-129’, ‘OCB-051’ and ‘CB15’ is also provided. In one example, the oil extract includes an aromatic oil characterized as sweet basil aroma (e.g., high linalool content, a moderate eucalyptol content and low estragole content) or Thai basil aroma types (e.g., high estragole content and moderate to high linalool content). In some examples, such an extract includes DNA and/or protein from a ‘OCB-029’, ‘OCB-017’, ‘OCB-022’, ‘OCB-009’, ‘OCB-011’, ‘OCB-027’, ‘OCB-019’, ‘OCB-031’, ‘OCB-121’, ‘OCB-174’, ‘OCB-169’, ‘OCB-153’, ‘OCB-115’, ‘OCB-120’, ‘OCB-119’, ‘OCB-067’, ‘OCB-058’, ‘OCB-086’, ‘OCB-062’, ‘OCB-117’, ‘OCB-057’, ‘OCB-126’, ‘OCB-188’, ‘OCB-129’, ‘OCB-051’ and/or ‘CB15’ cultivar.

The foregoing and other objects and features of the disclosure will become more apparent from the following detailed description, which proceeds with reference to the accompanying figures.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1. Percent necrosis of the mapping population parents in response to chilling temperatures. The parents represented the extreme phenotypic classes across all replicates with ‘CB15’ representing the chilling tolerant phenotype and ‘RU Obsession DMR’ representing the chilling sensitive phenotype. The differential response was not as extreme in the December replicate as the other replicates, yet the parents still represent the extreme phenotypes as the chilling response was lower for all individuals in this replicate. A higher percent necrosis score (100%) represented by percent area indicates chilling sensitivity and the lower percent necrosis (0%) indicates chilling tolerance. The diamond represents the mean, the central lines represent the median.

FIG. 2. Boxplot demonstrating the distribution of the F2 population's chilling response in each experiment. The October assay induced the strongest chilling response with a higher average percent necrotic score (21.3%) followed by the February assay (18.2%). The F2 responded the least to the December assay with a lower average percent necrotic score (6.8%). A higher percent necrosis score (100%) indicates chilling sensitivity and the lower percent necrosis (0%) indicates chilling tolerance. The diamond represents the mean, the central lines represent the median and the points represent outliers.

FIG. 3. Categorization of the F2 mapping population's response to chilling temperatures. Chilling tolerant F2 plants behaved similarly to the ‘CB15’ parent by exhibiting <5% necrosis while chilling sensitive F2 plants behaved similarly to the ‘RU Obsession DMR’ parent by exhibiting >30% necrosis after all three chilling assays. The F2 plants with a median response behaved similarly to the F1 hybrid by exhibiting 5-30% necrosis. Most F2 individuals displayed a median response (69%), 19% displayed a chilling tolerant response and 12% exhibited a chilling sensitive response.

FIGS. 4A-4C. Segregation of chilling response in the mapping population of ‘RU Obsession DMR’ crossed with ‘CB15’. The distribution of F2 individuals organized by median percent necrosis are displayed from the chilling evaluations in (A) October in teal, (B) December in yellow and (C) February in orange. ‘CB15’ represents the extreme chilling tolerant phenotype (left, dark turquoise) while ‘RU Obsession DMR’ represents the extreme chilling sensitive phenotype (right, light turquoise). The F1 hybrid exhibits a median response phenotype in the October and December assessment, but a median response in the February assessment (light gray). Individuals with a missing replicate lay outside the organized median (right).

FIGS. 5A-5B. Aroma compound levels of the basil mapping population parents (A) and F2 individuals (B). The parents represented the extreme phenotypic classes across all replicates with ‘CB15’ representing the high estragole, low eucalyptol and low linalool type and ‘RU Obsession DMR’ representing the low estragole, high eucalyptol and high linalool type. Among the F2 population, estragole emerged with the highest mean peak area, followed by linalool and eucalyptol. A higher peak area (A/g) indicates higher concentrations of the aroma compound per gram of plant material and the lower peak area indicates lower concentrations. The diamond represents the mean, the central lines represent the median.

FIGS. 6A-6C. Segregation of aroma compounds in the mapping population of ‘RU Obsession DMR’ crossed with ‘CB15’. The distribution of F2 individuals organized by median peak area of each compound are displayed for (A) estragole in purple, (B) eucalyptol in green and (C) linalool in pink. ‘CB15’ represents the high estragole phenotype, the low eucalyptol phenotype and the low linalool type in dark turquoise, while ‘RU Obsession DMR’ represents the low estragole phenotype, the high eucalyptol phenotype and the high linalool phenotype in light turquoise in this population specifically but not necessarily when compared to other basils. The F1 hybrid exhibits a high estragole phenotype, a median eucalyptol and a high linalool phenotype.

FIG. 7. Correlations between phenotypes. Bivariate scatter plots are shown in the bottom lefthand corner, histograms on the diagonal, and the Spearman correlation in the upper righthand corner. Chilling assays were moderately correlated with each other and aroma compounds were moderately to highly correlated with each other. There was no correlation between the results of the chilling assays and the aroma analyses.

FIG. 8. Genetic map of ‘RU Obsession DMR’ x ‘CB15’ F2 mapping population. This genetic map contains 1,761 polymorphic SNP markers on 25 linkage groups. LG21 is absent in this analysis due to insufficient quality markers. The average LG length was 90 cM (5-162 cM). The average number of markers per LG was 70.4 (13-225 SNPs).

FIG. 9. Heatmap of marker-pairwise estimated recombination fractions versus LOD scores. Markers incorporated into the genetic map are only linked with other markers assigned to the same linkage group. Marker linkage is represented by yellow shading while a lack of linkage is represented by purple shading.

FIG. 10. Significant QTL identified on LG24 in all three chilling replicates using Haley-Knott regression. The QTL on LG24 was significant across all three replicates, while the QTL on LG7 was significant in the October (teal) and December (yellow) replicates, LG13 was significant in the October replicate and LG23 was significant in the February (orange) replicate only. The LOD thresholds (α=0.05) were 3.70, 3.96 and 3.81 for the chilling assay conducted in October, December and February, respectively.

FIG. 11. Genetic map with confidence intervals for significant QTL detected in the single-QTL model in each chilling assay. A large effect QTL was located on LG24 in all three chilling assays spanning from 0.3-78 cM. Other significant QTL included those on LG7, LG13, LG16 and LG24, but were not consistent across all three experiments. Confidence intervals were determined with 95% Bayesian credible intervals. The intervals depicted were not expanded to the nearest markers.

FIG. 12. Effect plots of marker 938232 in each chilling assay. Marker 938232 was the closest marker to the QTL peak on LG24 suggesting non-additive gene action where the chilling sensitive genotype (BB) is dominant. The AA genotype represents F2 individuals that inherited two alleles from the ‘CB15’, the BB genotype represents F2 individuals that inherited two alleles from the ‘RU Obsession DMR’ parent and the AB represents F2 individuals that inherited one allele from each.

FIG. 13. Significant QTL identified for estragole and eucalyptol using Haley-Knott regression. Significant QTL were detected on LG1 and LG3 for estragole (top) and on LG26 for eucalyptol (middle). No significant QTL was detected for linalool (bottom). The LOD thresholds (α=0.05) were 3.80, 3.66 and 3.78 for estragole, eucalyptol and linalool, respectively.

FIG. 14. Genetic map with confidence intervals for significant QTL detected in the single-QTL for each aroma compound. Two QTL were detected for estragole (purple) spanning from 18.3-19.2 cM on LG1 and 65.2-70.3 cM on LG3. A QTL was detected for eucalyptol (green) spanning from 113.0-132.4 cM on LG26. Confidence intervals were determined with 95% Bayesian credible intervals. The intervals depicted were not expanded to the nearest markers.

FIG. 15. Effect plots of markers on QTL for estragole and eucalyptol.

Marker 765 was the closest marker to the QTL peak on LG1 for estragole. Marker 765 indicates non-additive gene action where the low estragole genotype (BB) is dominant. Marker 989212 was the closest marker to the QTL peak on LG26 for eucalyptol. Marker 989212 is less supportive. The AA genotype represents F2 individuals that inherited two alleles from the ‘CB15’, the BB genotype represents F2 individuals that inherited two alleles from the ‘RU Obsession DMR’ parent and the AB represents F2 individuals that inherited one allele from each.

FIG. 16. Aroma profiles of F2 selections with chilling tolerance for sweet basil cultivar development. Bar graph showing quantification of estragole, linalool and eucalyptol. Percent necrosis is also shown (diamonds) for each variety. A low percent necrosis (0-5%) represents chilling tolerance and a high percent necrosis (>30%) represents chilling sensitivity. The F2 progenitor, ‘Rutgers Obsession DMR’ is included for comparison.

FIG. 17. Aroma profiles of F2 selections with chilling tolerance for Thai basil cultivar development. Bar graph showing quantification of estragole, linalool and eucalyptol. Percent necrosis is also shown (diamonds) for each variety. A low percent necrosis (0-5%) represents chilling tolerance and a high percent necrosis (>30%) represents chilling sensitivity.

### DETAILED DESCRIPTION

The following explanations of terms and methods are provided to better describe the present disclosure and to guide those of ordinary skill in the art in the practice of the present disclosure. As used herein, “comprising” means “including” and the singular forms “a” or “an” or “the” include plural references unless the context clearly dictates otherwise. For example, reference to “comprising a plant” includes one or a plurality of such plants. The term “or” refers to a single element of stated alternative elements or a combination of two or more elements, unless the context clearly indicates otherwise. For example, the phrase “A or B” refers to A, B, or a combination of both A and B. Furthermore, the various elements, features and steps discussed herein, as well as other known equivalents for each such element, feature or step, can be mixed and matched by one of ordinary skill in this art to perform methods in accordance with principles described herein. Among the various elements, features, and steps some will be specifically included and others specifically excluded in particular examples.

Unless explained otherwise, all technical and scientific terms used herein have the same meaning as commonly understood to one of ordinary skill in the art to which this disclosure belongs. Although methods and materials similar or equivalent to those described herein can be used in the practice or testing of the present disclosure, suitable methods and materials are described below. The materials, methods, and examples are illustrative only and not intended to be limiting.

In some examples, the numbers expressing quantities of ingredients, properties such as molecular weight, reaction conditions, and so forth, used to describe and claim certain embodiments are to be understood as being modified in some instances by the term “about” or “approximately.” For example, “about” or “approximately” can indicate +/−20% variation of the value it describes. Accordingly, in some embodiments, the numerical parameters set forth herein are approximations that can vary depending upon the desired properties sought to be obtained by a particular embodiment. Notwithstanding that the numerical ranges and parameters setting forth the broad scope of some examples are approximations, the numerical values set forth in the specific examples are reported as precisely as practicable. The recitation of ranges of values herein is merely intended to serve as a shorthand method of referring individually to each separate value falling within the range.

Backcross: The mating of a plant to one of its parents. Hybrid progeny, for example, or a first generation hybrid (F1), can be crossed back one or more times to one of its parents. Backcrossing can be used to introduce one or more single locus conversions (such as one or more desirable traits) from one genetic background into another.

Basil downy mildew (BDM): The pathogen Peronospora belbahrii responsible for causing downy mildew in basil plants. Initial symptoms usually include yellow areas visible on the upper leaf surface often confined within the veins of the leaf. In situations of high disease pressure brought about by very increased levels of pathogen spores in the crop canopy brown to black leaf lesions resulting from cell death appear and may expand. Plant stunting, defoliation or death may occur if damage is severe enough.

The spectrum of resistance or tolerance through susceptibility to BDM can be measured by sporulation as opposed to leaf yellowing, which may be the result of extenuating factors such as nitrogen deficiency. Thus, response can be measured on the basis of % sporulation. In one example a scale from 0 to 4 is used, comparing all genotypes in a given test (0=no sporulation, 1=1% to 10% sporulation, 2=11% to 25% sporulation, 3=26% to 50% sporulation, and 4=51% to 100% sporulation). This scale facilitates rapid scoring of multiple leaves from individual plants or plots of multiple plants (i.e. homogenous varieties or breeding lines), while providing a repeatable and representative measure of disease reaction on an individual plant basis. For selection of individual plants, six mature leaves are detached from each plant and assigned a score from which a DSI was calculated on a single-plant basis using the equation:

$$DSI = \frac{\sum ( single leaf \times disease rating )}{( number of leaves scored \times maximum disease rating )}$$

For isogenic varieties or breeding lines replicated plots, typically between 10-15 feet, are assigned a score based on the aggregate level of sporulation among plants. In this case disease severity can be reported as the mean score across replicated plots on a given date or scoring dates can be combined and used to calculate area under the disease progress curve (AUDPC).

As used herein, “resistance” to BDM is used to describe plants for which sporulation over the growing season or cycle of the plant has not been observed in any environment and across all levels of disease pressure including the most severe.

As used herein, “tolerance” to BDM is used to describe plants exhibiting some degree of symptoms and/or sporulation that does not preclude its sale in the marketplace. Thus, total yield may decrease but not the extent of significant economic impact with regard to the sale of the product. BDM tolerance can vary according to the number and nature of genes conferring the host response. BDM tolerance can also be subject to interaction with the environment, which directly affects the level of disease pressure and will vary under different environmental conditions, inoculum density, plant age and length of season. Nevertheless, several of the disclosed new basil varieties have exhibited under greenhouse conditions no-to-few BDM symptoms (represented by low BDM scores), depending upon when the rating occurs, and are referred to herein a BDM resistant/tolerant (see Table 1).

The disclosed new basil varieties (‘OCB-029’, ‘OCB-017’, ‘OCB-022’, ‘OCB-009’, ‘OCB-011’, ‘OCB-027’, ‘OCB-019’, ‘OCB-031’, ‘OCB-121’, ‘OCB-174’, ‘OCB-169’, ‘OCB-153’, ‘OCB-115’, ‘OCB-120’, ‘OCB-119’, ‘OCB-067’, ‘OCB-058’, ‘OCB-086’, ‘OCB-062’, ‘OCB-117’, ‘OCB-057’, ‘OCB-126’, ‘OCB-188’, ‘OCB-129’, and ‘OCB-051’) may demonstrate minor differences in response by environments due to absence or presence of minor QTL. Across environments several of these varieties have comparable BDM tolerance to their progenitor ‘Rutgers Obsession DMR’.

Biomass: Organic matter derived from an organism, such as a basil plant or part thereof. In some examples, biomass refers to all the above ground plant material at a particular point of time, thus including the leaves, stems and may include flowers (at varying stages of development given the flowering period ranges over a period of time). Biomass can include all vegetative and reproductive material produced by the plant at time of harvest.

Cell. Cell as used herein includes a plant cell, whether isolated, in tissue culture or incorporated in a plant or plant part.

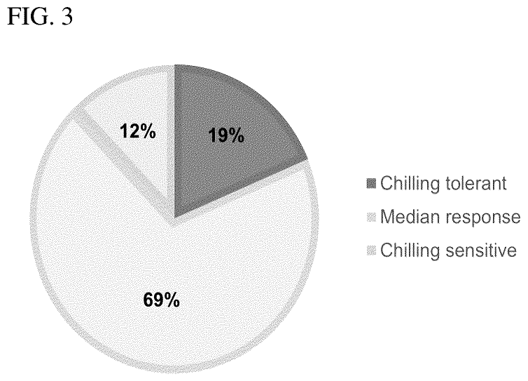

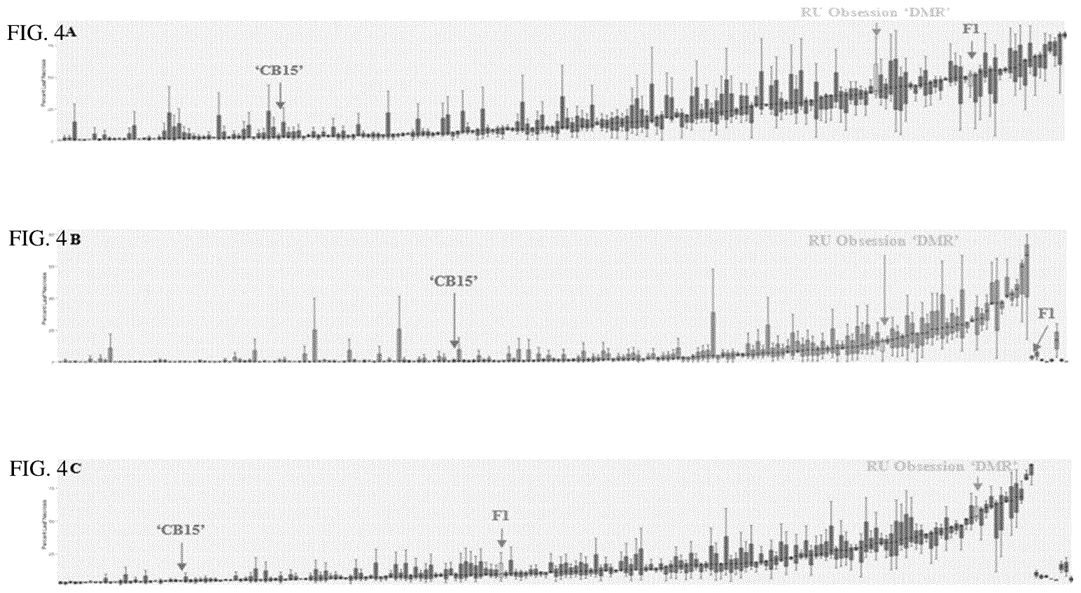

Chilling/Cold tolerance: The ability of a plant to survive and grow (e.g., withstand) in an environment of less than about 15° C. such as 0 to 15° C., 2 to 15° C., 4 to 10° C., 3 to 5° C., or 4 to 5° C., for an extended period, such as at least 1 day, at least 2 days, at least 3 days, at least 4 days, or at least 5 days (such as 2-10 days, 2-5 days, 3-5 days, or 4-5 days). In one example, chilling tolerance is <5% necrosis in the cut leaves after 4 days at 3-5° C. In one example, chilling sensitivity is >30% necrosis in the same conditions (FIG. 3). Thus, plants with chilling or cold tolerance display fewer or no chilling injuries, such as chlorosis, necrosis, wilting tissue or organ collapse (e.g., leaves, stems) or growth retardation, as compared to that observed with a plant of the same type, genus or species. Chilling tolerance can include plants (e.g., basil plants) that are intact plants with or without roots, and/or detached stems and leaves, or leaves only, and refers to the plants or parts of plants observable response after being exposed or subjected to chilling/cold temperatures. This could occur when growing in the field or greenhouse and/or in pots and containers (following episodes of low temperatures that can induce chilling injury), or at any time post-harvest during handling/processing, storage, shipping/transport/distribution and/or at the retail or consumer end.

In some examples, chilling tolerance is evidenced by a low percent leaf necrosis (e.g., <5%), for example when evaluated by the machine learning program, Leaf Necrosis Classifier (LNC). In some examples, chilling tolerance is <5% necrosis in cut leaves after 4 days at 3-5° C. In some examples, chilling sensitivity is >30% necrosis in cut leaves after 4 days at 3-5° C.

Leaves can be evaluated when attached to the plant, for example after exposing the plant to temperatures of about 3-5° C. for an extended period (for example by growing the plant outdoors), such as at least 1 day, at least 2 days, at least 3 days, at least 4 days, or at least 5 days (such as 2-10 days, 2-5 days, 3-5 days, or 4-5 days). Leaves can be evaluated following their removal from the plant and exposure to temperatures of about 3-5° C. for an extended period, such as at least 1 day, at least 2 days, at least 3 days, at least 4 days, or at least 5 days (such as 2-10 days, 2-5 days, 3-5 days, or 4-5 days).

All of the disclosed new basil varieties (‘OCB-029’, ‘OCB-017’, ‘OCB-022’, ‘OCB-009’, ‘OCB-011’, ‘OCB-027’, ‘OCB-019’, ‘OCB-031’, ‘OCB-121’, ‘OCB-174’, ‘OCB-169’, ‘OCB-153’, ‘OCB-115’, ‘OCB-120’, ‘OCB-119’, ‘OCB-067’, ‘OCB-058’, ‘OCB-086’, ‘OCB-062’, ‘OCB-117’, ‘OCB-057’, ‘OCB-126’, ‘OCB-188’, ‘OCB-129’, and ‘OCB-051’) are chilling tolerant in that exposure of leaves removed from the plant to temperatures of about 3-5° C. for an extended period, such as at least 1 day, at least 2 days, at least 3 days, at least 4 days, or at least 5 days (such as 2-10 days, 2-5 days, 3-5 days, or 4-5 days) have a low average percent leaf necrosis (e.g., <5%).

Clustered regularly interspaced short palindromic repeats (CRISPR)-CRISPR associated (Cas) systems: CRISPR-Cas is an adaptive immune system existing in most bacteria and archaea, preventing them from being infected by phages, viruses and other foreign genetic elements. It comprises CRISPR repeat-spacer arrays, which upon transcription generates CRISPR RNA (crRNA) and optionally trans-activating CRISPR RNA (tracrRNA), and a set of Cas genes which encode Cas proteins with endonuclease activity. CRISPR-Cas systems can be classified into 2 classes (Class 1 and Class 2), 6 types (I to VI) and several subtypes, with multi-Cas protein effector complexes in Class 1 systems (Type I, III, and IV) and a single effector protein in Class 2 systems (Type II, V, and VI). CRISPR/Cas systems can be used for nucleic acid (DNA and RNA) targeting or editing, for example to detect a target nucleic acid, or cut or modify a target nucleic acid at any desired location (including adding, disrupting or changing the sequence of specific genes), for example in a basil cell or plant provided herein.

The CRISPR repeat-spacer array (or CRISPR array) is a defining feature of CRISPR-Cas systems. The term “CRISPR” refers to the architecture of the array which includes constant direct repeats (DRs) interspaced with the variable spacers. In some examples, a CRISPR array includes at least a DR-spacer-DR-spacer.

Cas proteins provide the enzymatic machinery required for acquiring new spacers targeting invading elements and cleaving these elements upon subsequent encountering. Numerous Cas proteins such as Cas9, Cas12 (Cpf1), and Cas13 have been exploited to develop new tools for genome engineering.

Cas9 cleaves DNA and possesses two nuclease domain (HNH and RuvC), each cleaving one strand of the target double-stranded DNA. Catalytically inactive (deactivated) Cas9 (dCas9) is also encompassed by this disclosure. In some examples, a dCas9 includes one or more of the following point mutations: D10A, 5 H840A, and N863A. Cas proteins Cas9 nucleic acid and protein sequences are publicly available. For example, GenBank® Accession Nos. nucleotides 796693..800799 of CP012045.1 and nucleotides 1100046..1104152 of CP014139.1 disclose Cas9 nucleic acids, and GenBank® Accession Nos. AMA70685.1 and AKP81606.1 disclose Cas9 proteins. In some examples, the Cas9 is a deactivated form of Cas9 (dCas9), such as one that is nuclease deficient (e.g., those shown in GenBank® Accession Nos. AKA60242.1 and KR011748.1). In certain examples, Cas9 has at least 80% sequence identity, for example at least 85%, at least 90%, at least 95%, at least 96%, at least 97%, at least 98%, or at least 99% sequence identity to such sequences, and retains the ability to cut DNA.

CRISPR RNA (crRNA): The RNA strand responsible for hybridizing with target DNA sequences, and recruiting CRISPR endonucleases and/or CRISPR-associated effectors. crRNAs may be naturally occurring, or may be artificially synthesized.

Cross. Synonymous with hybridize or crossbreed. Includes the mating of genetically different individual plants, such as the mating of two parent plants.

Cross-pollination: Fertilization by the union of two gametes from different plants.

Essential oil (EO): A concentrated hydrophobic liquid containing aromatic volatile aroma compounds from aromatic plants, such as a basil plant. An oil is “essential” in the sense that it historically was considered by some to be the “essence of” the plant's fragrance; it does not mean indispensable. The essential oil of basil accumulates in glandular trichomes in leaves and flowers of the basil plant and these compounds impart the characteristic aroma/odor of aromatic plants/culinary herbs including basil. Methods of generating or obtaining an EO from a plant include extraction by distillation (e.g., by using steam or water or combination), expression, solvent extraction, absolute oil extraction, super critical fluid extraction, cold pressing, or combinations thereof. Methods may also include capturing such aromas that are naturally volatizing from the plant using static or nonstatic headspace above the plant material in an enclosed vial or chamber from which the volatiles are then captured.

F1 hybrid: The first generation progeny of the cross of two stable parents that are nonisogenic or isogenic plants.

Gene Silencing. A general term describing epigenetic processes of gene regulation, including any technique or mechanism in which the expression of a gene is prevented.

Genotype. The genetic constitution of a cell, an organism, or an individual (i.e., the specific allele makeup of the individual) usually with reference to a specific character under consideration.

Guide sequence/nucleic acid: A polynucleotide sequence having sufficient complementarity with a target polynucleotide sequence to hybridize with the target sequence and direct sequence-specific binding of a nuclease (such as a DNA endonuclease, such as a Cas protein) to the target sequence, and/or any polynucleotide sequence providing for such a polynucleotide sequence. Guide sequence/nucleic acid as used herein can refer to the final product (e.g., guide RNA or gRNA) that binds with a nuclease and hybridizes with a target sequence, optionally any nucleic acid intermediate/precursor that can be processed into the final product, and/or the DNA sequence from which the final product or the intermediate/precursor is transcribed. A guide sequence/nucleic acid may be a single nucleic acid molecule or comprise multiple nucleic acid molecules. In one embodiment, a guide nucleic acid comprises two parts, one being or encoding for a CRISPR RNA (crRNA), a sequence complementary to the target DNA, the other being or encoding for a trans-activating CRISPR RNA (tracrRNA), serving as a binding scaffold for a Cas endonuclease (e.g., Cas9). In another embodiment, the guide nucleic acid is a single RNA molecule, e.g., single guide RNA (sgRNA), a synthetic crRNA/tracrRNA hybrid. In yet another embodiment, the guide nucleic acid is a single crRNA that functions together with a Cpf1 endonuclease.

A guide nucleic acid can include modified bases or chemical modifications (e.g., see Latorre et al., Angewandte Chemie 55:3548-50, 2016).

In some embodiments, the degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g., the Burrows Wheeler Aligner), ClustalW, Clustal X, BLAT, Novoalign (Novocraft Technologies, ELAND (Illumina, San Diego, Calif.), SOAP (available at soap.genomics.org.cn), and Maq (available at maq.sourceforge.net). In some embodiments, a guide sequence is about, or at least about, 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some embodiments, a guide sequence is less than about 75, 50, 45, 40, 35, 30, 25, 20, 15, 12, or fewer nucleotides in length. In some embodiments, a guide sequence is 15-25 nucleotides (such as 18-22 or 18 nucleotides).

The ability of a guide sequence to direct sequence-specific binding of a CRISPR complex to a target sequence may be assessed by a suitable assay. For example, the components of a CRISPR system sufficient to form a CRISPR complex, including the guide sequence to be tested, may be provided to a host cell having the corresponding target sequence, such as by transfection with vectors encoding the components of the CRISPR sequence, followed by an assessment of preferential cleavage within the target sequence, such as by Surveyor assay. Similarly, cleavage of a target polynucleotide sequence may be evaluated in a test tube by providing the target sequence, components of a CRISPR complex, including the guide sequence to be tested and a control guide sequence different from the test guide sequence, and comparing binding or rate of cleavage at the target sequence between the test and control guide sequence reactions.

Plant: Includes reference to an immature or mature whole plant, including a plant from which seed, roots or leaves have been removed. Seed or embryo that can produce the plant is also considered to be the plant.

Plant parts. Includes protoplasts, leaves, stems, roots, root tips, anthers, pistils, seed, embryo, pollen, ovules, cotyledon, hypocotyl, flower, shoot, tissue, petiole, cells, meristematic cells and the like. Includes plant cells of a tissue culture from which basil plants can be regenerated.

Progeny. Offspring; descendants.

Regeneration. The development of a plant from tissue culture. The cells may, or may, not have been genetically modified. Plant tissue culture relies on the fact that all plant cells have the ability to generate a whole plant (totipotency). Single cells (protoplasts), pieces of leaves, or roots can often be used to generate a new plant on culture media given the required nutrients and plant hormones.

Self-pollination: The transfer of pollen from the anther to the stigma of the same plant.

Single locus converted (conversion) plant: Plants developed by backcrossing and/or by genetic transformation, wherein essentially all of the desired morphological and physiological characteristics of a basil variety are recovered in addition to the characteristics of the single locus transferred into the variety via the backcrossing technique and/or by genetic transformation.

Sweet basil: As used herein, sweet basil or sometimes referred to as American Basil, French Basil, Italian Basil refers to a plant from the genus and species Ocimum basilicum having a distinct floral aroma that can be floral, sweet, fresh, clove-like and/or spicy. They typically have medium to large leaves that cup downward. The leaves and stems are typically green in color while the flowers tend to be white. As used herein, sweet basil may also refer to a variant, progeny, or offspring of such a plant, including a plant or part thereof. The terms variety, cultivar, or the like may be used interchangeably to refer to a plant of the present disclosure.

Thai basil: A type of Ocimum basilicum of the variety thyrsiflora. Its aroma is often described as anise- and licorice-like and slightly spicy. Thai basil typically has small, narrow leaves, purple stems, and pink-purple flowers.

Tissue culture: A composition that includes isolated cells of the same or a different type or a collection of such cells organized into parts of a plant.

Transformation. The introduction of new genetic material (e.g., exogenous transgenes) into plant cells. Exemplary mechanisms that can be used to transfer nucleic acid molecules into plant cells include (but not limited to) electroporation, microprojectile bombardment, Agrobacterium-mediated transformation and direct DNA uptake by protoplasts or other plant cells.

Transgene. A gene or genetic material that has been transferred into the genome of a plant, for example by genetic engineering methods. Exemplary transgenes include cDNA (complementary DNA) segment, which is a copy of mRNA (messenger RNA), and the gene itself residing in its original region of genomic DNA. In one example, describes a segment of DNA containing a gene sequence that is introduced into the genome of a basil plant or plant cell. This non-native segment of DNA may retain the ability to produce RNA or protein in the transgenic plant, or it may alter the normal function of the transgenic plant's genetic code. In general, the transferred nucleic acid is incorporated into the plant's germ line. Transgene can also describe any DNA sequence, regardless of whether it contains a gene coding sequence or it has been artificially constructed, which has been introduced into a plant or vector construct in which it was previously not found.

### New Chilling Tolerant Basil Cultivars

Sweet basil, Ocimum basilicum L. is a popular culinary herb grown worldwide. To extend shelf life and reduce postharvest loss, most herbs and vegetables are stored and shipped at low temperatures to reduce transpiration, inhibit senescence, postharvest pathogens and to maintain freshness and texture. However, basil is a chilling sensitive species with injury appearing when fresh leaves are subjected to temperatures as low as 12° C., and severe injuries occurring as low as 10° C. [38-40]. This limits the inclusion of shipping sweet basil with other fresh produce leading growers and supply chains to use costly separate shipping arrangements or risk significant losses. Currently, there are no traditional sweet basils commercially available with significant chilling tolerance.

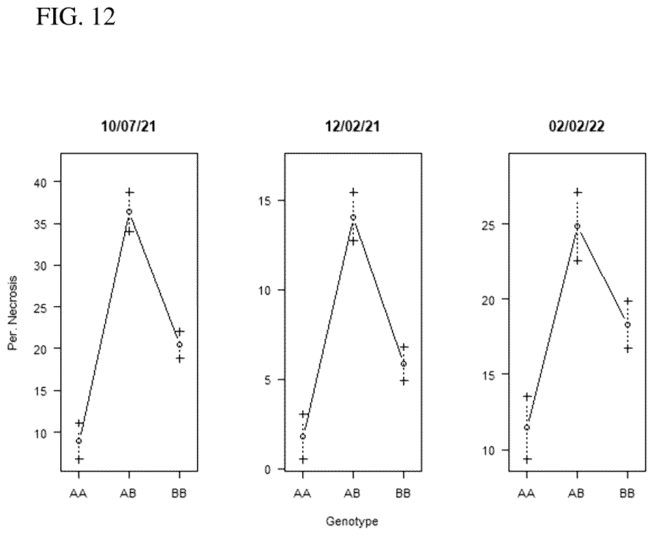

The basil line ‘CB15’ has chilling tolerance, though the aroma profile does not match a traditional sweet basil (instead it is high in estragole and has a licorice aroma). This line was crossed with O. basilicum cv. ‘Rutgers Obsession DMR’ (U.S. Pat. No. 10,159,212), a commercial sweet basil with chilling sensitivity and desirable traits, including a traditional aroma profile (high in linalool and eucalyptol and has a floral, spicy and sweet basil aroma), downy mildew tolerance, Fusarium oxysporum f. sp. basilica (FOB) resistance/tolerance, late flowering and moderate leaf size. Chilling tolerance was observed to be a heritable trait in the F2 progeny (FIGS. 1-3). Chilling tolerance was set as <5% necrosis in the cut leaves after 4 days at 3-5° C., while chilling sensitivity was set as >30% necrosis in the same conditions (FIG. 3). These delineations were selected to reflect the response of the chilling tolerant and chilling susceptible parents. As a result, only 19% of the F2 population had a chilling tolerant response while 88% had a median or highly chilling sensitive response. Thus, the gene action is likely non-additive or dominant for chilling sensitivity, which is also supported by the marker effects within qCH24 (FIG. 12).

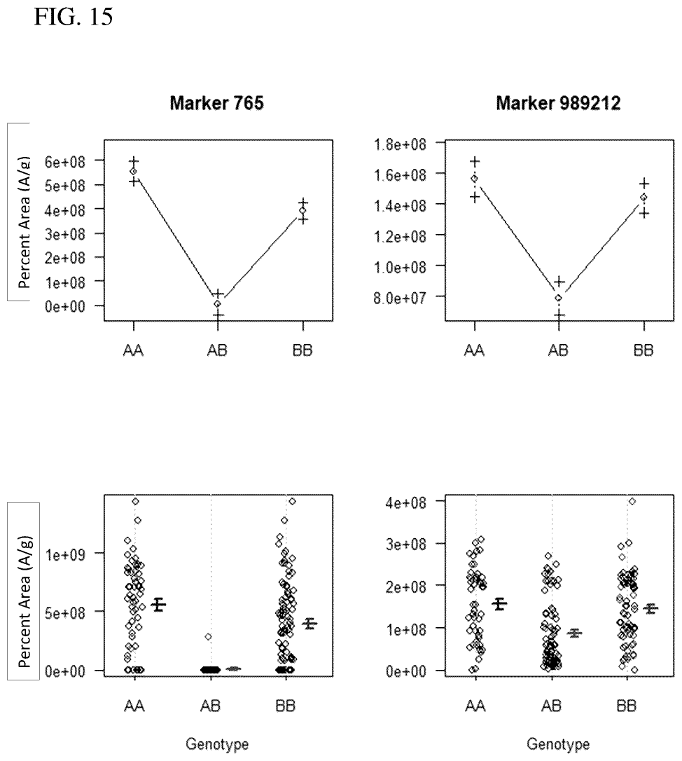

The aromatic profile of ‘CB15’ was high in estragole with a licorice aroma, while RU Obsession DMR’ is high in linalool and eucalyptol and has a floral sweet basil aroma with cooling spicy notes. No estragole or low levels of estragole were detected in a majority of F2 individuals, indicating that the gene action is likely non-additive or dominant for the low estragole phenotype. These results are supported by the marker effect plots (FIG. 15).

The final genetic map yielded 1,761 SNPs across 25 linkage groups, averaging 90 cM distance between markers over a total map distance of 2,241.8 cM. These results are comparable to the de novo genetic linkage map of an O. basilicum biparental F2 population that also used the parent ‘SB22’ [16]. The final number of mappable SNPs is significantly lower than the other known genetic linkage map for O. basilicum generated from aligning the reads of a ‘Perrie’ and ‘Cardinal’ F2 mapping population to a contiguous draft reference genome [19]. The difference in SNP yield may be due to relatively high homozygosity or recent admixture between the parents [77], stricter filtering parameters and the use of a chromosome-level reduced genome, which reduces the risk of false positives [78].

The resulting mapping population segregated in response to low temperature and aroma profile. Single nucleotide polymorphism (SNP) markers were generated from genomic sequences derived from double digestion restriction-site associated DNA sequencing (ddRADseq) and converted to genotyping data using a reference genome alignment. A genetic linkage map was constructed and seven significant QTL were identified in response to chilling temperatures with one QTL on linkage group (LG) 24 consistent across all three replicates. No QTL was identified for linalool, as the population did not segregate enough to detect this trait. Two significant QTL were identified for estragole with one QTL on LG1 being significant following multiple-QTL model (MQM). One significant QTL was identified for eucalyptol on LG26.

Genetic linkage mapping on the F2 progeny identified genomic regions associated with chilling tolerance and aroma compounds. One significant genomic region was identified on LG 24 as being related to cold tolerance. Two genomic regions were identified on LG 1 and LG3 in relationship to estragole and one significant genomic region for eucalyptol on LG26.

Potential gene candidates were identified through genome annotation on the QTLs associated with chilling. Three putative genes on LG24, which contains the major QTL for chilling response, were annotated as proteins that could be associated with cold response, including universal stress protein 13, omega-3 fatty acid desaturase (FAD), 14-3-3 protein 9 and an abscisic acid (ABA) responsive element binding factor (Table 3). Desaturation of fatty acids via omega-3 FAD and other FADs have been shown to improve cold tolerance through transcriptomic, lipidomics and transgenic studies in Tripsacum, rice and other plants [82-84]. Similarly, overexpression of 14-3-3 proteins improves cold tolerance and in Arabidopsis [74]. Cold activation of CRPK1 phosphorylates 14-3-3 proteins and leads to the degradation of CBF1 and CBF3 [73]. This negatively regulates COR expression and freezing tolerance, though no CBF or COR genes were annotated on the QTLs associated with chilling. Conversely, applications of ABA have shown to improve plant response to cold stress [70, 71]. ABA mediates abscisic acid responsive element (ABRE) binding factors (ABFs), which can be cold inducible [85]. Putative genes associated with cold temperature and stress were also identified on the linkage groups that contained minor QTLs associated with chilling response, i.e., LG7, LG13, LG16 and L23. These included cold shock, general stress, LEA, FADs, 14-3-3, ABA related and proline related proteins. Late embryogenesis abundant (LEA) and proline related proteins are of interest for their highly conservative roles under cold stress [72, 82-84, 86].

Many gene candidates on the linkage groups of the major and minor QTLs related to cold response were also associated with regulatory genes, including 512 associated with kinases and 159 with transcription factors. Kinases play a crucial role in the regulation of cold tolerance in plants by modulating the activity of various proteins involved in cold acclimation, including phosphorylation of transcription factors, protein regulation, post-translational modification and signal transduction [87]. QCH24 spans a large genetic distance, suggesting that multiple intra-QTL genes are related to chilling injury, and possibly co-localize to a regulatory gene [88].

Many genes related to transport (190), membrane (126) and heat shock (33) proteins were identified across these loci, which could further contribute to the complexity of cold response in basil. In the early stages of cold exposure, transport proteins are up-regulated, such as ATP binding cassettes, sugar and phosphate transporters, as well as ATPases. Over a longer duration, these transporters are down-regulated, whereas genes linked to carbohydrate metabolism are up-regulated [89]. Membrane-related genes are crucial for cold response in plants as they govern the compositional and functional adaptations of the plasma membrane that maintain cellular metabolism, ion homeostasis, and signaling processes, ultimately contributing to cold tolerance or sensitivity [90]. Additionally, chilling temperatures initiate the accumulation of misfolded proteins, which are aided by the chaperone activity of heat shock proteins in stress tolerant plants [91].

Both parents (‘RU Obsession DMR’ and ‘CB15’) are members of the same species (Ocimum basilicum L.). Hybridization produced fertile F1 progeny, and selfing the hybrid produced fertile F2 progeny. Accessions of the same species with no fertility issues in resulting progeny are expected to have the same chromosome numbers. The general consensus is that cultivated sweet basil (Ocimum basilicum L.) is an allotetraploid and therefore genetic mapping and QTL study methods should assume disomic inheritance or “diploidization” [3-10]. However, the prevalence of marker segregation distortion observed in this population challenges these postulations.

It is possible that the marker segregation distortion was because the parents differed in their chromosome numbers. Deviations from the 2n=4x=48 chromosome model have been observed in O. basilicum accessions, especially in those wild collected, with chromosome numbers of 2n=50 [11], 2n=52 [5], 2n=54 [13], 2n=5x=60 [3], 2n=6x=72 [6, 9, 11, 12] and 2n=74 [6]. Digression from this assumption was also observed through construction of the first de novo QTL map of O. basilicum with the identification of 26 linkage groups instead of 12 or even 24 [16]. Furthermore, O. basilicum parents with different chromosome numbers have been crossed and progeny found to be fertile despite the divergent genetic backgrounds [11]. The differing chromosome base numbers between accessions and fertile hybridization suggest a complex evolutionary history of polyploidy and aneuploidy. However, the similarity in nuclear DNA content suggests that the two parents have the same number of chromosomes. Despite the lack of evidence of multivalent formation in O. basilicum [4, 12], segmental allopolyploidy is also a possible explanation for the segregation distortion because tetrasomic inheritance has not yet been explored in basil. Tetraploid rose can exhibit partial preferential pairing, resulting in some genes adopting disomic inheritance and others tetrasomic inheritance [79]. In such cases of segmental allopolyploidy, assuming a disomic model would result in higher ratios of genetic distortion.

Marker segregation distortion could also arise due to a lack of recombination in the F2 progeny. If the parents were too similar in genotype, then there would be few differences in allele calls between the parents and thus a lower level of segregation in the progeny. This explanation would also support reduced recombination levels and lower than expected marker yield.

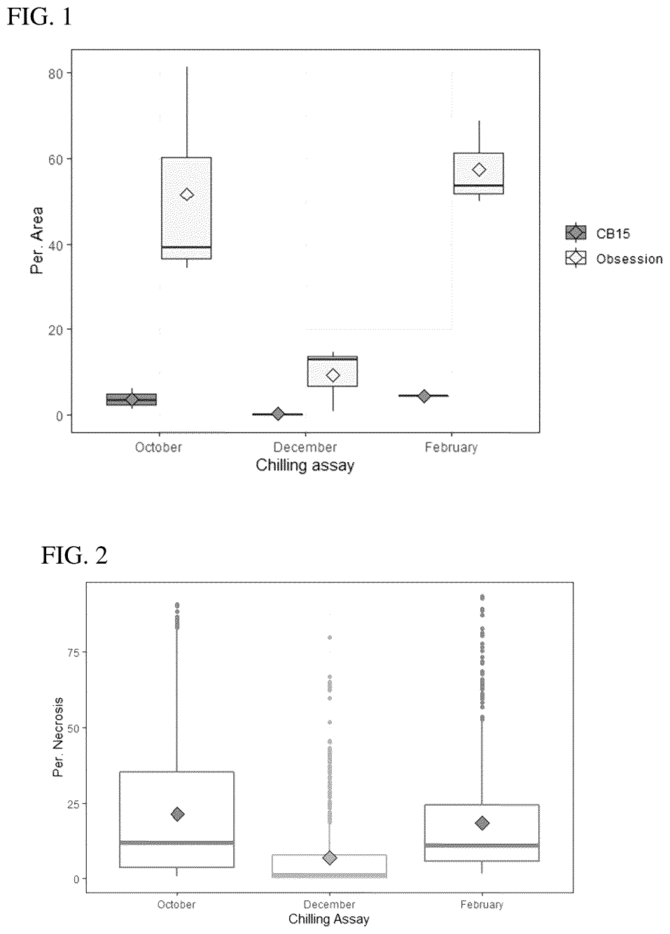

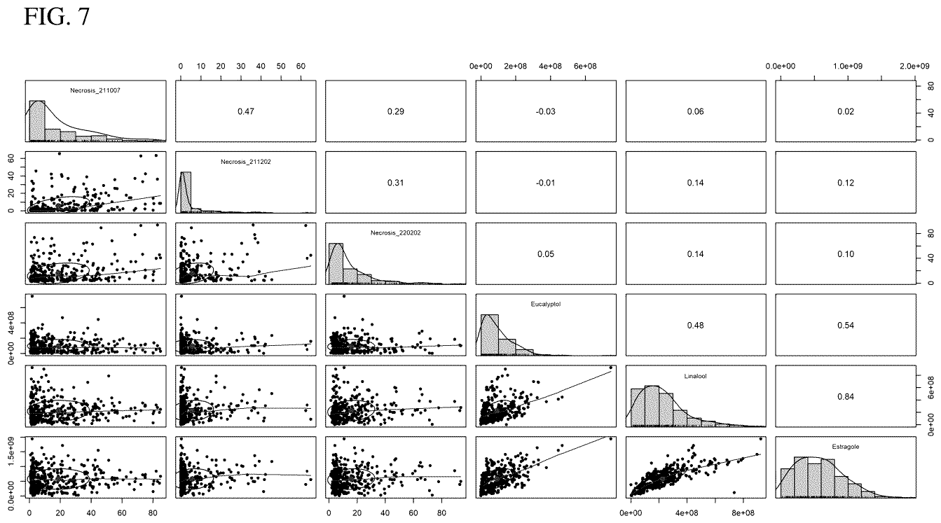

One QTL (QCH24) was detected across all three chilling assay replicates on LG24 spanning a relatively large distance from 0.3 cM up to 78 cM. Four additional QTL were detected in the single-QTL model and two in the MQM. All these QTL were of smaller effect except the QTL on LG16, which had a slightly higher effect than the QTL on LG24 in one replicate. Even though the chilling assays were only moderately correlated, they all detected the same significant QTL (FIGS. 4A-4C and 7). The QTL effect size of LG24 was the largest in the first replicate and was smaller in the second and third replicate. This is likely due to the lower average percent necrosis observed across the entire F2 population in the later replicates (FIG. 2). The population-wide difference in response to chilling temperatures across assays could be due to plant maturation, acclimation to the colder months or photoperiod.

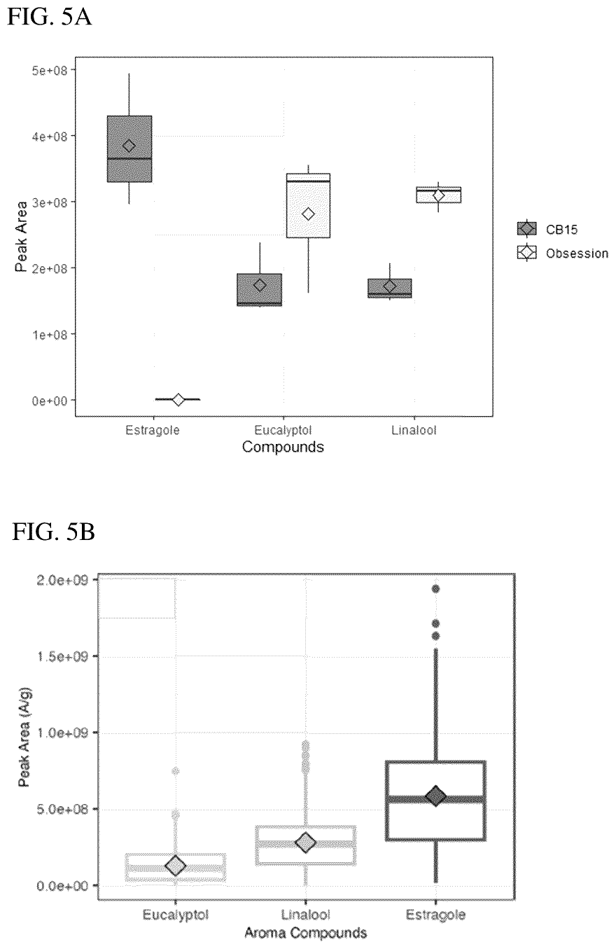

One major QTL (qEST1) was detected for estragole on LG1 spanning from 13.3 to 18.7 cM and for eucalyptol on LG26 (qEUC26) spanning from 113.0 to 132.4 cM. Another smaller effect QTL was detected in the single-QTL model for estragole on LG3, however, it was no longer significant in the MQM. The parents were the least different in their levels of linalool (FIG. 5), which explains why no QTL was detected for this aroma compound.

Estragole, eucalyptol and linalool levels in the F2 population all positively correlated with each other (FIG. 7). Thus, when an F2 individual had a high concentration of one major aroma compound, it also had relatively higher concentrations of the other major aroma compounds. As these compounds belong to different pathways [33-35], common signals may regulate the multiple biosynthetic basil aroma pathways as has been shown in tea [80], especially since both pathways are spatially localized in basil glandular trichomes cells, which can enable direct crosstalk between pathways [81].

The QTLs associated with aroma compounds yielded interesting relationships to potential gene candidates. Two candidate genes were identified as homologous to terpene synthases and one as homologous to terpene cyclase/mutase family members on LG26, which contained the QTL associated with eucalyptol, a terpene (Table 4). No phenylpropenes, propenes or propenoids were identified on LG1, which contained the QTL associated with estragole, a phenylpropene. However, a gene was annotated on LG1 as caffeic acid O-methyltransferase, which is a critical enzyme in the phenylpropanoid metabolic pathway [75]. Caffeoyl-CoA O-methyltransferase is also an important enzyme in phenylpropene synthesis for which 6 annotations were identified on LG1 and LG26 [76].

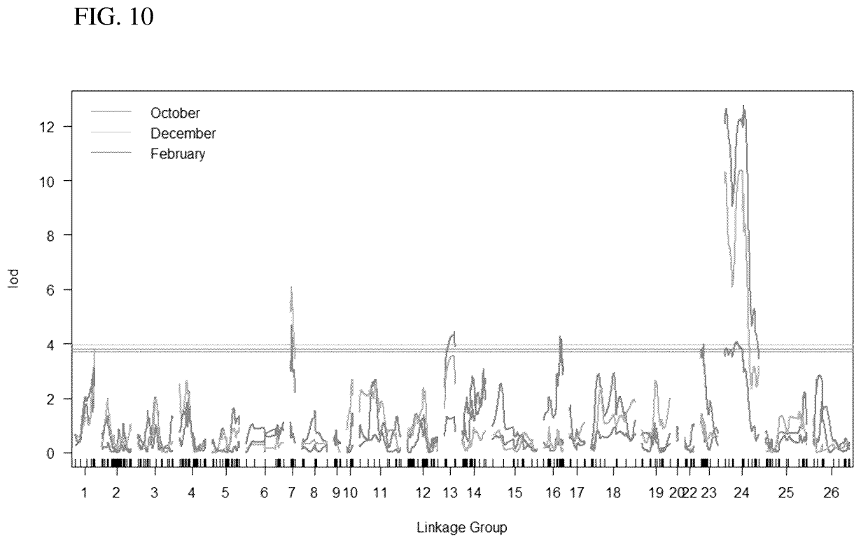

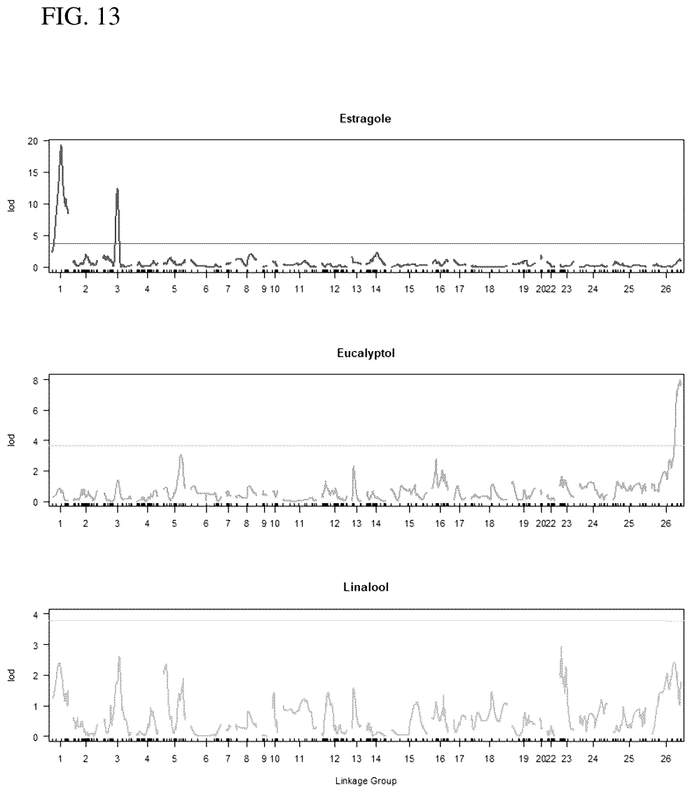

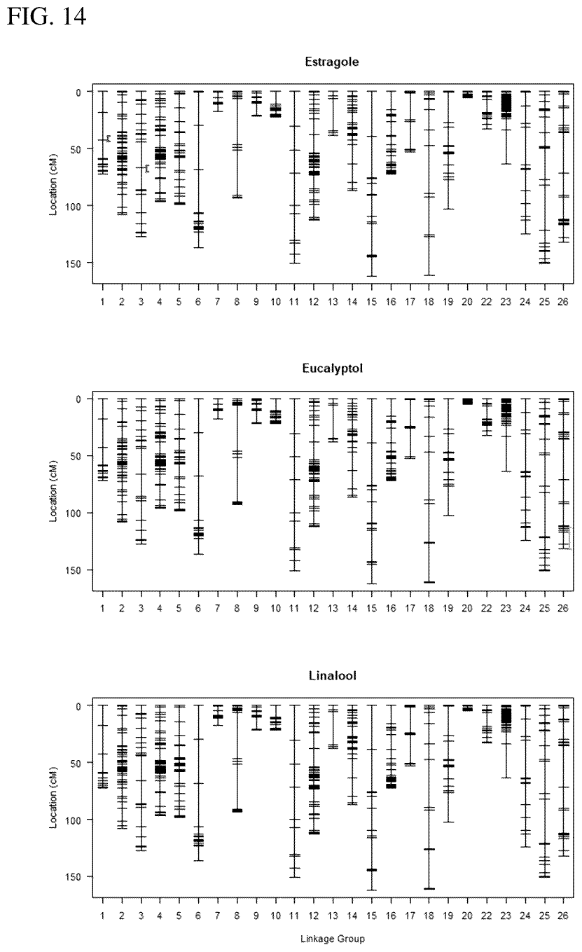

Aroma was evaluated not only for its importance in breeding, but also its potential relationship to chilling tolerance. Previous research observed that when screening sweet basils for chilling tolerance, plants exhibiting chilling tolerance were more associated with licorice aroma in contrast to a more traditional sweet basil aroma [42]. As shown herein, there was no correlation between chilling response in the F2 progeny and the major aroma compounds: estragole, eucalyptol and linalool (FIG. 7). There was also no overlap in the major QTL for chilling injury and aroma compounds (FIGS. 10 and 13). Estragole was of interest as it is responsible for licorice aroma in basil [21]. A QTL on LG1 was significant for estragole and significant in the MQM of two of the chilling assay replicates, however the regions did not overlap and no relevant candidate genes were identified via annotation. Compounds other than those examined in this study may still be associated with chilling response in basil, such as eugenol [92, 93]. Eugenol levels in this population were too low to assess for QTL and correlations with chilling response, but it could play a role in other populations of basil with higher endogenous concentrations.

This application provides chilling tolerant sweet basils, which do not yet exist in the market. A cold-tolerant evergreen hybrid produced from the interspecific cross of O. kilimandscharicum and O. basilicum was reported as being cold tolerant [94]. Although this study did not report the aroma or appearance of the hybrid, it is likely high in camphor and ornamental in appearance like the O. kilimandscharicum parent. This is the case with ‘African blue’ basil, which is an interspecific cross from the same two species (O. kilimandscharicum x O. basilicum ‘Dark Opal’). Furthermore, this variety is sterile, which inhibits commercial seed production and breeding for improved aroma [95] unless embryo rescue or other techniques are used to restore fertility. ‘Magic Mountain’ (Ball Seed) claims to be less cold-sensitive than other basils. However, the aroma and appearance of ‘Magic Mountain’ is not distinctly Genovese-style and ‘CB15’ was significantly more cold tolerant in our evaluations. A newer ornamental basil called ‘Christmas basil’ (Thompson & Morgan; Luv2Garden) has been reported as “half-hardy”, yet this basil is ornamental in appearance with aroma consists of pine, fruit or mulled wine instead of sweet basil.

The present disclosure provides a series of new chilling tolerant basil cultivars (‘OCB-029’, ‘OCB-017’, ‘OCB-022’, ‘OCB-009’, ‘OCB-011’, ‘OCB-027’, ‘OCB-019’, ‘OCB-031’, ‘OCB-121’, ‘OCB-174’, ‘OCB-169’, ‘OCB-153’, ‘OCB-115’, ‘OCB-120’, ‘OCB-119’, ‘OCB-067’, ‘OCB-058’, ‘OCB-086’, ‘OCB-062’, ‘OCB-117’, ‘OCB-057’, ‘OCB-126’, ‘OCB-188’, ‘OCB-129’, and ‘OCB-051’) derived from a series of crosses and selections between, ‘CB15’ and ‘Rutgers Obsession DMR’. The 25 new distinct sweet basil cultivars are each chilling tolerant and some are also tolerant to Peronospora belbahrii, the causal agent of BDM in Ocimum spp. This chilling tolerance, in combination with a sweet basil or Thai basil aroma profile, and in some examples also downy mildew resistance/tolerance, is distinct from all other sweet basils currently on the commercial market. A cold-tolerant evergreen hybrid produced from the interspecific cross of O. kilimandscharicum and O. basilicum was reported as being cold tolerant [94]. Although this study did not report the aroma or appearance of the hybrid, it is likely high in camphor and ornamental in appearance like the O. kilimandscharicum parent, as this is the case with ‘African blue’ basil, which is an interspecific cross from the same species (O. kilimandscharicum x O. basilicum ‘Dark Opal’). Furthermore, this variety is sterile, which inhibits commercial seed production and breeding for improved aroma [95]. ‘Magic Mountain’ (Ball Seed) claims to be less cold-sensitive than other basils, which was supported by chilling assays. However, in indoor greenhouse grown trials, such chilling tolerance was not observed using the methods provided herein. ‘CB15’ was significantly higher in cold tolerance and the aroma and appearance of ‘Magic Mountain’ is not distinctly reminiscent of sweet basil with its purple pigmentation on the young and upper leaves. A newer basil called ‘Christmas basil’ (Thompson & Morgan; Luv2Garden) has been reported as “half-hardy”, yet this basil is still ornamental in appearance and the aroma is of pine, fruit or mulled wine instead of a traditional sweet basil.

This chilling tolerance will allow these varieties to be grown indoors or outdoors, for example where temperatures may get to about <15° C., such as 3-10° C., such as 3-5° C. In addition, this chilling tolerance will allow these varieties to be shipped or stored at lower temperatures, for example temperatures as low as about 3-10° C., such as 3-5° C. Growers, distributors and retailers currently suffer large economic losses due to the sensitivity of all commercial basils to cold spells and shipping conditions. These new varieties mitigate those issues while maintaining and even enhancing other sweet basil attributes.

Selected F2 progeny that resulted from crossing and selection over multiple months were evaluated for chilling tolerance, by evaluating removed leaves stored at about 3-5° C. for about 4-5 days. Plants with leaves having less than 5% necrosis were selected for further evaluation. Repeated evaluations led to the discovery of a series of 25 basil inbred lines having chilling tolerance in addition to the visual appearances, flavor and aromas desired for the traditional sweet basil market. These new cultivars (e.g., seeds, plants, and parts thereof) can be used for the fresh, processing, fresh frozen, ornamental, and heath markets for growers, processors and horticulturalists/home gardeners.

The new sweet basils (‘OCB-029’, ‘OCB-017’, ‘OCB-022’, ‘OCB-009’, ‘OCB-011’, ‘OCB-027’, ‘OCB-019’, ‘OCB-031’, ‘OCB-121’, ‘OCB-174’, ‘OCB-169’, ‘OCB-153’, ‘OCB-115’, ‘OCB-120’, ‘OCB-119’, ‘OCB-067’, ‘OCB-058’, ‘OCB-086’, ‘OCB-062’, ‘OCB-117’, ‘OCB-057’, ‘OCB-126’, ‘OCB-188’, ‘OCB-129’, and ‘OCB-051’) were developed to be chilling tolerant for commercial agricultural production, transportation, storage, and home gardens. These varieties, as well as their progeny, can be grown in the open fields or high tunnels, in greenhouses in pots or other containers, hydroponically, aeroponically, or produced in any manner that when exposed to chilling temperatures, for example as low as about 3-5° C. for 4-5 days, will still produce a marketable fresh or dried or processed product with no-to-few signs/symptoms of cold damage.

The new sweet basil plants also exhibit an aroma and flavor characteristic of sweet basil or Thai basil (see Table 1). Thus, the new sweet basil cultivars ‘OCB-029’, ‘OCB-017’, ‘OCB-022’, ‘OCB-009’, ‘OCB-011’, ‘OCB-027’, ‘OCB-019’, ‘OCB-031’, ‘OCB-121’, ‘OCB-174’, ‘OCB-169’, ‘OCB-153’, ‘OCB-115’, ‘OCB-120’, ‘OCB-119’, ‘OCB-067’, ‘OCB-058’, ‘OCB-086’, ‘OCB-062’, ‘OCB-117’, ‘OCB-057’, ‘OCB-126’, ‘OCB-188’, ‘OCB-129’, and ‘OCB-051’ and progeny thereof can be used in the production of fresh and/or dried sweet basil, or for the distilled aromatic essential oils, which have multiple applications in foods, flavors, fragrances, nutraceuticals, and culinary herbs.

Thus, provided herein are seeds of new basil cultivars ‘OCB-029’, ‘OCB-017’, ‘OCB-022’, ‘OCB-009’, ‘OCB-011’, ‘OCB-027’, ‘OCB-019’, ‘OCB-031’, ‘OCB-121’, ‘OCB-174’, ‘OCB-169’, ‘OCB-153’, ‘OCB-115’, ‘OCB-120’, ‘OCB-119’, ‘OCB-067’, ‘OCB-058’, ‘OCB-086’, ‘OCB-062’, ‘OCB-117’, ‘OCB-057’, ‘OCB-126’, ‘OCB-188’, ‘OCB-129’, and ‘OCB-051’, wherein representative sample seed of the varieties are deposited under ATCC Accession Numbers: ______. The disclosure provides basil plants having or consisting of the morphological and physiological characteristics of the new basil varieties provided herein. The disclosure also provides basil plants having one or more of the morphological and physiological characteristics of the new basils (such as those shown in Table 1). Also provided are seeds of such plants, progeny of such plants, parts of such plants (such as pollen, ovules and cells), and vegetative sprigs or clones of such plants. In one example, the disclosure provides basil plants having the genotype of one or more of the new basils provided herein. For example, the disclosure provides plants produced by growing the seed of a new basil variety provided herein.

The disclosed new basil varieties and seeds can be used to produce other basil plants and seeds, for example as part of a breeding program. Choice of breeding or selection methods using to generate new basil plants and seeds can depend on the mode of plant reproduction, the heritability of the trait(s) being improved, and the type of variety used commercially (e.g., F1 hybrid variety, pure line variety, etc.). For highly heritable traits, a choice of superior individual plants evaluated at a single location can be effective, whereas for traits with low heritability, selection can be based on mean values obtained from replicated evaluations of families of related plants. Exemplary selection methods commonly include pedigree selection, modified pedigree selection, mass selection, recurrent selection and backcrossing.

The complexity of inheritance influences choice of the breeding method. Backcross breeding can be used to transfer one or a few favorable genes for a highly heritable trait into a desirable variety. This approach has been used extensively for breeding disease-resistant varieties (e.g., see Bowers et al., 1992. Crop Sci. 32(1):67-72; Nickell and Bernard, 1992. Crop Sci. 32(3):835). Various recurrent selection techniques can be used to improve quantitatively inherited traits controlled by numerous genes.

Promising advanced breeding lines can be thoroughly tested and compared to appropriate standards in environments representative of the commercial target area(s) for generally two or more years in multiple locations. The best or most preferred lines are candidates for new commercial varieties. Those still deficient in a few traits may be used as parents to produce new populations for further selection.

One method of identifying a superior plant is to observe its performance relative to other experimental plants and to one or more widely grown standard varieties.

Plant breeding can result in new, unique and superior basil varieties and hybrids from a disclosed new basil variety. Two or more parental lines can be selected (such as one or more of new basil cultivars ‘OCB-029’, ‘OCB-017’, ‘OCB-022’, ‘OCB-009’, ‘OCB-011’, ‘OCB-027’, ‘OCB-019’, ‘OCB-031’, ‘OCB-121’, ‘OCB-174’, ‘OCB-169’, ‘OCB-153’, ‘OCB-115’, ‘OCB-120’, ‘OCB-119’, ‘OCB-067’, ‘OCB-058’, ‘OCB-086’, ‘OCB-062’, ‘OCB-117’, ‘OCB-057’, ‘OCB-126’, ‘OCB-188’, ‘OCB-129’, and ‘OCB-051’), followed by repeated selfing and selection, producing many new genetic combinations. Each year, the germplasm to advance to the next generation is selected. This germplasm is grown under unique and different geographical, climatic and soil conditions, and further selections are then made, during and at the end of the growing season.

The development of new basil varieties from new basil cultivars ‘OCB-029’, ‘OCB-017’, ‘OCB-022’, ‘OCB-009’, ‘OCB-011’, ‘OCB-027’, ‘OCB-019’, ‘OCB-031’, ‘OCB-121’, ‘OCB-174’, ‘OCB-169’, ‘OCB-153’, ‘OCB-115’, ‘OCB-120’, ‘OCB-119’, ‘OCB-067’, ‘OCB-058’, ‘OCB-086’, ‘OCB-062’, ‘OCB-117’, ‘OCB-057’, ‘OCB-126’, ‘OCB-188’, ‘OCB-129’, and ‘OCB-051’ involves the development, identification, and selection of promising/interesting basil varieties with desirable traits/characteristics, the continued growing out of those selections and elimination of those not meeting criteria of the plant developer and/or as needed the crossing of these varieties and selection of progeny from the superior hybrid crosses. A hybrid seed is produced by manual crosses between selected male-fertile parents or by using male sterility systems. Hybrids can be identified by using certain single locus traits such as flower color, pubescence color or herbicide resistance which indicate that the seed is truly a hybrid. Additional data on parental lines as well as the phenotype of the hybrid can influence a decision whether to continue with the specific hybrid cross.

Pedigree breeding and recurrent selection breeding methods can be used to develop varieties from breeding populations. Breeding programs combine desirable traits from two or more varieties or various broad-based sources into breeding pools from which varieties are developed by selfing and selection of desired phenotypes. Pedigree breeding is commonly used for the improvement of self-pollinating crops. Two parents (e.g., wherein one of the parents is one of the new varieties provided herein), which possess favorable, complementary traits, are crossed to produce an F1. An F2 population is produced by selfing one or several F1's. Selection of the best or most preferred individuals can begin in the F2 population (or later depending upon the breeding objectives); then, beginning in the F3, the best or most preferred individuals in the best families can be selected. Replicated testing of families can begin in the F3 or F4 generation to improve the effectiveness of selection for traits with low heritability. At an advanced stage of inbreeding (e.g., F6 and F7), the best lines or mixtures of phenotypically similar lines can be tested for potential commercial release as new varieties.

Mass and recurrent selections can be used to improve populations of either self- or cross-pollinating crops. A genetically variable population of heterozygous individuals is either identified or created by intercrossing several different parents. The best or most preferred plants are selected based on individual superiority, outstanding progeny, or excellent combining ability. The selected plants are intercrossed to produce a new population in which further cycles of selection are continued.

Backcross breeding has been used to transfer genetic loci for simply inherited, highly heritable traits into a desirable homozygous variety, which is the recurrent parent (e.g., the new variety disclosed herein). The source of the trait to be transferred is called the donor or nonrecurring parent. The resulting plant is typically expected to have the attributes of the recurrent parent (e.g., variety) and the desirable trait transferred from the donor parent. After the initial cross, individuals possessing the phenotype of the donor parent are selected and repeatedly crossed (backcrossed) to the recurrent parent. The resulting plant is typically expected to have the attributes of the recurrent parent (e.g., variety) and the desirable trait transferred from the donor parent.

The single-seed descent procedure can refer to planting a segregating population, harvesting a sample of one seed per plant, and using the one-seed sample to plant the next generation. When the population has been advanced from the F2 to the desired level of inbreeding, the plants from which lines are derived will each trace to different F2 individuals. The number of plants in a population declines each generation due to failure of some seeds to germinate or some plants to produce at least one seed. As a result, a progeny represents not all of the F2 plants originally sampled in the population when generation advance is completed.

In a multiple-seed procedure, one or more seeds from each plant in a population are commonly harvested and threshed together to form a bulk. Part of the bulk is used to plant the next generation and part is put in reserve. The multiple-seed procedure makes it possible to plant the same number of seeds of a population each generation of inbreeding. Sufficient numbers of seeds are harvested to make up for those plants that did not germinate or produce seed.

Descriptions of other breeding methods that used for different traits and crops can be found in one of several reference books (e.g., Allard. 1960. Principles of plant breeding. Davis, California: John Wiley & Sons, NY, University of California, pp. 50-98; Simmonds. 1979. Principles of crop improvement. New York: Longman, Inc., pp. 369-399; Sneep and Hendriksen. 1979. “Plant breeding perspectives.” Wageningen (ed.), Center for Agricultural Publishing and Documentation; Fehr. 1987). Some combination of the aforementioned breeding strategies can be used to develop varieties from breeding programs.

### Breeding New Basil Varieties with Chilling Tolerance

Methods for crossing one or more of the new basil varieties ‘OCB-029’, ‘OCB-017’, ‘OCB-022’, ‘OCB-009’, ‘OCB-011’, ‘OCB-027’, ‘OCB-019’, ‘OCB-031’, ‘OCB-121’, ‘OCB-174’, ‘OCB-169’, ‘OCB-153’, ‘OCB-115’, ‘OCB-120’, ‘OCB-119’, ‘OCB-067’, ‘OCB-058’, ‘OCB-086’, ‘OCB-062’, ‘OCB-117’, ‘OCB-057’, ‘OCB-126’, ‘OCB-188’, ‘OCB-129’, and ‘OCB-051’ provided herein with itself or a second plant are provided, as are the seeds and plants produced by such methods. Such methods can be used for propagation of a new basil variety provided herein, or can be used to produce hybrid basil seeds and the plants grown therefrom. Hybrid basil plants can be used, for example, in the commercial production of basil products (including leaves, biomass and extracts) or in breeding programs for the production of novel sweet basil varieties. A hybrid plant can also be used as a recurrent parent at any given stage in a backcrossing protocol during the production of a single locus conversion (for example introduction of one or more desirable traits) of a new basil variety provided herein.

Methods of producing basil plants and/or seed are provided. Such methods can include crossing one or more of the new basil varieties ‘OCB-029’, ‘OCB-017’, ‘OCB-022’, ‘OCB-009’, ‘OCB-011’, ‘OCB-027’, ‘OCB-019’, ‘OCB-031’, ‘OCB-121’, ‘OCB-174’, ‘OCB-169’, ‘OCB-153’, ‘OCB-115’, ‘OCB-120’, ‘OCB-119’, ‘OCB-067’, ‘OCB-058’, ‘OCB-086’, ‘OCB-062’, ‘OCB-117’, ‘OCB-057’, ‘OCB-126’, ‘OCB-188’, ‘OCB-129’, and ‘OCB-051’ provided herein with itself or a second basil plant and harvesting a resulting basil seed, such as an F1 hybrid seed. The resulting plant can be grown, resulting in a basil plant or part thereof.

In one example methods of producing an inbred basil plant derived from a new basil variety provided herein (e.g., ‘OCB-029’, ‘OCB-017’, ‘OCB-022’, ‘OCB-009’, ‘OCB-011’, ‘OCB-027’, ‘OCB-019’, ‘OCB-031’, ‘OCB-121’, ‘OCB-174’, ‘OCB-169’, ‘OCB-153’, ‘OCB-115’, ‘OCB-120’, ‘OCB-119’, ‘OCB-067’, ‘OCB-058’, ‘OCB-086’, ‘OCB-062’, ‘OCB-117’, ‘OCB-057’, ‘OCB-126’, ‘OCB-188’, ‘OCB-129’, or ‘OCB-051’) are provided. In one example such methods include (a) generating a progeny plant derived from a new basil variety provided herein by crossing a plant of the new variety with a basil plant of a second variety; (b) crossing the progeny plant with itself or a second plant to produce a seed of a progeny plant of a subsequent generation; (c) growing a progeny plant of a subsequent generation from said seed and crossing the progeny plant of a subsequent generation with itself or a second plant; and (d) repeating steps (b) and (c) for an additional at least 2 generations (such as at least 3, at least 4, at least 5, at least 6, at least 7, at least 8 at least 9, at least 10, at least 15 or at least 20, such as 2 to 10, 3 to 10, or 3 to 15 generations) with sufficient inbreeding to produce an inbred basil plant derived from a new basil variety provided herein.

The second plant crossed with a new sweet basil variety provided herein (e.g., ‘OCB-029’, ‘OCB-017’, ‘OCB-022’, ‘OCB-009’, ‘OCB-011’, ‘OCB-027’, ‘OCB-019’, ‘OCB-031’, ‘OCB-121’, ‘OCB-174’, ‘OCB-169’, ‘OCB-153’, ‘OCB-115’, ‘OCB-120’, ‘OCB-119’, ‘OCB-067’, ‘OCB-058’, ‘OCB-086’, ‘OCB-062’, ‘OCB-117’, ‘OCB-057’, ‘OCB-126’, ‘OCB-188’, ‘OCB-129’, or ‘OCB-051’) for the purpose of developing novel sweet basil varieties, is typically a plant which either themselves exhibit one or more desirable characteristics or which exhibit one or more desired characteristic(s) when in hybrid combination. In one example, the second basil plant is transgenic. Exemplary desired characteristics include, but are not limited to: increased seed yield, increased seedling vigor, modified maturity date, desired plant height, high anthocyanin content, high phenolic content, herbicide tolerance or resistance, drought tolerance or resistance, heat tolerance or resistance, low or high soil pH level tolerance, salt tolerance or resistance, resistance to an insect, resistance to a bacterial disease, resistance to a viral disease, resistance to a fungal disease, resistance to a nematode, resistance to a pest, male sterility, site-specific recombination, abiotic stress tolerance, and increased BDM tolerance and/or resistance. In some examples, the desired characteristics include, but are not limited to ornamental traits such as color, leaf size, and internode length; extended shelf life; and flowering time.

When a new sweet basil variety provided herein is crossed with another different variety, first generation (F1) sweet basil progeny are produced. The hybrid progeny are produced regardless of characteristics of the two varieties produced. As such, an F1 hybrid sweet basil plant can be produced by crossing a sweet basil variety provided herein with any second sweet basil plant. The second sweet basil plant can be genetically homozygous (e.g., inbred) or heterozygous. Therefore, the disclosure provides any F1 hybrid sweet basil plant produced by crossing a new sweet basil variety provided herein with a second sweet basil plant (such as a transgenic plant having one or more genes that confer to the plant one or more desired characteristics).

Basil plants can be crossed by either natural or mechanical techniques. Natural pollination occurs in sweet basil either by self-pollination or natural cross pollination, which typically is aided by pollinating organisms. In either natural or artificial crosses, flowering and flowering time can be a consideration.

Sensitivity to day length can be a consideration when genotypes are grown outside of their area of adaptation. When genotypes adapted to tropical latitudes are grown in the field at higher latitudes, they may not mature before frost occurs. Plants can be induced to flower and mature earlier by creating artificially short days or by grafting. Basil plants can be grown in winter nurseries located at sea level in tropical latitudes where day lengths are shorter than their critical photoperiod. The short day lengths and warm temperatures encourage early flowering and seed maturation. Early flowering can be useful for generation advance when only a few self-pollinated seeds per plant are desired, but usually not for artificial hybridization because the flowers self-pollinate before they are large enough to manipulate for hybridization. Artificial lighting can be used to extend the natural day length to about 14.5 hours to obtain flowers suitable for hybridization and to increase yields of self-pollinated seed. The effect of a short photoperiod on flowering and seed yield can be partly offset by altitude. At tropical latitudes, varieties adapted to the northern U.S. perform more like those adapted to the southern U.S. at high altitudes than they do at sea level. The light level for delay of flowering can be dependent on the quality of light emitted from the source and the genotype being grown. For example, blue light with a wavelength of about 480 nm typically needs more than about 30 times the energy to inhibit flowering as red light with a wavelength of about 640 nm (Parker et al. 1946. Bot. Gaz. 108:1-26).

Temperature can also affect the flowering and development of plants. It can influence the time of flowering and suitability of flowers for hybridization. Artificial hybridization is typically successful between about 26° C. and about 32° C.

Self-pollination can occur naturally in sweet basil with no manipulation of the flowers. In some examples, the crossing of two basil plants is accomplished using artificial hybridization. In artificial hybridization, the flower used as a female in a cross is manually cross pollinated prior to maturation of pollen from the flower, thereby preventing self-fertilization, or alternatively, the male parts of the flower are emasculated using known methods. Exemplary methods for emasculating the male parts of a sweet basil flower include physical removal of the male parts, use of a cytoplasmic or genetic factor conferring male sterility, and application of a chemical gametocide to the male parts.

For artificial hybridization employing emasculation, flowers that are expected to open the following day are selected on the female parent. The buds are swollen and the corolla is just visible through the calyx or has begun to emerge. Usually no more than two buds on a parent plant are prepared, and all self-pollinated flowers or immature buds are removed, for example with forceps. Immature buds, such as those hidden under the stipules at the leaf axil, are removed. The calyx is removed, for example by grasping a sepal with the forceps, pulling it down and around the flower, and repeating the procedure until the five sepals are removed. The exposed corolla is removed, for example by grasping it just above the calyx scar, then lifting and wiggling the forceps simultaneously. The ring of anthers is visible after the corolla is removed, unless the anthers were removed with the petals. Cross-pollination can then be performed using, for example, petri dishes or envelopes in which male flowers have been collected. Desiccators containing calcium chloride crystals are used in some environments to dry male flowers to obtain adequate pollen shed.

Emasculation is not necessary to prevent self-pollination (Walker et al. 1979. Crop Sci. 19:285-286). When emasculation is not used, the anthers near the stigma can be removed to make the stigma visible for pollination. The female flower is usually hand-pollinated immediately after it is prepared; although a delay of several hours does not reduce seed set. Pollen shed typically begins in the morning and can end when temperatures are above about 30° C. Pollen shed can also begin later and continue throughout much of the day with more moderate temperatures.

Pollen is available from a flower with a recently opened corolla, but the degree of corolla opening associated with pollen shed can vary during the day. In many environments, collection and use of male flowers immediately without storage can be conducted. In the southern U.S. and other humid climates, pollen shed occurs in the morning when female flowers are more immature and difficult to manipulate than in the afternoon, and the flowers can be damp from heavy dew. In those circumstances, male flowers are collected into envelopes or petri dishes in the morning, and the open container is typically placed in a desiccator for about 4 hours at a temperature of about 25° C. The desiccator can be taken to the field in the afternoon and kept in the shade to prevent excessive temperatures from developing within it. Pollen viability can be maintained in flowers for up to about 2 days when stored at about 5° C. In a desiccator at about 3° C., flowers can be stored successfully for several weeks; however, varieties can differ in the percentage of pollen that germinates after long-term storage.

Either with or without emasculation of the female flower, hand pollination can be carried out by removing the stamens and pistil from a flower of the male parent and gently brushing the anthers against the stigma of the female flower. Access to the stamens can be achieved by removing the front sepal and keel petals, or piercing the keel with closed forceps and allowing them to open to push the petals away. Brushing the anthers on the stigma causes them to rupture, and high percentages of successful crosses are typically obtained when pollen is clearly visible on the stigma. Pollen shed can be checked by tapping the anthers before brushing the stigma. Several male flowers can be used to obtain suitable pollen shed when conditions are unfavorable, or the same male can be used to pollinate several flowers with good pollen shed.

When male flowers are not collected and dried, the parents of a cross can be planted adjacent to each other. Plants are typically grown in rows about 65 cm to about 100 cm apart. Yield of self-pollinated seed from an individual plant can range from a few seeds to more than about 1,000 as a function of plant density. A density of about 30 plants/m of row can be used when about 30 or fewer seeds per plant is adequate, about 10 plants/m can be used to obtain about 100 seeds/plant, and about 3 plants/m usually results in a high seed production per plant. Densities of about 12 plants/m or less are commonly used for artificial hybridization.

Multiple planting dates about 7 days to about 14 days apart can typically be used to match parents of different flowering dates. When differences in flowering dates are extreme between parents, flowering of the later parent can be hastened by creating an artificially short day. Alternatively, flowering of the earlier parent can be delayed by use of artificially long days or delayed planting. For example, crosses with genotypes adapted to the southern U.S. are made in northern U.S. locations by covering the late genotype with a box, large can, or similar container to create an artificially short photoperiod of about 12 hours for about 15 days beginning when there are three nodes with trifoliate leaves on the main stem. Plants induced to flower early tend to have flowers that self-pollinate when they are small and can be difficult to prepare for hybridization. Grafting can be used to hasten the flowering of late flowering genotypes.

### Basil Plants Having One or More Desired Heritable Traits

The disclosure provides plants of the new sweet basil varieties ‘OCB-029’, ‘OCB-017’, ‘OCB-022’, ‘OCB-009’, ‘OCB-011’, ‘OCB-027’, ‘OCB-019’, ‘OCB-031’, ‘OCB-121’, ‘OCB-174’, ‘OCB-169’, ‘OCB-153’, ‘OCB-115’, ‘OCB-120’, ‘OCB-119’, ‘OCB-067’, ‘OCB-058’, ‘OCB-086’, ‘OCB-062’, ‘OCB-117’, ‘OCB-057’, ‘OCB-126’, ‘OCB-188’, ‘OCB-129’, and ‘OCB-051’, modified to include one or more desired heritable traits. In some examples, such plants can be developed using backcrossing or genetic engineering (for example by introducing one or more transgenes into a disclosed new basil variety ‘OCB-029’, ‘OCB-017’, ‘OCB-022’, ‘OCB-009’, ‘OCB-011’, ‘OCB-027’, ‘OCB-019’, ‘OCB-031’, ‘OCB-121’, ‘OCB-174’, ‘OCB-169’, ‘OCB-153’, ‘OCB-115’, ‘OCB-120’, ‘OCB-119’, ‘OCB-067’, ‘OCB-058’, ‘OCB-086’, ‘OCB-062’, ‘OCB-117’, ‘OCB-057’, ‘OCB-126’, ‘OCB-188’, ‘OCB-129’, and/or ‘OCB-051’, wherein the transgenes encode one or more desired traits), wherein essentially all of the desired morphological and physiological characteristics of a new disclosed sweet basil variety are recovered (such as chilling tolerance, aroma profile, and in some examples also BDM resistance/tolerance) in addition to a genetic locus transferred into the plant via the backcrossing technique. Plants developed using such methods can be referred to as a single locus converted plant.

In one example, the method of introducing one or more desired traits into one or more of the disclosed basil varieties (‘OCB-029’, ‘OCB-017’, ‘OCB-022’, ‘OCB-009’, ‘OCB-011’, ‘OCB-027’, ‘OCB-019’, ‘OCB-031’, ‘OCB-121’, ‘OCB-174’, ‘OCB-169’, ‘OCB-153’, ‘OCB-115’, ‘OCB-120’, ‘OCB-119’, ‘OCB-067’, ‘OCB-058’, ‘OCB-086’, ‘OCB-062’, ‘OCB-117’, ‘OCB-057’, ‘OCB-126’, ‘OCB-188’, ‘OCB-129’, and/or ‘OCB-051’) includes (a) crossing a first plant of sweet basil variety ‘OCB-029’, ‘OCB-017’, ‘OCB-022’, ‘OCB-009’, ‘OCB-011’, ‘OCB-027’, ‘OCB-019’, ‘OCB-031’, ‘OCB-121’, ‘OCB-174’, ‘OCB-169’, ‘OCB-153’, ‘OCB-115’, ‘OCB-120’, ‘OCB-119’, ‘OCB-067’, ‘OCB-058’, ‘OCB-086’, ‘OCB-062’, ‘OCB-117’, ‘OCB-057’, ‘OCB-126’, ‘OCB-188’, ‘OCB-129’, and/or ‘OCB-051’ with a second basil plant (such as a sweet or Thai basil) having one or more desired traits to produce F1 progeny plants; (b) selecting F1 progeny plants that have the one or more desired traits to produce selected F1 progeny plants; (c) crossing the selected progeny plants with at least a first plant of the new variety to produce backcross progeny plants; (d) selecting backcross progeny plants that have the one or more desired traits and physiological and morphological characteristics of a new basil variety to produce selected backcross progeny plants; and (e) repeating steps (c) and (d) one or more times in succession to produce selected second or higher backcross progeny plants that have the one or more desired traits and the physiological and morphological characteristics of a new basil variety ‘OCB-029’, ‘OCB-017’, ‘OCB-022’, ‘OCB-009’, ‘OCB-011’, ‘OCB-027’, ‘OCB-019’, ‘OCB-031’, ‘OCB-121’, ‘OCB-174’, ‘OCB-169’, ‘OCB-153’, ‘OCB-115’, ‘OCB-120’, ‘OCB-119’, ‘OCB-067’, ‘OCB-058’, ‘OCB-086’, ‘OCB-062’, ‘OCB-117’, ‘OCB-057’, ‘OCB-126’, ‘OCB-188’, ‘OCB-129’, and/or ‘OCB-051’ when grown in the same environmental conditions.

Backcrossing methods can be used to improve or introduce a characteristic into basil variety ‘OCB-029’, ‘OCB-017’, ‘OCB-022’, ‘OCB-009’, ‘OCB-011’, ‘OCB-027’, ‘OCB-019’, ‘OCB-031’, ‘OCB-121’, ‘OCB-174’, ‘OCB-169’, ‘OCB-153’, ‘OCB-115’, ‘OCB-120’, ‘OCB-119’, ‘OCB-067’, ‘OCB-058’, ‘OCB-086’, ‘OCB-062’, ‘OCB-117’, ‘OCB-057’, ‘OCB-126’, ‘OCB-188’, ‘OCB-129’, and/or ‘OCB-051’. The parental basil plant, which contributes the locus for the desired characteristic, is termed the “nonrecurring” or “donor” parent. This terminology refers to the fact that the nonrecurring parent is used one time in the backcross protocol and therefore does not recur. The parental basil plant to which the locus or loci from the nonrecurring parent are transferred is known as the recurrent parent as it is used for several rounds in the backcrossing protocol (Poehlman and Sleper. 1995. “Breeding Field Crops” Ames, Iowa: Iowa State University Press; Sprague and Dudley, eds. 1988. Corn and Improvement, 3rd edition). In a typical backcross protocol, the original variety of interest (recurrent parent, e.g., the ‘OCB-029’, ‘OCB-017’, ‘OCB-022’, ‘OCB-009’, ‘OCB-011’, ‘OCB-027’, ‘OCB-019’, ‘OCB-031’, ‘OCB-121’, ‘OCB-174’, ‘OCB-169’, ‘OCB-153’, ‘OCB-115’, ‘OCB-120’, ‘OCB-119’, ‘OCB-067’, ‘OCB-058’, ‘OCB-086’, ‘OCB-062’, ‘OCB-117’, ‘OCB-057’, ‘OCB-126’, ‘OCB-188’, ‘OCB-129’, or ‘OCB-051’ variety disclosed herein) is crossed to a second variety (nonrecurring parent) that carries the single locus of interest (such as a desirable trait) to be transferred. The resulting progeny from this cross are then crossed again to the recurrent parent and the process is repeated until a basil plant is obtained wherein essentially all of the desired morphological and physiological characteristics of the recurrent parent (e.g., the ‘OCB-029’, ‘OCB-017’, ‘OCB-022’, ‘OCB-009’, ‘OCB-011’, ‘OCB-027’, ‘OCB-019’, ‘OCB-031’, ‘OCB-121’, ‘OCB-174’, ‘OCB-169’, ‘OCB-153’, ‘OCB-115’, ‘OCB-120’, ‘OCB-119’, ‘OCB-067’, ‘OCB-058’, ‘OCB-086’, ‘OCB-062’, ‘OCB-117’, ‘OCB-057’, ‘OCB-126’, ‘OCB-188’, ‘OCB-129’, or ‘OCB-051’’ variety disclosed herein) are recovered (such as increased chilling tolerance) in the converted plant, in addition to the single transferred locus from the nonrecurring parent.

A backcross protocol alters or substitutes a single trait or characteristic in the original variety, such as a ‘OCB-029’, ‘OCB-017’, ‘OCB-022’, ‘OCB-009’, ‘OCB-011’, ‘OCB-027’, ‘OCB-019’, ‘OCB-031’, ‘OCB-121’, ‘OCB-174’, ‘OCB-169’, ‘OCB-153’, ‘OCB-115’, ‘OCB-120’, ‘OCB-119’, ‘OCB-067’, ‘OCB-058’, ‘OCB-086’, ‘OCB-062’, ‘OCB-117’, ‘OCB-057’, ‘OCB-126’, ‘OCB-188’, ‘OCB-129’, or ‘OCB-051’ basil variety disclosed herein. To accomplish this, a single locus of the recurrent variety is modified or substituted with the desired locus from the nonrecurrent parent, while retaining essentially all of the rest of the desired genetic, and therefore the desired physiological and morphological constitution of the original variety. The choice of the particular nonrecurrent parent can depend on the purpose of the backcross; for example, a major purpose is to add a commercially desirable, agronomically important trait to the plant. The exact backcrossing protocol can depend on the characteristic or trait being altered to determine an appropriate testing protocol. Although backcrossing methods are simplified when the characteristic being transferred is a dominant allele, a recessive allele can also be transferred. In this instance, it can be useful to introduce a test of the progeny to determine if the desired characteristic has been successfully transferred.

In a backcross where the desired characteristic being transferred to the recurrent parent is controlled by a major gene which can be readily evaluated during the backcrossing, it is common to conduct enough backcrosses to avoid testing individual progeny for specific traits such as yield in extensive replicated tests. In general, four or more backcrosses are used when there is no evaluation of the progeny for specific traits, such as yield. As in this example, lines with the phenotype of the recurrent parent can be composited without the usual replicated tests for traits such as yield, in the individual lines.

Sweet and Thai basil varieties can also be developed from more than two parents, for example using modified backcrossing, which uses different recurrent parents during the backcrossing. Modified backcrossing can be used to replace the original recurrent parent with a variety having certain more desirable characteristics, or multiple parents can be used to obtain different desirable characteristics from each.

Many single locus traits are known that are not regularly selected for in the development of a new inbred but that can be improved by backcrossing techniques. Single locus traits can be, but are not necessarily, transgenic. Examples of these traits include, but are not limited to, male sterility, herbicide resistance, abiotic stress tolerance (such as tolerance or resistance to drought, heat, cold, low or high soil pH level, and/or salt), resistance to bacterial, fungal, or viral disease (such as BDM), insect resistance, restoration of male fertility, enhanced nutritional quality, modified phosphorus characteristics, modified antioxidant characteristics, yield stability, and yield enhancement. These comprise genes generally inherited through the nucleus. Thus plants of a ‘OCB-029’, ‘OCB-017’, ‘OCB-022’, ‘OCB-009’, ‘OCB-011’, ‘OCB-027’, ‘OCB-019’, ‘OCB-031’, ‘OCB-121’, ‘OCB-174’, ‘OCB-169’, ‘OCB-153’, ‘OCB-115’, ‘OCB-120’, ‘OCB-119’, ‘OCB-067’, ‘OCB-058’, ‘OCB-086’, ‘OCB-062’, ‘OCB-117’, ‘OCB-057’, ‘OCB-126’, ‘OCB-188’, ‘OCB-129’, or ‘OCB-051’ basil variety disclosed herein, or progeny thereof, which include a single locus conversion (such as one that confers a desired trait, such as chilling tolerance).

Direct selection can be applied where the single locus acts as a dominant trait. An example of a dominant trait is the herbicide resistance trait (such as glyphosate resistance). For the selection process, the progeny of the initial cross are sprayed with a herbicide (such as RoundUp® herbicide) prior to the backcrossing. The spraying eliminates any plants which do not have the desired herbicide resistance characteristic; only those plants which have the herbicide resistance gene are used in the subsequent backcross. This process is then repeated for all additional backcross generations.

Selection of sweet basil plants for breeding may not be dependent on the phenotype of a plant and instead can be based on genetic investigations. For example, a suitable genetic marker can be used which is genetically-linked to a desired trait. One of these markers can therefore be used to identify the presence or absence of a trait in the offspring of a particular cross, and hence can be used in selection of progeny for continued breeding. This technique is referred to as marker assisted selection. Any other type of genetic marker or other assay which is able to identify the relative presence or absence of a trait of interest in a plant can also be useful for breeding. Procedures for marker assisted selection applicable to plant breeding are well known. Such methods can be useful in the case of recessive traits and variable phenotypes, or where conventional assays are more expensive, time consuming, or otherwise disadvantageous. Types of genetic markers which can be used, but are not limited to, Simple Sequence Length Polymorphisms (SSLPs), Randomly Amplified Polymorphic DNAs (RAPDs), DNA Amplification Fingerprinting (DAF), Sequence Characterized Amplified Regions (SCARs), Arbitrary Primed Polymerase Chain Reaction (AP-PCR), Amplified Fragment Length Polymorphisms (AFLPs) (EP 534 858, which is incorporated herein by reference in its entirety), and Single Nucleotide Polymorphisms (SNPs).

Qualitative characteristics can also be useful as phenotype-based genetic markers in basil; however, some or many may not differ among varieties commonly used as parents. Exemplary genetic markers include flower color, differences in maturity, height, and pest resistance.

Useful or desirable traits can be introduced by backcrossing, as well as directly into a plant by genetic transformation methods. Genetic transformation can therefore be used to insert a selected transgene into the ‘OCB-029’, ‘OCB-017’, ‘OCB-022’, ‘OCB-009’, ‘OCB-011’, ‘OCB-027’, ‘OCB-019’, ‘OCB-031’, ‘OCB-121’, ‘OCB-174’, ‘OCB-169’, ‘OCB-153’, ‘OCB-115’, ‘OCB-120’, ‘OCB-119’, ‘OCB-067’, ‘OCB-058’, ‘OCB-086’, ‘OCB-062’, ‘OCB-117’, ‘OCB-057’, ‘OCB-126’, ‘OCB-188’, ‘OCB-129’, and/or ‘OCB-051’ basil variety disclosed herein or progeny thereof, or can, alternatively, be used for the preparation of transgenes which can be introduced by backcrossing. Thus, the disclosure provides methods of producing a plant of the ‘OCB-029’, ‘OCB-017’, ‘OCB-022’, ‘OCB-009’, ‘OCB-011’, ‘OCB-027’, ‘OCB-019’, ‘OCB-031’, ‘OCB-121’, ‘OCB-174’, ‘OCB-169’, ‘OCB-153’, ‘OCB-115’, ‘OCB-120’, ‘OCB-119’, ‘OCB-067’, ‘OCB-058’, ‘OCB-086’, ‘OCB-062’, ‘OCB-117’, ‘OCB-057’, ‘OCB-126’, ‘OCB-188’, ‘OCB-129’, and/or ‘OCB-051’ basil variety disclosed herein, or progeny thereof, that includes one or more added desired traits, for example that include introducing a transgene(s) conferring the one or more desired traits into the ‘OCB-029’, ‘OCB-017’, ‘OCB-022’, ‘OCB-009’, ‘OCB-011’, ‘OCB-027’, ‘OCB-019’, ‘OCB-031’, ‘OCB-121’, ‘OCB-174’, ‘OCB-169’, ‘OCB-153’, ‘OCB-115’, ‘OCB-120’, ‘OCB-119’, ‘OCB-067’, ‘OCB-058’, ‘OCB-086’, ‘OCB-062’, ‘OCB-117’, ‘OCB-057’, ‘OCB-126’, ‘OCB-188’, ‘OCB-129’, or ‘OCB-051’ basil variety disclosed herein or progeny thereof (for example by transformation with a transgene that confers upon the basil plant the desired trait), thereby producing a plant of the ‘OCB-029’, ‘OCB-017’, ‘OCB-022’, ‘OCB-009’, ‘OCB-011’, ‘OCB-027’, ‘OCB-019’, ‘OCB-031’, ‘OCB-121’, ‘OCB-174’, ‘OCB-169’, ‘OCB-153’, ‘OCB-115’, ‘OCB-120’, ‘OCB-119’, ‘OCB-067’, ‘OCB-058’, ‘OCB-086’, ‘OCB-062’, ‘OCB-117’, ‘OCB-057’, ‘OCB-126’, ‘OCB-188’, ‘OCB-129’, or ‘OCB-051’ basil variety disclosed herein or progeny thereof that includes the one or more added desired traits.

Methods for the transformation of plants, including sweet basil, are known. Methods for introducing a desired nucleic acid molecule (e.g., transgene), such as DNA, RNA, or inhibitory RNAs, which can be employed for the genetic transformation of sweet basil include, but are not limited to, electroporation, microprojectile bombardment, Agrobacterium-mediated transformation and direct DNA uptake by protoplasts or other plant cells.

To effect transformation by electroporation, friable tissues, such as a suspension culture of cells or embryogenic callus, can be used. Alternatively, immature embryos or other organized tissue can be transformed directly. In this technique, the cell walls of target cells can be partially degraded by exposing them to pectin-degrading enzymes (pectolyases) or mechanically wound tissues in a controlled manner. Protoplasts can also be employed for electroporation transformation of plants (Bates. 1994. Mol. Biotechnol. 2(2):135-145; Lazzeri. 1995. Methods Mol. Biol. 49:95-106).

In microprojectile bombardment, particles (such as those comprised of tungsten, platinum, or gold) are coated with nucleic acids and delivered into cells by a propelling force. For the bombardment, cells in suspension are concentrated on filters or solid culture medium. Alternatively, immature embryos or other target cells can be arranged on solid culture medium. The cells to be bombarded are positioned at an appropriate distance below the macroprojectile stopping plate. An exemplary method for delivering DNA into plant cells by acceleration is the Biolistics Particle Delivery System, which can be used to propel particles coated with DNA or cells through a screen, such as a stainless steel or Nytex screen, onto a surface covered with target basil cells. The screen disperses the particles so that they are not delivered to the recipient cells in large aggregates. A screen intervening between the projectile apparatus and the cells to be bombarded can reduce the size of projectiles aggregate and contribute to a higher frequency of transformation by reducing the damage inflicted on the recipient cells by projectiles that are too large.

Agrobacterium-mediated transfer is a method for introducing gene loci into plant cells. DNA can be introduced into whole plant tissues, thereby bypassing the need for regeneration of an intact plant from a protoplast. Agrobacterium transformation vectors are capable of replication in E. coli as well as Agrobacterium, allowing for convenient manipulations (Klee et al. 1985. Bio. Tech. 3(7):637-342). Moreover, vectors for Agrobacterium-mediated gene transfer have improved the arrangement of genes and restriction sites in the vectors to facilitate the construction of vectors capable of expressing various polypeptide coding genes. Such vectors have convenient multi-linker regions flanked by a promoter and a polyadenylation site for direct expression of inserted polypeptide coding genes. Additionally, Agrobacterium containing both armed and disarmed Ti genes can be used for transformation. The use of Agrobacterium-mediated plant integrating vectors to introduce DNA into plant cells is known (e.g., Fraley et al. 1985. Bio. Tech. 3(7):629-635; U.S. Pat. No. 5,563,055). Briefly, plant tissue (often leaves) is cut into small pieces, e.g. 10 mm×10 mm, and soaked for 10 minutes in a fluid containing suspended Agrobacterium. Some cells along the cut will be transformed by the bacterium, which inserts its DNA into the cell, which is placed on selectable rooting and shooting media, allowing the plants to regrow. Some plants can be transformed just by dipping the flowers into suspension of Agrobacterium and then planting the seeds in a selective medium. Basil has been transformed using Agrobacterium (Dechamps and Simon. 2002. Plant Cell Reports 21:359-364).

Transformation of plant protoplasts can also be achieved using methods based on calcium phosphate precipitation, polyethylene glycol treatment, electroporation, and combinations of these treatments (e.g., Potrykus et al. 1985. Mol. Gen. Genet. 199(2):169-177; Omirulleh et al. 1993. Plant Mol. Biol. 21(3):415-428; Fromm et al. 1986. Nature. 319(6056):791-739; Uchimiya et al. 1986. Mol. Gen. Genet. 204(2):207-207; Marcotte et al. 1988. Nature 335(6189):454-457).

In one example, such methods can also be used to introduce transgenes for the production of proteins in transgenic basil cells. The resulting produced protein can be harvested from the transgenic basil. The transgene can be harvested from the transgenic plants that are originated or are descended from the new sweet basil variety disclosed herein, a seed of such a basil or a hybrid progeny of such a basil.

Numerous different genes are known and can be introduced into the ‘OCB-029’, ‘OCB-017’, ‘OCB-022’, ‘OCB-009’, ‘OCB-011’, ‘OCB-027’, ‘OCB-019’, ‘OCB-031’, ‘OCB-121’, ‘OCB-174’, ‘OCB-169’, ‘OCB-153’, ‘OCB-115’, ‘OCB-120’, ‘OCB-119’, ‘OCB-067’, ‘OCB-058’, ‘OCB-086’, ‘OCB-062’, ‘OCB-117’, ‘OCB-057’, ‘OCB-126’, ‘OCB-188’, ‘OCB-129’, and/or ‘OCB-051’ basil variety disclosed herein, or progeny thereof. Non-limiting examples of particular genes and corresponding phenotypes that can be chosen for introduction into a basil plant are provided herein.

### Herbicide Resistance

One or more herbicide resistance genes can be used with the methods and plants provided herein. In some examples, a ‘OCB-029’, ‘OCB-017’, ‘OCB-022’, ‘OCB-009’, ‘OCB-011’, ‘OCB-027’, ‘OCB-019’, ‘OCB-031’, ‘OCB-121’, ‘OCB-174’, ‘OCB-169’, ‘OCB-153’, ‘OCB-115’, ‘OCB-120’, ‘OCB-119’, ‘OCB-067’, ‘OCB-058’, ‘OCB-086’, ‘OCB-062’, ‘OCB-117’, ‘OCB-057’, ‘OCB-126’, ‘OCB-188’, ‘OCB-129’, and/or ‘OCB-051’ basil variety disclosed herein includes a gene(s) that confers herbicide resistance. In particular examples, one or more herbicide resistance genes confer tolerance to an herbicide comprising glyphosate, sulfonylurea, imidazalinone, dicamba, glufosinate, phenoxy proprionic acid, cyclohexone, triazine, benzonitrile, broxynil, L-phosphinothricin, cyclohexanedione, chlorophenoxy acetic acid, or combinations thereof.

In one example the herbicide resistance gene is a gene that confers resistance to an herbicide that inhibits the growing point or meristem, such as an imidazalinone or a sulfonylurea. Exemplary genes in this category code for mutant ALS and AHAS enzyme as described, for example, by Lee et al. (1988. Embryo J. 7:1241-8) and Miki et al. (1990. Theoret. Appl. Genet. 80:449-458).

Resistance genes for glyphosate (resistance conferred by mutant 5-enolpyruvl-3 phosphikimate synthase (EPSP) and aroA genes, respectively) and other phosphono compounds such as glufosinate (phosphinothricin acetyl transferase (PAT) and Streptomyces hygroscopicus phosphinothricin-acetyl transferase (bar) genes) can be used (e.g., see U.S. Pat. No. 4,940,835). Examples of specific EPSPS transformation events conferring glyphosate resistance are described, for example, in U.S. Pat. No. 6,040,497.

DNA molecules encoding a mutant aroA gene are known (e.g., ATCC accession number 39256 and U.S. Pat. No. 4,769,061), as are sequences for glutamine synthetase genes, which confer resistance to herbicides such as L-phosphinothricin (e.g., U.S. Pat. No. 4,975,374), phosphinothricin-acetyltransferase (e.g., U.S. Pat. No. 5,879,903). DeGreef et al. (1989. Bio/Technology 61-64) describe the production of transgenic plants that express chimeric bar genes coding for phosphinothricin acetyl transferase activity. Exemplary genes conferring resistance to phenoxy propionic acids and cyclohexones, such as sethoxydim and haloxyfop are the Acct-S1, Accl-S2 and Acct-S3 genes described by Marshall et al. (1992. Theor Appl Genet. 83:435-442).

Exemplary genes conferring resistance to an herbicide that inhibits photosynthesis include triazine (psbA and gs+ genes) and benzonitrile (nitrilase gene) (see Przibilla et al., 1991. Plant Cell. 3:169-174). Nucleotide sequences for nitrilase genes are disclosed in U.S. Pat. No. 4,810,648, and DNA molecules containing these genes are available under ATCC Accession Nos. 53435, 67441, and 67442. Cloning and expression of DNA coding for a glutathione S-transferase is described by Hayes et al. (1992. Biochem. J. 285:173).

U.S. Patent Publication No: 20030135879 describes dicamba monooxygenase (DMO) from Pseuodmonas maltophilia, which is involved in the conversion of a herbicidal form of the herbicide dicamba to a non-toxic 3,6-dichlorosalicylic acid and thus can be used for producing plants tolerant to this herbicide.

The metabolism of chlorophenoxyacetic acids, such as, for example 2,4-D herbicide, is well known. Genes or plasmids that contribute to the metabolism of such compounds are described, for example, by Muller et al. (2006. Appl. Environ. Microbiol. 72(7):4853-4861), Don and Pemberton (1981. J Bacteriol 145(2):681-686), Don et al. (1985. J Bacteriol 161(1):85-90) and Evans et al. (1971. Biochem J 122(4):543-551).

### Disease Resistance

In some examples, a ‘OCB-029’, ‘OCB-017’, ‘OCB-022’, ‘OCB-009’, ‘OCB-011’, ‘OCB-027’, ‘OCB-019’, ‘OCB-031’, ‘OCB-121’, ‘OCB-174’, ‘OCB-169’, ‘OCB-153’, ‘OCB-115’, ‘OCB-120’, ‘OCB-119’, ‘OCB-067’, ‘OCB-058’, ‘OCB-086’, ‘OCB-062’, ‘OCB-117’, ‘OCB-057’, ‘OCB-126’, ‘OCB-188’, ‘OCB-129’, and/or ‘OCB-051’ basil variety disclosed herein includes a gene(s) that confers resistance to one or more diseases. Plant defenses are often activated by specific interaction between the product of a disease resistance gene (R) in the plant and the product of a corresponding avirulence (Avr) gene in the pathogen. A plant, such as the ‘OCB-029’, ‘OCB-017’, ‘OCB-022’, ‘OCB-009’, ‘OCB-011’, ‘OCB-027’, ‘OCB-019’, ‘OCB-031’, ‘OCB-121’, ‘OCB-174’, ‘OCB-169’, ‘OCB-153’, ‘OCB-115’, ‘OCB-120’, ‘OCB-119’, ‘OCB-067’, ‘OCB-058’, ‘OCB-086’, ‘OCB-062’, ‘OCB-117’, ‘OCB-057’, ‘OCB-126’, ‘OCB-188’, ‘OCB-129’, and ‘OCB-051’ basil variety disclosed herein or progeny thereof, can be transformed with cloned resistance gene to engineer plants that are resistant to specific pathogen strains. See, for example Jones et al. (1994. Science 266:789) (tomato Cf-9 gene for resistance to Cladosporium fulvum); Martin et al. (1993. Science 262(5138):1432-1436) (tomato Pto gene for resistance to Pseudomonas syringae pv.); and Mindrinos et al. (1994. Cell 78:1089-1099) (Arabidopsis RSP2 gene for resistance to Pseudomonas syringae).

A viral-invasive protein or a complex toxin derived therefrom can also be used for viral disease resistance. For example, the accumulation of viral coat proteins in transformed plant cells imparts resistance to viral infection and/or disease development effected by the virus from which the coat protein gene is derived, as well as by related viruses. See Beachy et al. (1990. Annu Rev Phytopathol 28:451-474). Coat protein-mediated resistance has been conferred upon transformed plants against alfalfa mosaic virus, cucumber mosaic virus, tobacco streak virus, potato virus X, potato virus Y, tobacco etch virus, tobacco rattle virus and tobacco mosaic virus. Id.

A virus-specific antibody can also be used. See, for example, Tavladoraki et al. (1993. Nature 366:469-472), which shows that transgenic plants expressing recombinant antibody genes are protected from virus attack.

Logemann et al. (1992. Bio/Technology 10:305-308) disclose transgenic plants expressing a barley ribosome-inactivating gene have an increased resistance to fungal disease.

### Insect Resistance

In some examples, a ‘OCB-029’, ‘OCB-017’, ‘OCB-022’, ‘OCB-009’, ‘OCB-011’, ‘OCB-027’, ‘OCB-019’, ‘OCB-031’, ‘OCB-121’, ‘OCB-174’, ‘OCB-169’, ‘OCB-153’, ‘OCB-115’, ‘OCB-120’, ‘OCB-119’, ‘OCB-067’, ‘OCB-058’, ‘OCB-086’, ‘OCB-062’, ‘OCB-117’, ‘OCB-057’, ‘OCB-126’, ‘OCB-188’, ‘OCB-129’, and/or ‘OCB-051’ basil variety disclosed herein includes a gene(s) that confers resistance to one or more insects. One example of an insect resistance gene includes a Bacillus thuringiensis (Bt) protein, a derivative thereof or a synthetic polypeptide modeled thereon (e.g., see Geiser et al., 1986. Gene 48:109, discloses a Bt Δendotoxin gene). Moreover, DNA molecules encoding Δ-endotoxin genes can be obtained from the ATCC (Manassas, VA), for example under ATCC Accession Nos. 40098, 67136, 31995 and 31998. Another example is a lectin. See, for example, Van Damme et al. (1994. Plant Mol Biol 24(5):825-830), which discloses several Clivia miniata mannose-binding lectin genes. A vitamin-binding protein can also be used, such as avidin. See WIPO Publication No. WO 1994/000992, which teaches the use of avidin and avidin homologues as larvicides against insect pests.

In one example the insect resistance gene is an enzyme inhibitor, for example, a protease, proteinase inhibitor, or an α-amylase inhibitor. See, for example, Abe et al. (1987. J. Biol. Chem. 262:16793-7; discloses a rice cysteine proteinase inhibitor), Genbank Accession Nos. Z99173.1 and DQ009797.1 which disclose proteinase inhibitor coding sequences, and Sumitani et al. (1993. Plant Mol. Biol. 21:985; discloses Streptomyces nitrosporeus α-amylase inhibitor). An insect-specific hormone or pheromone can also be used. See, for example, Hammock et al. (1990. Nature 344:458-461; discloses juvenile hormone esterase, an inactivator of juvenile hormone).

Still other examples include an insect-specific antibody or an immunotoxin derived therefrom and a developmental-arrestive protein. See Taylor et al. (1994. Seventh Intl. Symposium on Molecular Plant-Microbe Interactions (Edinburgh Scotland), Abstract #497), who described enzymatic inactivation in transgenic tobacco via production of single-chain antibody fragments.

### Male Sterility

In some examples, a ‘OCB-029’, ‘OCB-017’, ‘OCB-022’, ‘OCB-009’, ‘OCB-011’, ‘OCB-027’, ‘OCB-019’, ‘OCB-031’, ‘OCB-121’, ‘OCB-174’, ‘OCB-169’, ‘OCB-153’, ‘OCB-115’, ‘OCB-120’, ‘OCB-119’, ‘OCB-067’, ‘OCB-058’, ‘OCB-086’, ‘OCB-062’, ‘OCB-117’, ‘OCB-057’, ‘OCB-126’, ‘OCB-188’, ‘OCB-129’, and/or ‘OCB-051’ basil variety disclosed herein includes a gene(s) that confers male sterility. Genetic male sterility can increase the efficiency with which hybrids are made, in that it can eliminate the need to physically emasculate the basil plant used as a female in a given cross (Brim and Stuber. 1973. Crop Sci. 13:528-530). Herbicide-inducible male sterility systems are known (e.g., U.S. Pat. No. 6,762,344).

Where use of male-sterility systems is desired, it can be beneficial to also utilize one or more male-fertility restorer genes. For example, where cytoplasmic male sterility (CMS) is used, hybrid seed production involves three inbred lines: (1) a cytoplasmically male-sterile line having a CMS cytoplasm; (2) a fertile inbred with normal cytoplasm, which is isogenic with the CMS line for nuclear genes (“maintainer line”); and (3) a distinct, fertile inbred with normal cytoplasm, carrying a fertility restoring gene (“restorer” line). The CMS line is propagated by pollination with the maintainer line, with all of the progeny being male sterile, as the CMS cytoplasm is derived from the female parent. These male sterile plants can then be efficiently employed as the female parent in hybrid crosses with the restorer line, without the need for physical emasculation of the male reproductive parts of the female parent.

The presence of a male-fertility restorer gene results in the production of fully fertile F1 hybrid progeny. If no restorer gene is present in the male parent, male-sterile hybrids are obtained. Such hybrids are useful where the vegetative tissue of the basil plant is utilized. However, in many cases, the seeds are considered to be a valuable portion of the crop, thus, it is desirable to restore the fertility of the hybrids in these crops. Therefore, the disclosure provides plants of the ‘OCB-029’, ‘OCB-017’, ‘OCB-022’, ‘OCB-009’, ‘OCB-011’, ‘OCB-027’, ‘OCB-019’, ‘OCB-031’, ‘OCB-121’, ‘OCB-174’, ‘OCB-169’, ‘OCB-153’, ‘OCB-115’, ‘OCB-120’, ‘OCB-119’, ‘OCB-067’, ‘OCB-058’, ‘OCB-086’, ‘OCB-062’, ‘OCB-117’, ‘OCB-057’, ‘OCB-126’, ‘OCB-188’, ‘OCB-129’, and ‘OCB-051’ basil variety disclosed herein comprising a genetic locus capable of restoring male fertility in an otherwise male-sterile plant. Examples of male-sterility genes and corresponding restorers which can be employed are known (see, e.g., U.S. Pat. Nos. 5,530,191 and 5,684,242).

### Exemplary Gene Editing Methods

A gene editing system can be used to manipulate gene expression in plants. For example, basil plants herein can be edited to introduce one or more desired traits. In some examples, a transgene is introduced. In some examples, an existing gene is edited.

In some examples a gene editing system is used that includes one or more nucleic acid (e.g., DNA or RNA)-binding domains or components and one or more nucleic acid (e.g., DNA or RNA)-modifying domains or components, or isolated nucleic acids, e.g., one or more vectors, encoding said nucleic acid (e.g., DNA or RNA)-binding and nucleic acid (e.g., DNA or RNA)-modifying domains or components. Gene editing systems can be used for modifying a coding sequence of a target gene and/or for modulating the expression of a target gene, e.g., by modifying a non-coding/regulatory sequence (e.g., operator or promoter) of the gene, or by modifying the coding sequence/expression of a regulator (e.g., repressor or activator) of the gene. In some examples, the one or more nucleic acid (e.g., DNA or RNA)-binding domains or components are associated with the one or more nucleic acid (e.g., DNA or RNA)-modifying domains or components, such that the one or more nucleic acid (e.g., DNA or RNA)-binding domains target the one or more nucleic acid (e.g., DNA or RNA)-modifying domains or components to a specific nucleic acid site. Methods and compositions for enhancing gene editing is known. See example, U.S. Patent Application Publication No. 2018/0245065. The one or more nucleic acid (e.g., DNA or RNA)-binding domains can be protein domains or nucleic acids that are engineered to recognize target sequences.

Exemplary gene editing systems include but are not limited to, zinc finger nucleases (ZFNs), transcription activator-like effector nucleases (TALENs), CRISPR/Cas systems, meganuclease systems, Fok1 restriction endonuclease systems, and viral vector-mediated gene editing. In some embodiments, CRISPR/Cas-based gene editing methods are used to genetically modify the genome of a basil of the present disclosure.

### A. CRISPR/Cas Systems

CRISPR and Cas were originally discovered as adaptive immunity systems evolved by bacteria and archaea to protect against viral and plasmid invasion. Naturally occurring CRISPR/Cas systems in bacteria are composed of one or more Cas genes and one or more CRISPR arrays consisting of short palindromic repeats of base sequences separated by genome-targeting sequences acquired from previously encountered viruses and plasmids (called spacers) (Wiedenheft, B., et. al. Nature. 2012; 482:331; Bhaya, D., et. al., Annu. Rev. Genet. 2011; 45:231; and Terms, M. P. et. al., Curr. Opin. Microbiol. 2011; 14:321). Bacteria and archaea possessing one or more CRISPR loci respond to viral or plasmid challenge by integrating short fragments of foreign sequence (protospacers) into the host chromosome at the proximal end of the CRISPR array. Transcription of CRISPR loci generates a library of CRISPR-derived RNAs (crRNAs) containing sequences complementary to previously encountered invading nucleic acids (Haurwitz, R. E., et. Al., Science. 2012:329; 1355; Gesner, E. M., et. Al., Nat. Struct. Mol. Biol. 2001, 18:688; Jinek, M., et. Al., Science. 2012:337; 816-21). Target recognition by crRNAs occurs through complementary base pairing with target DNA, which directs cleavage of foreign sequences by means of Cas proteins (Jinek et. Al. 2012 “A Programmable dual-RNA-guided DNA endonuclease in adaptive bacterial immunity.” Science. 2012:337; 816-821).

There are at least five main CRISPR system types (Type I, II, III, IV and V) and at least 16 distinct subtypes (Makarova, K. S., et al., Nat Rev Microbiol. 2015. Nat. Rev. Microbiol. 13, 722-736). CRISPR systems are also classified based on their effector proteins. Class 1 systems possess multi-subunit crRNA-effector complexes, whereas in Class 2 systems all functions of the effector complex are carried out by a single protein (e.g., Cas9 or Cpf1). In some embodiments, the present disclosure provides using type II and/or type V single-subunit effector systems.

As these naturally occur in many different types of bacteria, the exact arrangements and structures of CRISPR, function and number of Cas genes and their product differ somewhat from species to species (Haft et al. (2005) PloS Comput. Biol. 1: e60; Kunin et al. (2007) Genome Biol. 8: R61; Mojica et al. (2005) J. Mol. Evol. 60: 174-182; Bolotin et al. (2005) Microbiol. 151: 2551-2561; Pourcel et al. (2005) Microbiol. 151: 653-663; and Stern et al. (2010) Trends. Genet. 28: 335-340.) For example, the Cas (Cas subtype, E. coli) proteins (e.g., CasA) form a functional complex, Cascade, which processes CRISPR RNA transcripts into spacer-repeat units that Cascade retains (Brouns et al. (2008) Science 321: 960-964). In other prokaryotes, Cas6 processes the CRISPR transcript. The CRISPR-based phage inactivation in E. coli requires Cascade and Cas3, but not Cas1 or Cas2. The Cmr (Cas RAMP module) proteins in Pyrococcus furiosus and other prokaryotes form a functional complex with small CRISPR RNAs that recognizes and cleaves complementary target RNAs. A simpler CRISPR system relies on the protein Cas9, which is a nuclease with two active cutting sites, one for each strand of the double helix. Combining Cas9 and modified CRISPR locus RNA can be used in a system for gene editing (Pennisi (2013) Science 341: 833-836).

### B. CRISPR/Cas9

Provided are methods of gene editing using a Type II CRISPR system. Type II systems rely on i) a single endonuclease protein, ii) a transactivating crRNA (tracrRNA), and iii) a crRNA wherein a ˜20-nucleotide (nt) portion of the 5′ end of the crRNA is complementary to a target nucleic acid. The region of a CRISPR crRNA strand that is complementary to its target DNA protospacer is referred to as “guide sequence.”

In some embodiments, the tracrRNA and crRNA components of a Type II system can be replaced by a single guide RNA (sgRNA), also known as a guide RNA (gRNA). The sgRNA can include, for example, a nucleotide sequence that comprises an at least 12-20 nucleotide sequence complementary to the target DNA sequence (guide sequence) and can include a common scaffold RNA sequence at its 3′ end. As used herein, “a common scaffold RNA” refers to any RNA sequence that mimics the tracrRNA sequence or any RNA sequences that function as a tracrRNA.

Cas9 endonucleases produce blunt end DNA breaks, and are recruited to target DNA by a combination of a crRNA oligo and a tracrRNA oligo, which tether the endonuclease via complementary hybridization of the RNA CRISPR complex.

In some embodiments, DNA recognition by the crRNA/endonuclease complex uses additional complementary base-pairing with a protospacer adjacent motif (PAM) (e.g., 5′-NGG-3′) located in a 3′ portion of the target DNA, downstream from the target protospacer (Jinek, M., et. Al., Science. 2012, 337:816-821). In some embodiments, the PAM motif recognized by a Cas9 varies for different Cas9 proteins.

In some embodiments, the Cas9 disclosed herein can be any variant derived or isolated from any source. In other embodiments, the Cas9 peptide of the present disclosure can include one or more of the mutations described in the literature, including but not limited to the functional mutations described in: Fonfara et al. Nucleic Acids Res. 2014 February; 42(4):2577-90; Nishimasu H. et al. Cell. 2014 Feb. 27,156(5):935-49; Jinek M. et al. Science. 2012 337:816-21; and Jinek M. et al. Science. 2014 Mar. 14, 343(6176). See also U.S. patent application Ser. No. 13/842,859, filed Mar. 15, 2013, which is hereby incorporated by reference; further, see U.S. Pat. Nos. 8,697,359; 8,771,945; 8,795,965; 8,865,406; 8,871,445; 8,889,356; 8,895,308; 8,906,616; 8,932,814; 8,945,839; 8,993,233; and 8,999,641. Thus, in some embodiments, the systems and methods disclosed herein can be used with the wild type Cas9 protein having double-stranded nuclease activity, Cas9 mutants that act as single stranded nickases, or other mutants with modified nuclease activity.

Cas9 molecules of, derived from, or based on the Cas9 proteins of a variety of species can be used in the methods and compositions described herein. For example, Cas9 molecules of, derived from, or based on, e.g., S. pyogenes, S. thermophilus, Staphylococcus aureus and/or Neisseria meningitidis Cas9 molecules, can be used in the systems, methods and compositions described herein. Additional Cas9 species include those from: Acidovorax avenae, Actinobacillus pleuropneumoniae, Actinobacillus succinogenes, Actinobacillus suis, Actinomyces sp., cycliphilus denitrificans, Aminomonas paucivorans, Bacillus cereus, Bacillus smithii, Bacillus thuringiensis, Bacteroides sp., Blastopirellula marina, Bradyrhiz obium sp., Brevibacillus latemsporus, Campylobacter coli, Campylobacter jejuni, Campylobacter lad, Candidatus Puniceispirillum, Clostridiu cellulolyticum, Clostridium perfringens, Corynebacterium accolens, Corynebacterium diphtheria, Corynebacterium matruchotii, Dinoroseobacter sliibae, Eubacterium dolichum, gamma proteobacterium, Gluconacetobacler diazotrophicus, Haemophilus parainfluenzae, Haemophilus sputorum, Helicobacter canadensis, Helicobacter cinaedi, Helicobacter mustelae, Ilyobacler polytropus, Kingella kingae, Lactobacillus crispatus, Listeria ivanovii, Listeria monocytogenes, Listeriaceae bacterium, Methylocystis sp., Methylosinus trichosporium, Mobiluncus mulieris, Neisseria bacilliformis, Neisseria cinerea, Neisseria flavescens, Neisseria lactamica, Neisseria sp., Neisseria wadsworthii, Nitrosomonas sp., Parvibaculum lavamentivorans, Pasteurella multocida, Phascolarctobacterium succinatutens, Ralstonia syzygii, Rhodopseudomonas palustris, Rhodovulum sp., Simonsiella muelleri, Sphingomonas sp., Sporolactobacillus vineae, Staphylococcus lugdunensis, Streptococcus sp., Subdoligranulum sp., Tislrella mobilis, Treponema sp., or Verminephrobacter eiseniae.

In some embodiments, provided are tools for genome editing techniques in plants such as crops and methods of gene editing using Cas endonucleases including SpyCas9, SaCas9, and St1Cas9. See example, Song et al. (2016), The Crop Journal 4:75-82, Mali et al. (2013) Science 339: 823-826; Ran et al. (2015) Nature 520: 186-191; Esvelt et al. (2013) Nature methods 10(11): 1116-1121.

### C. CRISPR/Cpf1

In some embodiments, the present disclosure provides methods of gene editing using a Type V CRISPR system. In some embodiments, the present disclosure provides methods of gene editing using CRISPR from Prevotella, Francisella, Acidaminococcus, Lachnospiraceae, or Moraxella (Cpf1).

The Cpf1 CRISPR systems of the present disclosure can include i) a single endonuclease protein, and ii) a crRNA, wherein a portion of the 3′ end of crRNA contains the guide sequence complementary to a target nucleic acid. In this system, the Cpf1 nuclease is directly recruited to the target DNA by the crRNA. In some embodiments, guide sequences for Cpf1 are at least 12 nt, 13 nt, 14 nt, 15 nt, or 16 nt in order to achieve detectable DNA cleavage, and a minimum of 14 nt, 15 nt, 16 nt, 17 nt, or 18 nt to achieve efficient DNA cleavage.

The Cpf1 systems differ from Cas9 in a variety of ways. First, unlike Cas9, Cpf1 does not require a separate tracrRNA for cleavage. In some embodiments, Cpf1 crRNAs can be as short as about 42-44 nt long of which about 23-25 nt is guide sequence and about 19 nt is the constitutive direct repeat sequence. In contrast, the combined Cas9 tracrRNA and crRNA synthetic sequences can be about 100 nt long.

Second, certain Cpf1 systems prefer a “TTN” PAM motif that is located 5′ upstream of its target. This is in contrast to the “NGG” PAM motifs located on the 3′ of the target DNA for common Cas9 systems such as Streptococcus pyogenes Cas9. In some embodiments, the uracil base immediately preceding the guide sequence cannot be substituted (Zetsche, B. et al. 2015. “Cpf1 Is a Single RNA-Guided Endonuclease of a Class 2 CRISPR-Cas System” Cell 163, 759-771, which is hereby incorporated by reference in its entirety for all purposes).

Third, the cut sites for Cpf1 are staggered by about 3-5 nt, which create “sticky ends” (Kim et al., 2016. “Genome-wide analysis reveals specificities of Cpf1 endonucleases in human cells” published online Jun. 6, 2016). These sticky ends with 3-5 nt overhangs are thought to facilitate NHEJ-mediated-ligation, and improve gene editing of DNA fragments with matching ends. The cut sites are in the 3′ end of the target DNA, distal to the 5′ end where the PAM is. The cut positions usually follow the 18th nt on the non-hybridized strand and the corresponding 23rd nt on the complementary strand hybridized to the crRNA.

Fourth, in Cpf1 complexes, the “seed” region is located within the first 5 nt of the guide sequence. Cpf1 crRNA seed regions are highly sensitive to mutations, and even single base substitutions in this region can drastically reduce cleavage activity (see Zetsche B. et al. 2015 “Cpf1 Is a Single RNA-Guided Endonuclease of a Class 2 CRISPR-Cas System” Cell 163, 759-771). Critically, unlike the Cas9 CRISPR target, the cleavage sites and the seed region of Cpf1 systems do not overlap. Additional guidance on designing Cpf1 crRNA targeting oligos is available on Zetsche B. et al. 2015 (“Cpf1 Is a Single RNA-Guided Endonuclease of a Class 2 CRISPR-Cas System” Cell 163, 759-771).

### D. Guide Nucleic Acids

In some embodiments, a guide nucleic acid (e.g., RNA or DNA) of the present disclosure includes two coding regions, encoding for crRNA and tracrRNA, respectively. In other embodiments, the guide RNA is a single guide RNA (sgRNA) (a synthetic crRNA/tracrRNA hybrid). In other embodiments, the guide RNA is a crRNA for a Cpf1 endonuclease.

Unless otherwise noted, all references to a single guide nucleic acid (e.g., sgRNA or sgDNA) in the present disclosure can be read as referring to a guide nucleic acid (e.g., gRNA or gDNA). Therefore, embodiments described in the present disclosure which refer to a single guide nucleic acid (e.g., sgRNA or sgDNA) will also be understood to refer to a guide nucleic acid (e.g., gRNA or gDNA).

The guide is designed to recruit the CRISPR endonuclease to a target nucleic acid region. Such methods are known in the art. Software programs can be used to identify candidate CRISPR target sequences on both strands of an input DNA sequence based on desired guide sequence length and a CRISPR motif sequence (e.g., PAM) for a specified CRISPR enzyme. For example, target sites for Cpf1 from Francisella novicida U112, with PAM sequences TTN, may be identified by searching for 5′-TTN-3′ both on the input sequence and on the reverse-complement of the input. The target sites for Cpf1 from Lachnospiraceae bacterium and Acidaminococcus sp., with PAM sequences TTTN, may be identified by searching for 5′-TTTN-3′ both on the input sequence and on the reverse complement of the input. Likewise, target sites for Cas9 of S. thermophilus CRISPR, with PAM sequence NNAGAAW, may be identified by searching for 5′-Nx-NNAGAAW-3′ both on the input sequence and on the reverse-complement of the input. The PAM sequence for Cas9 of S. pyogenes is 5′-NGG-3′.

Since multiple occurrences in the genome of the DNA or RNA target site may lead to nonspecific genome editing, after identifying all potential sites, sequences may be filtered out based on the number of times they appear in the relevant reference genome or modular CRISPR construct. For those CRISPR enzymes for which sequence specificity is determined by a “seed” sequence (such as the first 5 nt of the guide sequence for Cpf1-mediated cleavage) the filtering step may also account for any seed sequence limitations.

In some embodiments, algorithmic tools identify potential off target sites for a particular guide sequence. For example, in some embodiments Cas-Offinder can be used to identify potential off target sites for Cpf1 (see Kim et al., 2016. Nature Biotechnology 34, 863-868). Any other publicly available CRISPR design/identification tool may also be used, including for example the Zhang lab crispr.mit.edu tool (see Hsu, et al. 2013 “DNA targeting specificity of RNA guided Cas9 nucleases” Nature Biotech 31, 827-832).

In some embodiments, the user can choose the length of the seed sequence. The user can specify the number of occurrences of the seed: PAM sequence in a genome for purposes of passing the filter. The default is to screen for unique sequences. Filtration level is altered by changing both the length of the seed sequence and the number of occurrences of the sequence in the genome. The program may in addition or alternatively provide the sequence of a guide sequence complementary to the reported target sequence(s) by providing the reverse complement of the identified target sequence(s).

In the guide RNA or DNA, the “spacer/guide sequence” is complementary to the “proto spacer” sequence in the nucleic acid target. The gRNA scaffold for a single stranded gRNA structure (or gDNA scaffold for a single stranded gDNA structure) is recognized by the Cas protein.

In some embodiments, the transgenic plant, plant part, plant cell, or plant tissue culture taught herein includes a recombinant construct, which includes at least one nucleic acid sequence encoding a guide RNA or guide DNA. In some embodiments, the nucleic acid is operably linked to a promoter. In other embodiments, a recombinant construct further comprises a nucleic acid sequence encoding a CRISPR endonuclease. In other embodiments, the guide RNA or DNA is capable of forming a complex with said CRISPR endonuclease, and said complex is capable of binding to and creating a double-strand break in a target nucleic acid sequence of said plant genome. In some embodiments, the CRISPR endonuclease is Cas9. In some embodiments, the CRISPR endonuclease is Cpf1. In some embodiments, the CRISPR endonuclease is Cas13d.

In some embodiments, the modified plant cells include one or more modifications (e.g., insertions, deletions, or mutations of one or more nucleic acids) in the genomic DNA sequence of an endogenous target gene (or RNA of a target sequence). In some embodiments, the modified plant cells include a modified endogenous target gene, such as a mutation, or include additional nucleic acid sequences in an endogenous genome. In some embodiments, the modifications in the genomic DNA sequence cause mutation, thereby altering the function of a target protein. In some embodiments, the modifications in the genomic DNA sequence results in amino acid substitutions, thereby altering or reducing the normal function of the encoded protein. In some embodiments, the modifications in the genomic DNA sequence encode a modified endogenous protein with modulated, altered, stimulated, enhanced, or reduced function compared to the unmodified version of the endogenous protein. In some embodiments, the modifications in the genomic DNA sequence results in additional protein expression.

In some embodiments, modified plants, plant parts, or plant cells include one or more modified endogenous target genes, wherein the one or more modifications result in an enhanced expression of one or more of the target genes, and/or enhanced activity of one or more proteins encoded by the target genes (the target proteins), compared to the expression/activity of a corresponding gene/protein in an unmodified plant, plant part, or plant cell. For example, in some embodiments, a modified plant, plant part, or plant cell demonstrates enhanced expression of a target gene, and/or activity of a target protein. In some embodiments, the expression of the gene or activity of the protein (such as a GELP gene repressor) in a modified plant, plant part, or plant cell is enhanced by at least 0.5%, 1%, 2%, 3%, 4%, 5%, 10%, 20%, 30%, 40%, 50%, 60%, 70%, 80%, 90%, 100%, 200%, 300%, 400% or higher compared to the expression of a corresponding gene/protein in an unmodified plant, plant part, or plant cell.

In some embodiments, the modified endogenous protein demonstrates enhanced binding affinity to another protein expressed by the modified plant cell or by another cell; enhanced signaling capacity; enhanced enzymatic activity; enhanced DNA-binding activity with respect to a specific DNA sequence; or enhanced ability to function as a scaffolding protein.

In some embodiments, modified plants, plant parts, or plant cells comprise one or more modified endogenous target genes, wherein the one or more modifications results in increased or reduced expression of one or more of the target genes, and/or increased or reduced activity of one or more proteins encoded by the target genes (the target proteins), compared to the expression/activity of a corresponding gene/protein in an unmodified plant, plant part, plant cell (e.g., a “unmodified endogenous protein”). For example, in some embodiments, a modified plant, plant part, or plant cell demonstrates reduced expression of a target gene, and/or activity of a target protein. In some embodiments, the expression of the gene or activity of the protein in a modified plant, plant part, or plant cell is increase or reduced by at least 0.5%, 1%, 2%, 3%, 4%, 5%, 10%, 20%, 30%, 40%, 50%, 60%, 70%, 80%, 90%, or 100% compared to the expression of a corresponding gene/protein in an unmodified plant, plant part, or plant cell.

### Tissue Cultures and In Vitro Regeneration of Basil Plants

Tissue cultures of one or more of the ‘OCB-029’, ‘OCB-017’, ‘OCB-022’, ‘OCB-009’, ‘OCB-011’, ‘OCB-027’, ‘OCB-019’, ‘OCB-031’, ‘OCB-121’, ‘OCB-174’, ‘OCB-169’, ‘OCB-153’, ‘OCB-115’, ‘OCB-120’, ‘OCB-119’, ‘OCB-067’, ‘OCB-058’, ‘OCB-086’, ‘OCB-062’, ‘OCB-117’, ‘OCB-057’, ‘OCB-126’, ‘OCB-188’, ‘OCB-129’, or ‘OCB-051’ basil variety are provided. A tissue culture includes isolated cells of the same or a different type or a collection of such cells organized into parts of a plant. Exemplary types of tissue cultures include protoplasts, calli and plant cells that are intact in plants or parts of plants, such as embryos, pollen, flowers, leaves, roots, root tips, anthers, meristematic cells, pistil, seed, petiole, stein, ovule, cotyledon, hypocotyl, shoot or stem, and the like. In a particular example, the tissue culture includes embryos, protoplasts, meristematic cells, pollen, leaves or anthers of the ‘OCB-029’, ‘OCB-017’, ‘OCB-022’, ‘OCB-009’, ‘OCB-011’, ‘OCB-027’, ‘OCB-019’, ‘OCB-031’, ‘OCB-121’, ‘OCB-174’, ‘OCB-169’, ‘OCB-153’, ‘OCB-115’, ‘OCB-120’, ‘OCB-119’, ‘OCB-067’, ‘OCB-058’, ‘OCB-086’, ‘OCB-062’, ‘OCB-117’, ‘OCB-057’, ‘OCB-126’, ‘OCB-188’, ‘OCB-129’, and/or ‘OCB-051’ basil variety disclosed herein. Also provided are basil plants regenerated from such tissue cultures, wherein the regenerated basil plant expresses the physiological and morphological characteristics of a new basil variety disclosed herein.

Methods for preparing tissue cultures of regenerable basil cells and regenerating basil plants therefrom, are known, such as those disclosed in U.S. Pat. Nos. 4,992,375; 5,015,580; 5,024,944, and 5,416,011. Tissue culture provides the capability to regenerate fertile plants. This can allow, for example, transformation of the tissue culture cells followed by regeneration of transgenic plants. For transformation to be efficient and successful, DNA can be introduced into cells that give rise to plants or germ-line tissue.

Basil plants can be regenerated using shoot morphogenesis or somatic embryogenesis. Shoot morphogenesis is the process of shoot meristem organization and development. Shoots grow out from a source tissue and are excised and rooted to obtain an intact plant. During somatic embryogenesis, an embryo (similar to the zygotic embryo), containing both shoot and root axes, is formed from somatic plant tissue. An intact plant rather than a rooted shoot results from the germination of the somatic embryo.

Shoot morphogenesis and somatic embryogenesis are different processes and the specific route of regeneration is primarily dependent on the explant source and media used for tissue culture manipulations. While the systems are different, both systems show variety-specific responses where some lines are more responsive to tissue culture manipulations than others. A line that is highly responsive in shoot morphogenesis may not generate many somatic embryos, while lines that produce large numbers of embryos during an “induction” step may not give rise to rapidly-growing proliferative cultures. In addition to line-specific responses, proliferative cultures can be observed with both shoot morphogenesis and somatic embryogenesis. Proliferation allows a single, transformed cell to multiply to the point that it can contribute to germ-line tissue.

Shoot morphogenesis is a system whereby shoots are obtained de novo from cotyledonary nodes of basil seedlings (Wright et al., 1986. Plant Cell Reports 5:150-154). The shoot meristems form subepidermally and morphogenic tissue can proliferate on a medium containing benzyl adenine (BA). This system can be used for transformation if the subepidermal, multicellular origin of the shoots is recognized and proliferative cultures are utilized. Tissue that can give rise to new shoots are targeted and proliferated within the meristematic tissue to lessen problems associated with chimerism.

Somatic embryogenesis in basil is a system in which embryogenic tissue is obtained from the zygotic embryo axis (Christianson et al., 1983. Science 222:632-634). The embryogenic cultures are proliferative and the proliferative embryos are of apical or surface origin with a small number of cells contributing to embryo formation. The origin of primary embryos (the first embryos derived from the initial explant) is dependent on the explant tissue and the auxin levels in the induction medium (Hartweck et al., 1988. In Vitro Cell. Develop. Bio. 24:821-828). With proliferative embryonic cultures, single cells or small groups of surface cells of the “older” somatic embryos form the “newer” embryos.

Embryogenic cultures can also be used for regeneration, including regeneration of transgenic plants.

### Methods of Making Extracts from Basils

Extracts can be generated from the ‘OCB-029’, ‘OCB-017’, ‘OCB-022’, ‘OCB-009’, ‘OCB-011’, ‘OCB-027’, ‘OCB-019’, ‘OCB-031’, ‘OCB-121’, ‘OCB-174’, ‘OCB-169’, ‘OCB-153’, ‘OCB-115’, ‘OCB-120’, ‘OCB-119’, ‘OCB-067’, ‘OCB-058’, ‘OCB-086’, ‘OCB-062’, ‘OCB-117’, ‘OCB-057’, ‘OCB-126’, ‘OCB-188’, ‘OCB-129’, and/or ‘OCB-051’ basil variety or progeny thereof. Such extracts can be used in aroma and flavoring and medicinal or health oriented plant based extracts. In some examples, the extract includes genetic material from the ‘OCB-029’, ‘OCB-017’, ‘OCB-022’, ‘OCB-009’, ‘OCB-011’, ‘OCB-027’, ‘OCB-019’, ‘OCB-031’, ‘OCB-121’, ‘OCB-174’, ‘OCB-169’, ‘OCB-153’, ‘OCB-115’, ‘OCB-120’, ‘OCB-119’, ‘OCB-067’, ‘OCB-058’, ‘OCB-086’, ‘OCB-062’, ‘OCB-117’, ‘OCB-057’, ‘OCB-126’, ‘OCB-188’, ‘OCB-129’, and/or ‘OCB-051’ basil variety, such as a cell from the ‘OCB-029’, ‘OCB-017’, ‘OCB-022’, ‘OCB-009’, ‘OCB-011’, ‘OCB-027’, ‘OCB-019’, ‘OCB-031’, ‘OCB-121’, ‘OCB-174’, ‘OCB-169’, ‘OCB-153’, ‘OCB-115’, ‘OCB-120’, ‘OCB-119’, ‘OCB-067’, ‘OCB-058’, ‘OCB-086’, ‘OCB-062’, ‘OCB-117’, ‘OCB-057’, ‘OCB-126’, ‘OCB-188’, ‘OCB-129’, and/or ‘OCB-051’ basil variety.

In one example, plants of the ‘OCB-029’, ‘OCB-017’, ‘OCB-022’, ‘OCB-009’, ‘OCB-011’, ‘OCB-027’, ‘OCB-019’, ‘OCB-031’, ‘OCB-121’, ‘OCB-174’, ‘OCB-169’, ‘OCB-153’, ‘OCB-115’, ‘OCB-120’, ‘OCB-119’, ‘OCB-067’, ‘OCB-058’, ‘OCB-086’, ‘OCB-062’, ‘OCB-117’, ‘OCB-057’, ‘OCB-126’, ‘OCB-188’, ‘OCB-129’, and ‘OCB-051’ basil variety, or any above-ground part of the plant, are harvested, for example, after at least 20 days, at least 30 days, at least 45 days, at least 60 days, at least 70 days, at least 90 days, at least 100 days, or at least 120 days of growth (such as after 45 to 100 days, 60 to 100 days, or 50 to 90 days, such as after 60 days or 90 days of growth). The plant can be dried, for example by leaving it in the field to partially dry, or brought indoors to be flash frozen, frozen, boiled, heated, or dried, for example at 37° C. (e.g., by air drying, microwaving, lyophilization, or combinations thereof, such as by using a Powell walk-in forced air dryer) or other drying system until no further moisture loss is noted under the temperature and pressure and relative humidity of the drying system. The leaves and flowers of the plant can be separated from the stems, for example manually or by machine. Essential oils can be extracted from the dried leaves and flowers using steam or hydro-distillation or hot water. In some examples, solvent extraction and super critical fluids are used.

The sweet basil from which an extract is generated can be field-grown, greenhouse grown or grown in pots, sacs and containers, and cut at any height above the soil, and the plant distilled fresh or partially dried to obtain the aromatic essential oil. Sweet basil plants are typically cut once per growing season, but the ‘OCB-029’, ‘OCB-017’, ‘OCB-022’, ‘OCB-009’, ‘OCB-011’, ‘OCB-027’, ‘OCB-019’, ‘OCB-031’, ‘OCB-121’, ‘OCB-174’, ‘OCB-169’, ‘OCB-153’, ‘OCB-115’, ‘OCB-120’, ‘OCB-119’, ‘OCB-067’, ‘OCB-058’, ‘OCB-086’, ‘OCB-062’, ‘OCB-117’, ‘OCB-057’, ‘OCB-126’, ‘OCB-188’, ‘OCB-129’, and ‘OCB-051’ basil varieties can be harvested (or cut) once or twice or more per growing season, provided it is grown with ample water, nutrients and under environmental conditions that result in plant growth and development. Once harvested, the plant can be distilled immediately, allowed to be partially dried in full sun, partial sun in the field and then placed into a container for steam or hydro-distillation or allowed to further dried and processed at future time. Other processes can also be used, including but not limited to, solvent extraction. For an extract or dry product, the sweet basil may be sun dried, dried in shade, with or without artificial heat introduced by different sources, and then allowed to dry before extraction.

### Products Containing Basil

The disclosure provides products obtained from one or more of the new varieties (‘OCB-029’, ‘OCB-017’, ‘OCB-022’, ‘OCB-009’, ‘OCB-011’, ‘OCB-027’, ‘OCB-019’, ‘OCB-031’, ‘OCB-121’, ‘OCB-174’, ‘OCB-169’, ‘OCB-153’, ‘OCB-115’, ‘OCB-120’, ‘OCB-119’, ‘OCB-067’, ‘OCB-058’, ‘OCB-086’, ‘OCB-062’, ‘OCB-117’, ‘OCB-057’, ‘OCB-126’, ‘OCB-188’, ‘OCB-129’, and ‘OCB-051’) disclosed herein, or progeny thereof. Exemplary products include a biomass or part thereof, such as an extract, oil, protein isolate, protein concentrate, oil extract, or leaves. For example a dried biomass and/or leaves of one or more of the new varieties (‘OCB-029’, ‘OCB-017’, ‘OCB-022’, ‘OCB-009’, ‘OCB-011’, ‘OCB-027’, ‘OCB-019’, ‘OCB-031’, ‘OCB-121’, ‘OCB-174’, ‘OCB-169’, ‘OCB-153’, ‘OCB-115’, ‘OCB-120’, ‘OCB-119’, ‘OCB-067’, ‘OCB-058’, ‘OCB-086’, ‘OCB-062’, ‘OCB-117’, ‘OCB-057’, ‘OCB-126’, ‘OCB-188’, ‘OCB-129’, and ‘OCB-051’) or progeny thereof can be used as part of food, beverage, or aroma-based product. In some examples, the product includes at least one cell, DNA, and/or protein of basil variety ‘OCB-029’, ‘OCB-017’, ‘OCB-022’, ‘OCB-009’, ‘OCB-011’, ‘OCB-027’, ‘OCB-019’, ‘OCB-031’, ‘OCB-121’, ‘OCB-174’, ‘OCB-169’, ‘OCB-153’, ‘OCB-115’, ‘OCB-120’, ‘OCB-119’, ‘OCB-067’, ‘OCB-058’, ‘OCB-086’, ‘OCB-062’, ‘OCB-117’, ‘OCB-057’, ‘OCB-126’, ‘OCB-188’, ‘OCB-129’, and/or ‘OCB-051’.

The disclosure provides nanoparticles which includes parts (such as leaves or extract(s)) of one or more of the new varieties (‘OCB-029’, ‘OCB-017’, ‘OCB-022’, ‘OCB-009’, ‘OCB-011’, ‘OCB-027’, ‘OCB-019’, ‘OCB-031’, ‘OCB-121’, ‘OCB-174’, ‘OCB-169’, ‘OCB-153’, ‘OCB-115’, ‘OCB-120’, ‘OCB-119’, ‘OCB-067’, ‘OCB-058’, ‘OCB-086’, ‘OCB-062’, ‘OCB-117’, ‘OCB-057’, ‘OCB-126’, ‘OCB-188’, ‘OCB-129’, and/or ‘OCB-051’). In some examples, the parts (such as leaves or extract(s)) of one or more of the new varieties are encapsulated in a nanoparticle.

The disclosure provides containers, such as a glass, paper, or plastic container, which includes leaves of one or more of the new varieties (‘OCB-029’, ‘OCB-017’, ‘OCB-022’, ‘OCB-009’, ‘OCB-011’, ‘OCB-027’, ‘OCB-019’, ‘OCB-031’, ‘OCB-121’, ‘OCB-174’, ‘OCB-169’, ‘OCB-153’, ‘OCB-115’, ‘OCB-120’, ‘OCB-119’, ‘OCB-067’, ‘OCB-058’, ‘OCB-086’, ‘OCB-062’, ‘OCB-117’, ‘OCB-057’, ‘OCB-126’, ‘OCB-188’, ‘OCB-129’, and/or ‘OCB-051’). The leaves can be dried, frozen, or fresh. In some examples, the container includes leaves (or other parts) from other plants, such as oregano, parsley, marjoram, thyme, rosemary, or non-sweet basils, or combinations thereof. In some examples, the container includes garlic, such as dried or fresh garlic.

Provided herein is a dried tea, food and flavor or fragrance product which includes leaves or comes from an oil extract, and/or biomass of one or more of the new varieties (‘OCB-029’, ‘OCB-017’, ‘OCB-022’, ‘OCB-009’, ‘OCB-011’, ‘OCB-027’, ‘OCB-019’, ‘OCB-031’, ‘OCB-121’, ‘OCB-174’, ‘OCB-169’, ‘OCB-153’, ‘OCB-115’, ‘OCB-120’, ‘OCB-119’, ‘OCB-067’, ‘OCB-058’, ‘OCB-086’, ‘OCB-062’, ‘OCB-117’, ‘OCB-057’, ‘OCB-126’, ‘OCB-188’, ‘OCB-129’, and/or ‘OCB-051’ or progeny thereof. Also provided is a liquid tea, produced from leaves, oil extract, and/or biomass of one or more of the new varieties (‘OCB-029’, ‘OCB-017’, ‘OCB-022’, ‘OCB-009’, ‘OCB-011’, ‘OCB-027’, ‘OCB-019’, ‘OCB-031’, ‘OCB-121’, ‘OCB-174’, ‘OCB-169’, ‘OCB-153’, ‘OCB-115’, ‘OCB-120’, ‘OCB-119’, ‘OCB-067’, ‘OCB-058’, ‘OCB-086’, ‘OCB-062’, ‘OCB-117’, ‘OCB-057’, ‘OCB-126’, ‘OCB-188’, ‘OCB-129’, and/or ‘OCB-051’) or progeny thereof.

Provided herein are pet toys or aromatic toys/balls/ornamentals/aromatic wreaths/other personal consumer items, which include leaves, oil extract, and/or biomass of one or more of the new varieties (‘OCB-029’, ‘OCB-017’, ‘OCB-022’, ‘OCB-009’, ‘OCB-011’, ‘OCB-027’, ‘OCB-019’, ‘OCB-031’, ‘OCB-121’, ‘OCB-174’, ‘OCB-169’, ‘OCB-153’, ‘OCB-115’, ‘OCB-120’, ‘OCB-119’, ‘OCB-067’, ‘OCB-058’, ‘OCB-086’, ‘OCB-062’, ‘OCB-117’, ‘OCB-057’, ‘OCB-126’, ‘OCB-188’, ‘OCB-129’, and/or ‘OCB-051’) or progeny thereof.

Oil extracts of one or more of the new varieties (‘OCB-029’, ‘OCB-017’, ‘OCB-022’, ‘OCB-009’, ‘OCB-011’, ‘OCB-027’, ‘OCB-019’, ‘OCB-031’, ‘OCB-121’, ‘OCB-174’, ‘OCB-169’, ‘OCB-153’, ‘OCB-115’, ‘OCB-120’, ‘OCB-119’, ‘OCB-067’, ‘OCB-058’, ‘OCB-086’, ‘OCB-062’, ‘OCB-117’, ‘OCB-057’, ‘OCB-126’, ‘OCB-188’, ‘OCB-129’, and/or ‘OCB-051’) or progeny thereof are provided, and in one example are formulated into a spray.

### Example 1

### Materials and Methods

This example provides materials and methods used to generate the data described in the subsequent Examples. Additional details may be found in Brindisi et al., Genetic linkage mapping and quantitative trait locus (QTL) analysis of sweet basil (Ocimum basilicum L.) to identify genomic regions associated with cold tolerance and major volatiles, PLOS One, 19(4):e0299825, Apr. 9, 2024, herein incorporated by reference in it is entirely including all supplementary materials, and Brindisi, Breeding sweet basil (Ocimum basilicum L.) for chilling tolerance using advance genetic and phytochemical techniques, dissertation, Rutgers, The State University of New Jersey, May 2023, 194 pages (doi.org/doi:10.7282/t3-10s3-xg76) herein incorporated by reference in its entirety.

### Plant Material

Two inbred basil lines (Ocimum basilicum L.) were selected as parents: 1) the commercial variety ‘Rutgers Obsession DMR’ (9) and 2) a breeding line developed for cold tolerance known as ‘CB15’ (a). ‘Rutgers Obsession DMR’ was selected for its desirable sweet basil aroma profile, downy mildew tolerance, Fusarium oxysporum f. sp. basilica (FOB) resistance, moderate leaf size and late flowering time, but is sensitive to cold injury akin to other commercial basil varieties. ‘CB15’ was selected for its cold tolerance, but is rich in estragole and lacks a sweet basil aroma, has large leaves and no BDM resistance or tolerance or FOB resistance and flowers early. The parents were crossed to obtain an individual F1 hybrid, which was then selfed to obtain 200 F₂ progeny as the Obsession x Chilling Basil (OCB) mapping population. F₂ seeds were sown in 128-cell trays with standard potting soil on May 3, 2021 and transplanted into 3.5″ square pots after 6 weeks. Plants were transplanted into round 2 gallon pots 9 weeks after sowing and pruned to maintain the vegetative stage. The parents, hybrid and progeny were maintained as mature plants in the Rutgers New Jersey Agricultural Experiment Station (NJAES) greenhouses in New Brunswick, NJ. Plants were grown in natural light supplemented with 400 W, 208V high pressure sodium (HPS) HID lamps (P. L. Light Systems, Beamsville, ON, Canada) on a 18 h/6 h light/dark photoperiod with −27° C./20° C. day/night cycle at 70% relative humidity. Plants were watered daily and fertilized with 20-20-20 biweekly from April to September and 20-10-20 from October to March. An IPM program was implemented with beneficial insects and occasional pesticide applications for pest control.

### Chilling Assay

Leaves were assessed for necrosis based on the postharvest chilling methods of Brindisi et al. [43]. Fresh, healthy leaves were harvested from each mature plant and sealed in a perforated plastic bag. Bags (n=3) containing 3-5 leaves were collected for each parent, F₂ plant and the F1 hybrid. One bag from each plant was randomly placed in a cardboard box in a completely randomized block design (CRBD). The three cardboard boxes were treated as blocks and randomly arranged in a walk-in refrigerator (Mr. Winter, 1992, 593.7 ft³) in the NJAES greenhouse. The leaves were refrigerated for 4 days at 3-5° C. The entire CRBD was repeated on three dates: Oct. 7, 2021, Dec. 2, 2021 and Feb. 2, 2022. Basil leaves in each bag were photographed together in photo studio light boxes (Glendan, 12″×12″) against a black background. Images were acquired using mobile devices (iPhone 7-12). The percent leaf necrosis was evaluated by the machine learning program, Leaf Necrosis Classifier (LNC) [48, 49] by eliminating the background and comparing the number of pixels of necrotic or brown leaf tissue to the number of pixels of the entire leaf blade. The final percent leaf necrosis value for each of the plants was determined by averaging the blocks in each replicate. A low percent necrosis (0%) represents chilling tolerance while a high percent necrosis (100%) represents chilling sensitivity.

### Aroma Volatile Analysis

Dried and ground leaf samples from the F₂ plants, parents and F1 were analyzed in triplicate (n=3) using a Shimadzu Gas Chromatograph (GC) 2010 Plus Series via Headspace Solid Phase Microextraction (SPME) with the Shimadzu TQ8040 Triple-Q Mass Spectrometer (MS) with AOC 6000 autosampler. Injections were extracted for 5 min at 250° C. and desorbed for 2 min with an analysis time of 19 min. Samples were subject to a 5 split with a sampling time of 1 min. Helium was used as the carrier gas at a pressure of 47.7 kPA with a total flow of 2 mL/min and a column flow of 1 mL/min. The column was a SH-Rxi-5Sil MS fused silica capillary column of 0.25 um thickness, 30 m length and 0.25 mm diameter. The column oven temperature was set to 35° C. for 4 min and then to 250° C. at a rate of 20° C./min and held for 2 min. The ion source temperature was 200° C. and the interface temperature was 250° C. Mass spectra were observed from 4 min to 16.75 min in the Q1 scan acquisition mode. Scan speed was 10000 from 45 m/z to 500 m/z. Aroma volatiles compounds were identified by comparison with their retention indices relative to the multiple n-alkanes and by the mass spectra fragmentation pattern of each component compared to published mass spectra [50]. Three major compounds were quantified for QTL analysis: estragole, linalool and eucalyptol. The concentration of each compound was calculated as peak area. The final peak area value for each of the plants was determined by averaging the replicates by compound in absorbance units (A) and dividing by the mass (g) of the plant material.

### Statistics

The Shapiro-Wilk Normality Test was used to test normality of phenotypic data in R/stats v4.2.1 and skewness was assessed using R/moments v0.14.1 [51]. Kruskal-Wallis Rank Sum Test one-way analysis of variance was used as a non-parametric assessment of one-way analysis of variance to assess the global differences in phenotypic traits at a 5% level of significance (p-value <0.05) among F₂ lines for each year, and Spearman's Rank correlation coefficient was used to determine the strength of the linear relationship between pairs of phenotypes in R/stats v4.2.1. Phenotype data were transformed for QTL identification using Tukey's Ladder of Powers in R/rcompanion v2.4.16 [51, 52].

### Flow Cytometric Analysis

Nuclear DNA content was determined with flow cytometry for the parents, hybrid and three randomly selected progeny (OCB-010, OCB-065 and OCB-180) by Heavenly Gardens (Galloway, OH, USA). Nuclei were extracted from fresh leaf tissue and nuclear DNA was stained with the CyStain® PI Absolute P reagent kit. Samples were analyzed on a BD Accuri™ C6 Plus Personal Flow Cytometer (BD Biosciences, Franklin Lakes, NJ, USA). Laser excitation was set to 488 nm and 640 nm. Emissions were detected through 4 colors with standard optical filters: FL1 533/30 nm, FL2 585/40 nm, FL3>670 nm and FL4 675/25 mm. Tomato (Solanum lycopersicum) and Daylily ‘Purple Pixie Gumdrop’ (Hemerocallis) with estimated genome sizes (2 C DNA content) of 2.05 Gb and 8.59 Gb, respectively, were included as internal controls for each sample.

DNA extraction

Young leaf tissue was frozen in liquid nitrogen and ground with a bead mill (TissueLyser II; Qiagen, Hilden, Germany) using tungsten carbide beads and adapter sets pre-cooled at −80° C. Genomic DNA (gDNA) was extracted using the E.Z.N.A.® SP Plant DNA Kit (Omega BioTek, Norcross, GA) and assessed for quantity and quality using 260/280 absorbance ratios on a spectrophotometer (Nanodrop, Thermo Fisher Scientific, Waltham, MA). Samples of high quality (A260/280 ratio between 1.8 and 2.0) were diluted to 50 ng/L for DNA sequencing library preparation.

### Library Preparation

Genotyping-by-sequencing (GBS) double digest restriction-site associated DNA sequencing (ddRADseq) libraries were prepared with the enzymes PstI (NEB, USA) and MspI (NEB, USA) according to the methods of Pyne et al. [16], which was modified from the original approach of Poland et al. [53]. One library was prepared for each progeny and at least ten libraries were prepared for each parent to generate a 10× parental sequencing depth. Libraries were quantified using a Qubit fluorometer (Thermo Fisher Scientific, Waltham, MA). Sample libraries were normalized to 6-7 ng/uL and pooled into 5 sets with 48 individual libraries per set. Each pooled library set was sequenced in its own lane of a Hi-Seq 2000 (Illumina, USA) using 2×150 bp, paired-end sequencing by Genewiz (Azenta Life Sciences, South Plainfield, New Jersey).

SNP calling

The Stacks v2.60 [54] software was used to convert raw paired-end reads into genotype data with BWA v0.7.17 [55] alignment and SAMtools v1.8 [56] file processing. Sets were filtered for quality, trimmed to 100 bp and demultiplexed with the Stacks process_radtags program. Reads with an uncalled base or low-quality scores were removed. Replicate parent radtag files were concatenated. The Ocimum basilicum reference genome [20] was indexed with the BWA index command to which progeny and parent radtags were aligned with the BWA mem command. Shorter split hits were marked as secondary. Resulting SAM files were converted to BAM files and sorted with the SAMtools view and sort commands. Loci were built using the Stacks ref_map.pl using program default settings. The Stacks populations program was used to export loci as an F2 mapping population in the R/QTL format with a minimum percentage of progeny individuals required to process a locus set to 90%.

### Genetic Map Construction

Linkage mapping was performed using R/QTL v1.52 [57] in R/RStudio v4.2.1 [51, 58]. Null, duplicate and potentially switched markers were removed. The general likelihood ratio was used to estimate the genetic map with the markerlrt command instead of recombination fractions with the est.rf command due to extensive segregation distortion. Segregation distortion was determined with a Bonferroni correction (p-value <0.01). Markers aligning to LG99 were moved to linked groups or dropped if there was no linkage. Problematic markers were identified with the droponemarker command and markers with no linkage were eliminated from the analysis. One individual was removed as an outlier due to its high number of crossover events. The genotyping error rate was calculated with a log 10 likelihood estimate and used to identify and subsequently remove genotyping errors.

### QTL Analysis

Quantitative trait locus (QTL) mapping was conducted in R/QTL. The hidden Markov model calculated the probabilities underlying genotypes with the calc.genoprob command at a maximum distance of 1 cM and a genotyping error rate of 0.0125. Significance thresholds (α=0.05) were determined for the single QTL model using the scanone command with 1000 permutations for each replicate. A genome scan with a single QTL model was performed using the Haley-Knott regression mapping method and 1000 permutations. Confidence intervals were determined with 95% bayes credible intervals. QTL effect size was estimated from linear regression using the effects can command. A genome scan with a two-QTL model was also performed using the Haley-Knot method and 1000 permutations. A maximum distance of 2.5 cM was used to calculate the probabilities underlying genotypes and a genotyping error rate of 0.0125. Significance thresholds (α=0.05) were determined for the 2D model using scan two with 1000 permutations for each replicate. MQM was implemented using the fitqtl command based on the significant results of the single and 2D analyses. The commands addint, addqtl and addpair were used to search for interactions and QTL outside of the model. The model was further tested with forward/backward selection via penalized LOD scores using stepwiseqtl.

### Gene Annotation

The MAKER pipeline was used to create a draft annotation of the O. basilicum ‘RUSB22’ sweet basil genome. Parts of the annotation were completed on the Jetstream Cloud Atmosphere courtesy of XSEDE [59-61]. A repeat library specific to sweet basil was generated using RepeatMasker [62] and MITE-Hunter [63]. The ‘RUSB22’ trinity assembly from Allen et al. [64] and the proteome from Arabidopsis thaliana and Thymus vulgaris [65] along with the repeat library served as the input fasta files for the maker opts control file. Initial MAKER annotations were generated based on the aligned transcript and protein sequence alignment. MAKER gene models with AED of 0.25 or better and greater than 50 amino acids in length were selected for SNAP training [66]. This first round of ab initio gene prediction by SNAP was used to improve upon the next round of MAKER annotation and each round of ab initio gene prediction was used to train subsequent rounds. MAKER annotation was run iteratively for three rounds of SNAP training. The predicted transcripts were provided as input into the software package GOFeat [67, 68]. GOFeat aligns transcript sequences to several databases including NCBI, UniProt, InterPro, KEGG, Pfam and SEED.

Flanking sequences for SNPs generated from the OCB mapping population that were associated with chilling tolerance and aroma QTLs were aligned to the annotated ‘RUSB22’ genome using BLAST and the Burrow-Wheeler Aligner [69]. Annotations within the QTLs for chilling response and aroma compounds were extracted. Annotations in the QTLs associated with cold response were searched for key words including cold, stress, temperature, chill, freeze and freezing and specific gene or gene families. Annotations in the QTLs associated with aroma were searched for key words, including aroma, smell, taste, flavor, sensor and volatile and specific gene or gene families.

### Example 2

### Chilling Tolerance of New Basils

The chilling response of the two parents, F1 hybrid and 200 F₂ individuals were assessed in October 2021, December 2021 and February 2022. In all evaluations, the parents represented the extreme phenotypic classes (FIG. 1). In all evaluations, the chilling tolerant parent ‘CB15’ exhibited <5% necrosis. In October and February, the chilling sensitive parent ‘RU Obsession DMR’ exhibited >50% necrosis. In December, ‘Rutgers Obsession DMR’ exhibited only ˜9% necrosis, however the average chilling response was lower in this replicate than the others and the ‘Rutgers Obsession DMR’ parent still represented the extreme sensitive phenotype. The average percent necrosis of the entire population was 21.3% in October, 6.8% in December and 18.2% in February (FIG. 2).

After comparing each of these three assessments, 19% of the F₂ individuals displayed a “chilling tolerant” response similar to ‘CB15’ (<5% necrosis), 12% displayed a “chilling sensitive” response similar to the ‘Rutgers Obsession DMR’ parent (>30% necrosis) and 69% displayed a “median response” similar to the F1 (5-30% necrosis) as shown in FIG. 3. The phenotype segregated quantitatively in the mapping population with a continuous variation of chilling response (FIG. 4A-4C). The frequency of distributions was non-normal (p-value <0.05) and skewed towards tolerance (0.98-2.4), thus the data were transformed for analysis.

### Example 3

### Aroma Volatile Analysis

The aroma profiles of the two parents, F1 hybrid and 200 F₂ individuals were analyzed for each plant in triplicate. Three major aromatic compounds were detected in the parent with the potential for segregation: estragole (C₁₀H₁₂O), eucalyptol (C₁₀H₁₈O), and linalool (C₁₀H₁₈O). The volatile profile of ‘CB15’ exhibited high estragole (3.48E+08 A/g), low eucalyptol (1.74E+08 A/g) and low linalool (1.72E+08 A/g) as shown in FIG. 5A. In contrast, the volatile profile of ‘Rutgers Obsession DMR’ exhibited low estragole (0 A/g), high eucalyptol (2.82E+08 A/g) and high linalool (3.09E+08 A/g). High and low volatile content is relative to accessions in this population and do not necessarily compare to other basils.

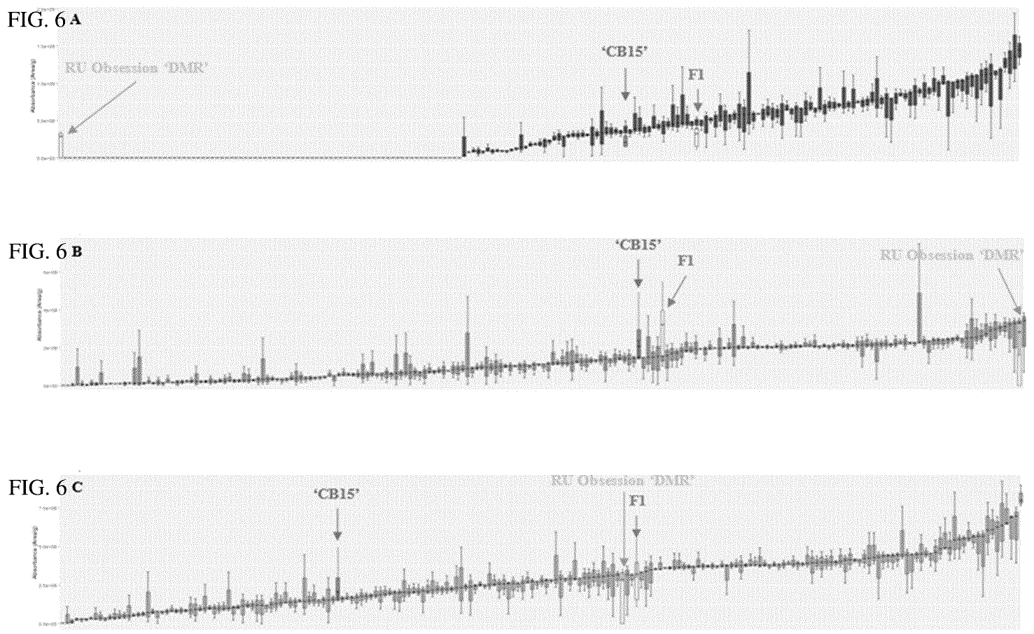

The phenotypes of the aroma compounds segregated quantitatively in the mapping population with a continuous variation for the aroma compounds. (FIGS. 6A-6C). Estragole had the highest mean peak area, followed by linalool and eucalyptol in the F₂ population (FIG. 5B). Despite having the highest mean peak area, a large portion of the F₂ population (41.5%) had no detectable levels of estragole and the rest had moderate to high levels of estragole (7.6e+07-1.44e+09 A). All F₂ individuals had moderate to high levels of linalool (1.3e+07-8.2e+08 A) and low to high levels of eucalyptol (2.3e+05-4.0e+08 A). Transgressive segregation was also observed with 42% of F₂ individuals having higher estragole levels than the ‘CB15’ parent, 61% having lower levels of eucalyptol content than the ‘CB15’ parent, 30% having lower linalool levels than the ‘CB15’ parent and 42% having higher linalool levels than the ‘Rutgers Obsession DMR’ parent. The frequency of distributions was non-normal (p-value <0.05), thus the data were transformed for analysis.

### Example 4

### Phenotypic Correlations

Chilling assays were moderately correlated to each other (p=0.29-0.47; FIG. 7). There was no correlation between the results of the chilling assays and the aroma analyses (p=−0.03-0.14). Aroma compounds were moderately to strongly correlated with each other. Eucalyptol was moderately correlated to linalool (p=0.48) and estragole (p=0.54). Linalool was highly correlated to estragole (p=0.84).

### Example 5

### Flow Cytometric Analysis

The total estimated genome size of all analyzed accession was 4.85-4.88 Gb (2 C DNA content). The similarity in DNA content strongly suggests that the parents, resulting hybrid and F₂ progeny each have the same number of chromosomes. This genome size is consistent with previous literature for tetraploid basil [9, 10].

### Example 6

### Genotyping and Genetic Mapping

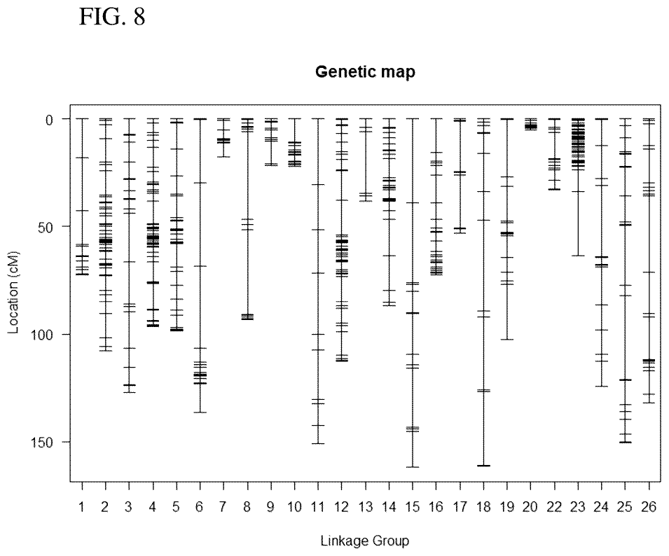

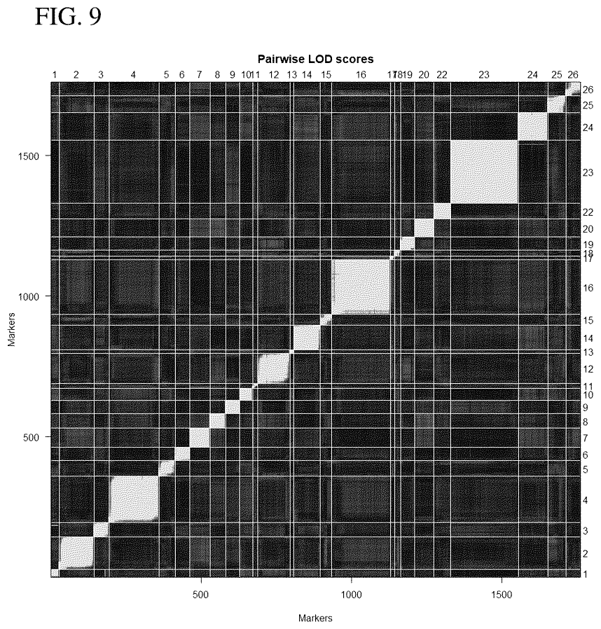

The 200 F₂ progeny and parents were genotyped using ddRADSeq. Sequencing yielded 1.98 billion raw reads and 593,628 Mbases. The mean quality score was 35.73. Reads retained from the parents were 11-27× that of the F₂ individuals. The Stacks software pipeline was used to generate 67,575 loci with 3,558 mappable markers. Removal of null, duplicate, potentially switched and unlinked markers and problematic individuals resulted in a genetic map of 1,761 polymorphic SNP markers ordering into 25 linkage groups (FIG. 8). The original reference map has 26 linkage groups, however LG21 is absent in this analysis due to an insufficient number of quality markers. The total distance on the genetic map was 2,241.8 cM, with an average linkage group length of 90 cM. The shortest LG was 5 cM, while the longest LG was 162 cM. The average number of markers per LG was 70.4, varying from 13 to 225. Segregation of genotypes was distorted with a ratio of 28.4% AA:24.5% AB:47.1% BB (˜1:1:2) instead of 1 AA:2 AB:1 BB, with 98.5% of markers considered to be highly distorted (p-value <0.01). The heatmap of the marker-pairwise estimated recombination fractions vs LOD scores supported that there is only linkage within each LG (FIG. 9).

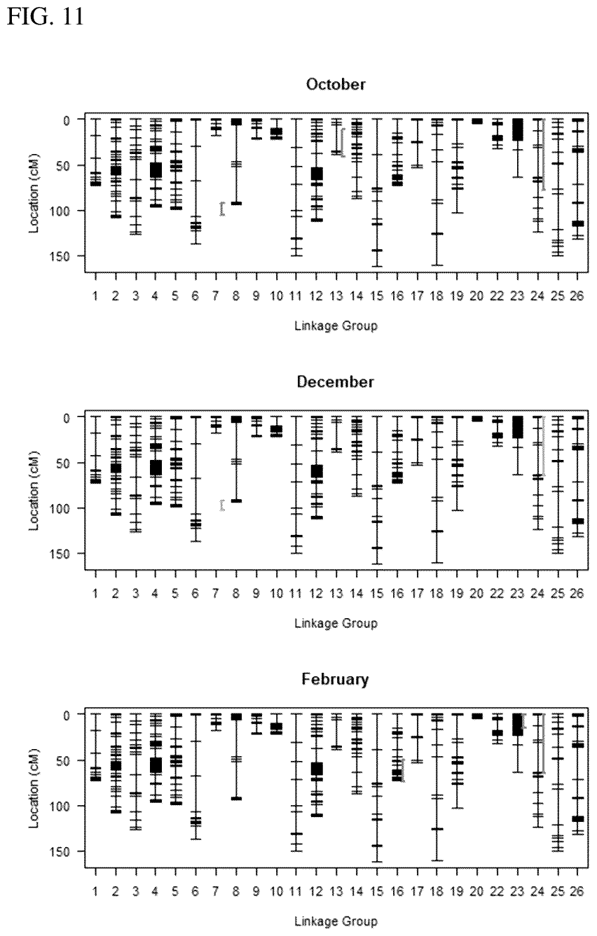

Five significant QTL were detected for chilling response with one significant QTL consistent across all three replications in the single-QTL model (FIGS. 10-11; Table 2). The LOD thresholds (α=0.05) were determined to be 3.70, 3.96 and 3.81 for the chilling assays conducted in October, December and February, respectively. In all replicates, the QTL started at 0.3 cM and spanned to 78 cM in the October replicate or 65 cM in the other two replicates. The QTL on LG24 (qCH24) was significant across all three replicates (LOD=12.8, 10.4 and 4.1) and had the largest effect in the October and December replicates (PVE=25.6% and 21.4%), with the second largest effect in the February replicate (PVE=9.0%). In all replicates, the confidence interval for qCH24 started at 0.3 cM and spanned to ˜70 cM. The QTL on LG16 (qCH16) was only significant in the February replicate (LOD=4.3) and had a slightly higher effect (PVE=9.5%) than the QTL on LG24. The QTL on LG7 (qCH7) was significant in the October and December replicates (LOD=4.7, 6.1; PVE=10.3% and 13.2%). The QTL on LG13 (qCH13) was significant in the October replicate only (LOD=4.5; PVE=9.8%), and the QTL on LG23 (qCH23) was significant in the February replicate only (LOD=4.0; PVE=8.8%).

Two additional loci were detected in some of the replicates in the two-dimensional (2D) model-QTL model and the multiple-QTL model (MQM). A QTL on LG1 was significant in all replicates at 28.1-48.1 cM and a QTL on LG14 at 19.6 cM interacting with LG1 was significant in the February replicate. All loci from the single QTL model were significant except LG7 in the October replicate. MQM of the October replicate supported a single QTL model (LOD=21.5), however the December and February replicates supported a four-QTL (LOD=23.5) and five-QTL model with one interaction (LOD=27.2), respectively. Results of the MQM analysis across all three replicates in time indicate that the QTL on LG24 represents the major QTL for chilling response in this population.

Marker 938232 was estimated as the closest marker to the QTL peak on LG24 (qCH24). Analysis of the phenotype effects of the markers associated with this QTL indicated that F₂ individuals with two alleles from ‘CB15’ (AA) were the most chilling tolerant while those with one allele from each parent (AB) were the most sensitive as exemplified by the marker effect plots for marker 938232 (FIG. 12). The other markers linked to this QTL (qCH24) followed the same pattern, suggesting non-additive or dominant gene action for chilling sensitivity. Marker 930202 at 0.3 cM was estimated to be the QTL end-point in all three chilling assays. Marker 946237 at position 87.0 cM was estimated to be the nearest flanking marker of the other end-point in the October and February assay while marker 936090 at 65 cM was estimated to be the other end point in the December assay.

### Example 7

### QTL Identification for Aroma Compounds

Three significant QTL were detected for aroma compounds in the single-QTL model (FIGS. 13-14; Table 2). QTL were detected for estragole on LG1 (qEST1; LOD=19.2; PVE=35.9%) and LG3 (qEST3; LOD=12.5; PVE=25.2%) spanning from 13.3 to 18.7 cM and 65.2 to 70.3 cM, respectively, with the QTL on LG1 (qEST1) having the largest effect (FIG. 15). A QTL was detected on LG26 (LOD=8.0; PVE=16.9%) for eucalyptol spanning from 113.0 to 132.4 cM. No significant QTL was detected for linalool. The LOD thresholds (α=0.05) were determined to be 3.80, 3.66 and 3.78 for estragole, eucalyptol and linalool, respectively. No additional loci were detected as interacting with the significant loci in the 2D-QTL model or MQM. Furthermore, MQM refined that only the QTL on LG1 and not that on LG3 was significant for estragole.

Marker 765 was estimated as the closest marker to the QTL peak for estragole on LG1 (qEST1) (FIG. 15). Analysis of the phenotype effects of the markers associated with this QTL indicated that F₂ individuals with two alleles from ‘CB15’ (AA) had the highest levels of estragole while those with one allele from each parent (AB) had the lowest levels of estragole. The other markers linked to this QTL (qEST1) observe the same trend, suggesting non-additive gene action where the low estragole genotype (BB) is dominant. Marker 206 at 17.9 cM and marker 5502 at 58.6 cM were estimated to be the nearest flanking markers to the approximate end points of the QTL.

Marker 989212 was estimated as the closest physical marker to the QTL peak for eucalyptol on LG26 (qEUC26). The gene action is less clear for this QTL as the phenotypic effects were similar for the F₂ inheriting the AA genotype and the BB genotype. Marker 978874 at 113.0 cM and marker 989820 at 132.38 were estimated to be the nearest flanking markers to the approximate end points of the QTL.

### Example 8

### Gene Annotation

Several putative genes within the QTLs associated with cold response (qCH7, qCH13, qCH16, qCH23 and qCH24) were annotated with genes homologous to cold response and stress proteins in general (Table 3). Two annotations with functional responses to cold temperature were identified, including cold shock protein 1 on LG7 and putative transcriptional regulator UXT on LG23. Late embryogenesis abundant (LEA) proteins, 14-3-3 proteins, abscisic acid (ABA) related isoforms and binding factors, fatty acid desaturases (FADs) and proline related enzymes were annotated to the cold responsive QTLs and of particular interest for their relationship to cold stress [70-74]. Other annotations included 11 universal or general stress-response proteins on LG7, LG13, LG16, LG23 and LG24. Many putative genes on the QTLs associated with cold response were associated with regulator proteins including 512 annotations for kinases and 159 annotations for transcription factors. Many annotations were also associated with other related to cold response including 190 transport, 126 membrane and 33 heat shock proteins.

Putative genes that are homologous to terpene enzymatic proteins were identified on the QTLs associated with aroma compounds (qEST1 and qEUC26) as shown in Table 4. One gene annotated to LG1 as caffeic acid O-methyltransferase, which is a critical enzyme in the phenylpropanoid metabolic pathway [75]. Caffeoyl-CoA O-methyltransferase is also an important enzyme in phenylpropene synthesis for which 6 annotations were identified on LG1 and LG26 [76]. Annotations for 2 terpene synthases and 1 terpene cyclase/mutase family member were identified on LG26, which comprised a significant QTL for the monoterpene eucalyptol.

### Example 9

### New Chilling Tolerant Cultivars

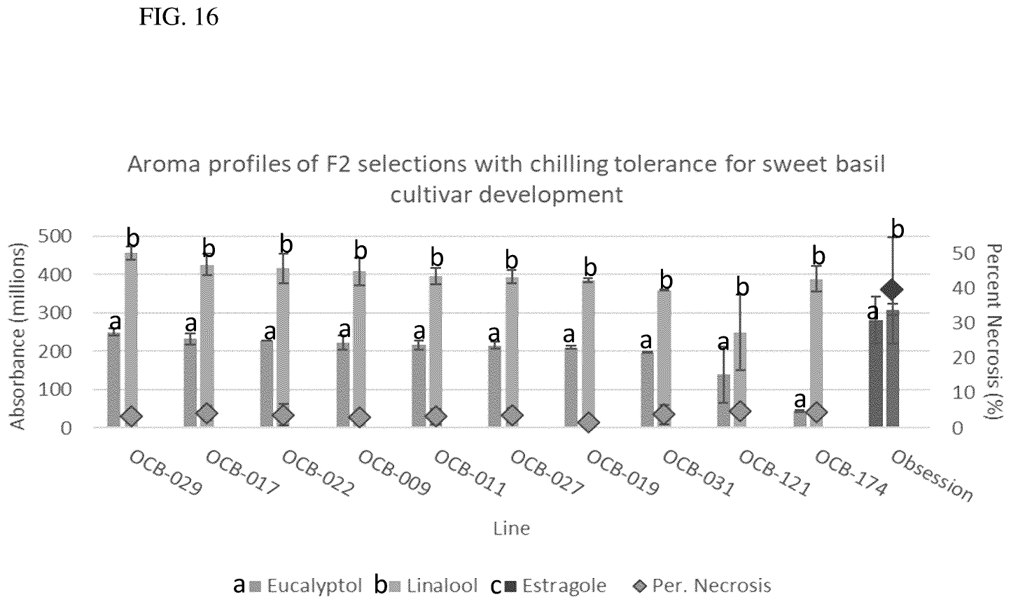

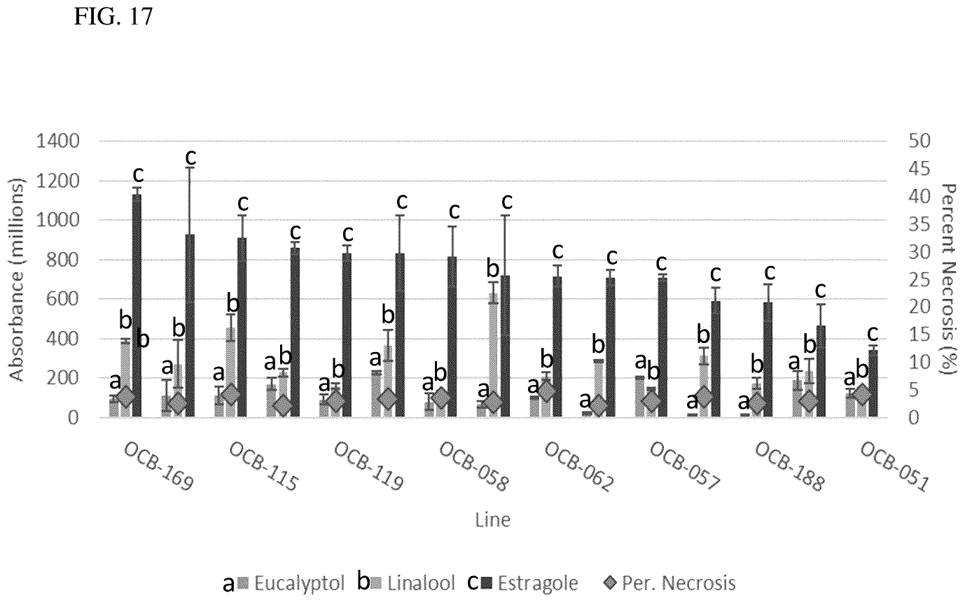

From the F2 population, 25 new sweet basil cultivars (‘OCB-029’, ‘OCB-017’, ‘OCB-022’, ‘OCB-009’, ‘OCB-011’, ‘OCB-027’, ‘OCB-019’, ‘OCB-031’, ‘OCB-121’, ‘OCB-174’, ‘OCB-169’, ‘OCB-153’, ‘OCB-115’, ‘OCB-120’, ‘OCB-119’, ‘OCB-067’, ‘OCB-058’, ‘OCB-086’, ‘OCB-062’, ‘OCB-117’, ‘OCB-057’, ‘OCB-126’, ‘OCB-188’, ‘OCB-129’, or ‘OCB-051’) were selected, based on their sweet basil (FIG. 16) or Thai aroma (FIG. 17) types and chilling tolerance. Sweet basil aroma types are characterized by high linalool content, a moderate eucalyptol content and low estragole 1 content while Thai basil aroma types are characterized by high estragole content and moderate to high linalool content. Chilling tolerance is broadly defined as the plants ability to survive low temperatures and is demonstrated by the individual plats demonstrating an average of less than 5% necrosis after refrigeration for 4-5 days at 3-5° C. S elections were also screened for their tolerance to basil downy mildew (BDM) in the greenhouse. Individuals with lower scores are tolerant and those with higher scores are susceptible to BDM.

As shown in Table 1, the new varieties had a mean necrosis value of <5% necrosis after refrigeration for 4-5 days at 3-5° C. (mean necrosis values of about 1.59%-4.83% were observed), indicating they all have chilling tolerance. In contrast, the parent ‘Rutgers Obsession DMR’ had a mean necrosis value of about 40%, indicating it is not chilling tolerant. Table 1 also provides the BDM score, an indicator of downy mildew tolerance. F2 basil lines were grown to maturity from cuttings in the greenhouse. Plants were inoculated with BDM, placed in the mist house overnight and screened for BDM severity using a visual scale (0-10). A score of 0 indicates resistance or no visible sporulation while a score of 10 indicates complete susceptibility or 100% sporulation. The BDM used for inoculum initially collected from summer field trials in NJ and maintained as live inoculum on plants in the greenhouse over the winter season. The aroma profile of each was characterized based on its mean and standard error (SE) estragole, eucalyptol and linalool content, and then assigned sweet basil or Thai basil aroma type (Table 1) (methods described in Example 1). No estragole or low levels of estragole were detected in a majority of F₂ individuals, including the 25 selected varieties (Table 1), suggesting that the gene action is likely non-additive or dominant for the low estragole phenotype. These results are supported by the marker effect plots (FIG. 15).

These 25 new varieties have not been observed under all possible environmental conditions to date. Accordingly, it is possible that the phenotype may vary somewhat with variations in the environment, such as temperature, light intensity, and day length, without, however, any variance in genotype. These new varieties provide chilling tolerant sweet basils. This combination of chilling tolerance and aroma characteristics do not yet exist in the market.

TABLE 1
Phenotype of 25 new basil varieties and parent ‘Rutgers Obsession DMR’

| Variety | Mean_Necrosis | BDM_Score | Mean_Estragole | Mean_Eucalyptol | Mean_Linalool |
|---|---|---|---|---|---|
| OCB-029 | 3.25 | 2.0 | NA | 249453046.3 | 456867563.3 |
| OCB-017 | 4.24 | 3.0 | NA | 232785355 | 426341066.3 |
| OCB-022 | 3.70 | 2.0 | NA | 227553871.3 | 416739724.3 |
| OCB-009 | 3.13 | 7.0 | NA | 222891712.3 | 408221086 |
| OCB-011 | 3.25 | 10.0 | NA | 215887043.7 | 395392196 |
| OCB-027 | 3.57 | 2.0 | NA | 215120005.7 | 393987382.7 |
| OCB-019 | 1.59 | 5.0 | NA | 210038258.7 | 384580297.7 |
| OCB-031 | 3.88 | 1.5 | NA | 196370814.3 | 359648667 |
| OCB-121 | 4.74 | 3.0 | NA | 138669473.7 | 248256606 |
| OCB-174 | 4.63 | NA | NA | 43562061.33 | 383967599.7 |
| Obsession | 39.44 | NA | NA | 281601576.7 | 308747005.3 |
| OCB-169 | 3.78 | NA | 1130052356 | 95665426.67 | 387397394.7 |
| OCB-153 | 2.49 | NA | 925837801.7 | 110719584 | 271261617 |
| OCB-115 | 4.09 | 6.0 | 909416099.7 | 110044386.3 | 456490541.3 |
| OCB-120 | 2.17 | 4.0 | 858214066.7 | 169246615 | 227433415.3 |
| OCB-119 | 2.97 | 1.0 | 832996916.7 | 90811792.67 | 157204730.7 |
| OCB-067 | 3.35 | 0.0 | 832766640.7 | 227350254.7 | 364590529 |
| OCB-058 | 3.59 | 8.0 | 815543298 | 79523504.33 | 116831582.7 |
| OCB-086 | 2.70 | 7.0 | 720020191.3 | 65379700 | 631377920.7 |
| OCB-062 | 4.83 | 5.0 | 714874201.3 | 101053934.3 | 209328274.3 |
| OCB-117 | 2.24 | 9.0 | 708686227.3 | 20745835.33 | 284922753.3 |
| OCB-057 | 2.90 | 9.0 | 706923531.7 | 200191119.7 | 146707722.7 |
| OCB-126 | 3.73 | 9.0 | 592451854 | 11554943.33 | 311923898.3 |
| OCB-188 | 2.49 | NA | 583012213.7 | 11615499.67 | 171968338.7 |
| OCB-129 | 2.88 | 2.0 | 464136466.7 | 189096485.3 | 232724806.3 |
| OCB-051 | 4.12 | 8.0 | 339677661 | 123436209 | 139063452.7 |

| Variety | SE_Necrosis | SE_Estragole | SE_Eucalyptol | SE_Linalool | Aroma_Type |
|---|---|---|---|---|---|
| OCB-029 | 1.68 | NA | 9413530.492 | 17240666.2 | Sweet |
| OCB-017 | 2.07 | NA | 14801153.83 | 27107975.6 | Sweet |
| OCB-022 | 3.02 | NA | 21270914.\grave{ }3842021 | 38957194.5 | Sweet |
| OCB-009 | 0.94 | NA | 19306464.42 | 35359348.9 | Sweet |
| OCB-011 | 2.15 | NA | 11926909.79 | 21843863.2 | Sweet |
| OCB-027 | 2.03 | NA | 9738534.047 | 17835902.8 | Sweet |
| OCB-019 | 0.80 | NA | 3336317.782 | 6110390.08 | Sweet |
| OCB-031 | 2.83 | NA | 1115901.73 | 2043748.67 | Sweet |
| OCB-121 | 1.81 | NA | 72798498.83 | 98214207.9 | Sweet |
| OCB-174 | 0.55 | NA | 3055948.304 | 33996000.5 | Sweet NI |
| Obsession | 15.13 | NA | 60957367.88 | 13942552.3 | |
| OCB-169 | 1.56 | 34267099.67 | 16284361.13 | 12015764.3 | Thai |
| OCB-153 | 0.66 | 339691874.3 | 78649679.48 | 119645287 | Thai |
| OCB-115 | 2.09 | 115314871.7 | 46186796.08 | 66939121.2 | Thai |
| OCB-120 | 1.46 | 29549842.49 | 31264131.63 | 20733492.6 | Thai |
| OCB-119 | 1.75 | 39874688.15 | 26862819.58 | 14329611.7 | Thai |
| OCB-067 | 1.68 | 190198367.3 | 7394896.481 | 79423529.8 | Thai |
| OCB-058 | 1.64 | 152767443.4 | 44525299.07 | 17898883.9 | Thai |
| OCB-086 | 1.24 | 305133258.7 | 17149399.03 | 54999406.8 | Thai |
| OCB-062 | 2.67 | 54526397.5 | 5588676.603 | 21402576.4 | Thai |
| OCB-117 | 1.54 | 38895889.38 | 3225614.055 | 6858424.14 | Thai |
| OCB-057 | 1.37 | 17024540.23 | 4828763.443 | 6076321.02 | Thai |
| OCB-126 | 1.39 | 64979027.72 | 2490933.115 | 42729882.8 | Thai |
| OCB-188 | 1.65 | 93256506.52 | 2705425.195 | 29066909 | Thai |
| OCB-129 | 1.34 | 109693486.4 | 47310362.18 | 61950029.4 | Thai |
| OCB-051 | 2.99 | 24178056.69 | 22431560.98 | 11824502.2 | Thai |

TABLE 2
Loci where LOD scores exceeded the LOD threshold (α = 0.05) in the single-QTL model.

| | | | | Peak position | | | | | | |
|---|---|---|---|---|---|---|---|---|---|---|
| | | | | | | PVE | Closest marker | | | Interval |
| Phenotype | Replicate | QTL | LG | cM | LOD | (%) | Marker | cM | LOD | (cM) |
| Chilling | October | qCH7 | 7 | 98.1 | 4.7 | 10.3 | 302350 | 98.1 | 4.7 | 93.0-106.0 |
| | December | | | 98.1 | 6.1 | 13.2 | 302350 | 98.1 | 6.1 | 93.0-102.5 |
| Chilling | October | qCH13 | 13 | 38.8 | 4.5 | 9.8 | 589396 | 38.8 | 4.5 | 10.9-41.1 |
| Chilling | February | qCH16 | 16 | 63.2 | 4.3 | 9.5 | 699095 | 63.2 | 4.3 | 51.7-74.0 |
| Chilling | February | qCH23 | 23 | 9.8 | 4.0 | 8.8 | 923381 | 9.8 | 4.0 | 0.2-15.3 |
| Chilling | October | qCH24 | 24 | 67.9 | 12.8 | 25.6 | 938232 | 67.9 | 12.8 | 0.3-78.3 |
| | December | | | 57.3 | 10.4 | 21.4 | 936090 | 64.8 | 10.0 | 0.3-64.8 |
| | February | | | 40.3 | 4.1 | 9.0 | 930202 | 0.3 | 3.6 | 0.3-65.3 |
| Estragole | NA | qEST1 | 1 | 42.7 | 19.2 | 35.9 | 765 | 42.7 | 19.2 | 39.1-44.1 |
| Estragole | NA | qEST3 | 3 | 68.7 | 12.5 | 25.2 | 120144 | 68.7 | 12.5 | 65.3-70.3 |
| Eucalyptol | NA | qEUC26 | 26 | 130.0 | 8.0 | 16.9 | 989820 | 132.4 | 7.6 | 113.0-132.4 |

Peak position was determined from a genome scan with a single-QTL model using the Haley-Knott regression mapping method and 1,000 permutations. Closest markers and confidence intervals were determined with 95% bayes credible intervals. Peak position (cM) and markers represent those with the highest LOD). The LOD thresholds (α = 0.05) were determined to be 3.70, 3.96 and 3.81 for the chilling assay replicates in October, December and February, respectively, 3.80 for estragole and 3.66 for eucalyptol.

TABLE 3
Genome annotations of interest within QTLs associated with cold response.

| LG | Homologous gene | Function | Species | AED | Uniprot Accession |
|---|---|---|---|---|---|
| 7 | Cold shock protein 1 | NA | Striga asiatica | 0.07 | A0A5A7RB26 |
| 7 | Universal stress protein 27 | NA | Salvia miltiorrhiza | 0.04 | A0A290YXU3 |
| 7 | Stress-response A/B barrel | NA | Handroanthus | 0.39 | A0A2G9I0I6 |
| | domain-containing protein | | impetiginosus | | |
| 7 | Stress response protein | NA | Sesamum indicum | 0.27 | A0A6I9UJN7 |
| | nst1 | | | | |
| 7 | LEA_2 domain-containing | NA | Salvia splendens | 0.26 | A0A4D8ZMQ8 |
| | protein | | | | |
| 7 | 14-3-3-like protein 16R | NA | Sesamum indicum | 0.04 | A0A6I9U980 |
| 7 | Protein ABA DEFICIENT | NA | Sesamum indicum | 0.02 | A0A6I9U8G4 |
| | 4 chloroplastic-like | | | | |
| | isoform | | | | |
| 7 | ABA responsive element | NA | Salvia splendens | 0.35 | A0A4D9BA86 |
| | binding factor | | | | |
| 7 | Proline dehydrogenase | NA | Salvia splendens | 0.07 | A0A4D8ZXX4 |
| 7 | Omega-3 fatty acid | NA | Perilla frutescens | 0.00 | O04807 |
| | desaturase | | | | |
| 7 | FAD-binding FR-type | NA | Vitis vinifera | 0.29 | E0CUW8 |
| | domain-containing protein | | | | |
| 7 | FAD-binding FR-type | NA | Salvia splendens | 0.06 | A0A4D9AH61 |
| | domain-containing protein | | | | |
| 7 | FAD-binding FR-type | NA | Salvia splendens | 0.38 | A0A4D9AP55 |
| | domain-containing | | | | |
| 13 | Universal stress protein 23 | NA | NA | 0.25 | A0A290YXT7 |
| 13 | Proline iminopeptidase | NA | Handroanthus | 0.19 | A0A2G9GH38 |
| | | | impetiginosus | | |
| 13 | Omega-3 fatty acid | NA | Perilla frutescens | 0.25 | A0A2H4MCG8 |
| | desaturase | | | | |
| 16 | Universal stress protein 7 | protein kinase | NA | 0.03 | A0A290YXQ1 |
| | | activity; ATP | | | |
| | | binding; protein | | | |
| | | phosphorylation | | | |
| 16 | Universal stress protein 22 | NA | Salvia miltiorrhiza | 0.02 | A0A290YXT6 |
| 16 | Universal stress protein 19 | NA | Salvia miltiorrhiza | 0.02 | A0A290YXS5 |
| 16 | Universal stress protein 21 | NA | Salvia miltiorrhiza | 0.01 | A0A290YXU1 |
| 16 | Universal stress protein 8 | NA | Salvia miltiorrhiza | 0.14 | A0A290YXN9 |
| 16 | LEA_2 domain-containing | NA | Erythranthe guttata | 0.17 | A0A022PQW9 |
| | protein | | | | |
| 16 | LEA_2 domain-containing | NA | Salvia splendens | 0.02 | A0A4D8YFU8 |
| | protein | | | | |
| 16 | ABA-responsive element | NA | Salvia miltiorrhiza | 0.40 | A0A2U9Q8N8 |
| | ABRE-binding | | | | |
| | transcription factor1 | | | | |
| 16 | FAD synthase isoform X1 | NA | Perilla frutescens | 0.11 | A0A6I9T1J8 |
| 23 | Putative transcriptional | protein folding; | NA | 0.16 | A0A2G9GFB7 |
| | regulator UXT | response to cold | | | |
| 23 | Universal stress protein 13 | NA | Salvia miltiorrhiza | 0.12 | A0A290YXS7 |
| 23 | 14-3-3 protein 9 | NA | Sesamum indicum | 0.38 | A0A6I9UP33 |
| 23 | ABA responsive element | NA | Salvia splendens | 0.18 | A0A4D9BM39 |
| | binding factor | | | | |
| 24 | Universal stress protein 13 | NA | Salvia miltiorrhiza | 0.08 | A0A290YXS7 |
| 24 | 14-3-3 protein 9 | NA | Sesamum indicum | 0.38 | A0A6I9UP33 |
| 24 | ABA responsive element | NA | Salvia splendens | 0.17 | A0A4D9BM39 |
| | binding factor | | | | |
| 24 | Omega-3 fatty acid | NA | Perilla frutescens | 0.11 | A0A2H4MEV2 |
| | desaturase | | | | |

Putative genes of interest within significant QTL were those homologous to genes associated with cold or stress. NA indicates information not available. LG = linkage group.

TABLE 4
Genome annotations of interest within QTLs associated with aroma.

| | | QTL | | | Uniprot |
|---|---|---|---|---|---|
| LG | Homologous gene | Association | Species | AED | Accession |
| 1 | Caffeic acid O- | Estragole | Catalpa | 0.05 | A0A411ASI0 |
| | methyltransferase | | bungei | | |
| 1 | Caffeoyl-CoA O- | Estragole | Salvia | 0.17 | A0A4D9BQK0 |
| | methyltransferase | | splendens | | |
| 1 | Caffeoyl-CoA O- | Estragole | Salvia | 0.45 | A0A6G5RUA9 |
| | methyltransferase | | dorisiana | | |
| 26 | Terpene synthase | Eucalyptol | Prunella | 0.08 | A0A6B7L8X6 |
| | | | vulgaris | | |
| 26 | Terpene synthase | Eucalyptol | Scutellaria | 0.25 | A0A6B7LU35 |
| | | | barbata | | |
| 26 | Terpene cyclase/ | Eucalyptol | Ocimum | 0.03 | A0A0D3L308 |
| | mutase family | | basilicum | | |
| | member | | | | |
| 26 | Caffeoyl-CoA O- | Eucalyptol | Erythranthe | 0.23 | A0A022QTM1 |
| | methyltransferase | | guttata | | |
| 26 | Caffeoyl-CoA O- | Eucalyptol | Salvia | 0.46 | A0A4D9ASU9 |
| | methyltransferase | | splendens | | |
| 26 | Caffeoyl-CoA O- | Eucalyptol | Erythranthe | 0.01 | A0A022QTM1 |
| | methyltransferase | | guttata | | |
| 26 | Caffeoyl-CoA O- | Eucalyptol | Daucus | 0.20 | A0A166C3S9 |
| | methyltransferase | | carota | | |
| | | | subsp. | | |
| | | | Sativus | | |

Putative genes of interest within significant QTL were those homologous to genes associated with terpene biochemical pathways. NA indicates information not available. LG = linkage group.

### REFERENCES

1. Simon et al., Basil: A source of aroma compounds and a popular culinary and ornamental herb. In: Janick J, editor. Perspectives on new crops and new uses. Alexandria, VA: ASHS Press; 1990. p. 499-505.
2. More A. Basil Leaves Market 2022 Is Expected Growth of USD 63 mn by 2027|Industry Size, Share, Revenue, Top Players, Region|Research Report by Absolute Reports: Absolute Reports Pvt Ltd; 2022. Available from: https://www.globenewswire.com/en/news-release/2022/01/12/2365772/0/en/Basil-Leaves-Market-2022-Is-Expected-Growth-of-USD-63mn-by-2027-Industry-Size-Share-Revenue-Top-Players-Region-Research-Report-by-Absolute-Reports.html.
3. Edet O U, Aikpokpodion P O. Karyotype analysis of Ocimum basilicum in south-eastern Nigeria. American Journal of Plant Sciences. 2014; 5(1):126-31. doi: 10.4236/ajps.2014.51016.
4. Kohsla M K. Chromosome meiotic behaviour and ploidy nature in genus Ocimum (Lamiaceae), “Sanctum Group”. Cytologia. 1989; 54:223-9.
5. Singh T P. Karyomorphological studies in Ocimum basilicum L. utilized in an effort to solve the taxonomic status of O. carnosum. The Journal of Indian Botanical Society. 1986; 66:402-7.
6. Truta E, Zamfirache M M. Preliminary cytogenetical investigation in two common basil varieties. Analele Stiintifice ale Universitatii “Al I Cuza” Din Iasi (Serie Noua) Sectiunea 2a Genetica si Biologie Moleculara. 2013; 14(2):29-34.
7. Pushpangadan P, Sobti S N. Cytogenetical studies in the genus Ocimum I. Origin of O. americanum, cytotaxonomical and experimental proof. Cytologia. 1982; 47:575-83.
8. Biswas T, Mukhopadhyay S. Comparison of different cytological parameters of three species of Ocimum. Bangladesh Journal of Botany Short Communication. 2020; 49(4):1163-7.
9. Carović-Stanko et al., Genetic relations among basil taxa (Ocimum L.) based on molecular markers, nuclear DNA content, and chromosome number. Plant Systematics and Evolution. 2010; 285(1-2):13-22. doi: 10.1007/s00606-009-0251-z.
10. Gonda et al., The genome sequence of tetraploid sweet basil, Ocimum basilicum L., provides tools for advanced genome editing and molecular breeding. DNA Research. 2020; 27(5):1-10. doi: 10.1093/dnares/dsaa027. PubMed PMID: 33340318; PubMed Central PMCID: PMCPMC7758295.
11. Paton A, Putievsky E. Taxonomic problems and cytotaxonomic relationships between and within varieties of Ocimum basilicum and related species (Labiatae). Kew Bulletin. 1996; 51(3):509-24.
12. Mukherjee M, Datta A K. Secondary chromosome associations in Ocimum basilicum L. and Ocimum tenuiflorum L. Cytologia. 2005; 70(2).
13. Dash et al., Conventional and fluorescent karyotype analysis of Ocimum spp. Cytologia. 2017; 82(4):429-34. doi: 10.1508/cytologia.82.429.
14. Rewers M, Jedrzejczyk I. Genetic characterization of Ocimum genus using flow cytometry and inter-simple sequence repeat markers. Industrial Crops and Products. 2016; 91:142-51. doi: 10.1016/j.indcrop.2016.07.006.
15. Koroch et al., Estimation of nuclear DNA content of cultivated Ocimum species by using flow cytometry. Israel Journal of Plant Sciences. 2010; 58(3):183-9. doi: 10.1560/ijps.59.3-4.183.
16. Pyne et al., A first linkage map and downy mildew resistance QTL discovery for sweet basil (Ocimum basilicum) facilitated by double digestion restriction site associated DNA sequencing (ddRADseq). PLoS One. 2017; 12(9):e0184319. Epub 2017/09/19. doi: 10.1371/journal.pone.0184319. PubMed PMID: 28922359; PubMed Central PMCID: PMCPMC5603166.
17. Zhan et al., De novo assembly and analysis of the transcriptome of Ocimum americanum var. pilosum under cold stress. BMC Genomics. 2016; 17(209):1-12. Epub 20160309. doi: 10.1186/s12864-016-2507-7. PubMed PMID: 26955811; PubMed Central PMCID: PMCPMC4784345.
18. Begum F U. The improvement of chilling tolerance in chilling sensitive basil (Ocimum basilicum var Marian) [Doctoral thesis]. London: oyal Holloway University of London; 2017.
19. Gonda et al., Genome-based high-resolution mapping of fusarium wilt resistance in sweet basil. Plant Science. 2022; 321:1-10. Epub 20220511. doi: 10.1016/j.plantsci.2022.111316. PubMed PMID: 35696916.
20. Mattera et al., Ocimum basilicum ‘RUSB22’ reference genome. US Basil Consortium2023.
21. Patel et al., Tepper B J. Descriptive aroma profiles of fresh sweet basil cultivars (Ocimum spp.): Relationship to volatile chemical composition. Journal of Food Science. 2021; 86(7):3228-39. Epub 20210623. doi: 10.1111/1750-3841.15797. PubMed PMID: 34160060.
22. Bernhardt et al., Comparison of different Ocimum basilicum L. gene bank accessions analyzed by GC-MS and sensory profile. Industrial Crops and Products. 2015; 67:498-508. doi: 10.1016/j.indcrop.2015.01.013.
23. Vieira R F, Simon J E. Chemical characterization of basil (Ocimum spp.) based on volatile oils. Flavour and Fragrance Journal. 2006; 21(2):214-21. doi: 10.1002/ffj.1513.
24. Hammock et al., Narrowband blue and red LED supplements impact key flavor volatiles in hydroponically grown basil across growing seasons. Frontiers in Plant Science. 2021; 12:1-15. Epub 20210226. doi: 10.3389/fpls.2021.623314. PubMed PMID: 33719295; PubMed Central PMCID: PMCPMC7952523.
25. Charles D J, Simon J E. Comparison of extraction methods for the rapid determination of essential oil content and composition of basil. Journal of the American Society for Horticultural Science. 1990; 115(3):458-62.
26. Drew et al., A comparison of headspace solid-phase microextraction and classic hydrodistillation for the identification of volatile constituents from Thapsia spp. provides insights into guaianolide biosynthesis in Apiaceae. Phytochemical Analysis. 2012; 23(1):44-51. Epub 20110525. doi: 10.1002/pca.1323. PubMed PMID: 21618308.
27. Cavalli et al., Comparison of static headspace, headspace solid phase microextraction, headspace sorptive extraction, and direct thermal desorption techniques on chemical composition of French olive oils. Journal of Agricultural and Food Chemistry. 2003; 51:7709-16.
28. Miller M E, Stuart J D. Comparison of gas-sampled and SPME-sampled static deadspace for the determination of volatile flavor components. Analytical Chemistry. 1999; 71:23-7.
29. Bartelt R J. Calibration of a commercial solid-phase microextraction device for measuring headspace concentrations of organic volatiles. Analytical Chemistry. 1997; 69:364-72.
30. Kliminkovi et al., Aroma profiles of five basil (Ocimum basilicum L.) cultivars grown under conventional and organic conditions. Food Chemistry. 2008; 107(1):464-72. doi: 10.1016/j.foodchem.2007.07.062.
31. Cozzolino R, Pace B, Cefola M, Martignetti A, Stocchero M, Fratianni F, et al. Assessment of volatile profile as potential marker of chilling injury of basil leaves during postharvest storage. Food Chem. 2016; 213:361-8. Epub 20160630. doi: 10.1016/j.foodchem.2016.06.109. PubMed PMID: 27451192.
32. Hammock H A. The impact of blue and red LED lighting on biomass accumulation, flavor volatile production, and nutrient uptake in hydroponically grown Genovese basil [Masters thesis]. Knoxville: University of Tennessee; 2018.
33. Woronuk et al., Biosynthesis and therapeutic properties of Lavandula essential oil constituents. Planta Medica. 2011; 77(1):7-15. Epub 20100721. doi: 10.1055/s-0030-1250136. PubMed PMID: 20665367.
34. Eisenbrand et al., FEMA GRAS assessment of natural flavor complexes: Eucalyptus oil and other cyclic ether-containing flavoring ingredients. Food and Chemical Toxicology. 2021; 155:1-22. Epub 20210701. doi: 10.1016/j.fct.2021.112357. PubMed PMID: 34217737.
35. Gang et al., An investigation of the storage and biosynthesis of phenylpropenes in sweet basil. Plant Physiology. 2001; 125:539-55.
36. Paton et al., Ocimum: An overview of classification and relationships. In: Hiltunen R, Holm Y, editors. Basil. London: CRC Press; 1999. p. 36.
37. Harley et al., Labiatae. In: Kadereit J W, editor. Flowering Plants—Dicotyledons. VII. Berlin, Heidelberg: Springer; 2004. p. 167-275.
38. Cantwell M I, Reid M S. Postharvest Physiology and Handling of Fresh Culinary Herbs. Journal of Herbs, Spices & Medicinal Plants. 1993; 1(3):93-127. doi: 10.1300/J044v01n03_09.
39. Lange D L, Cameron A C. Pre- and postharvest temperature conditioning of greenhouse-grown sweet basil. HortScience. 1997; 32(1):114-6. doi: 10.21273/hortsci.32.1.114.
40. Lange D D, Cameron A C. Postharvest Shelf Life of Sweet Basil (Ocimum basilicum). HortScience. 1994; 29(2):102-3. doi: 10.21273/hortsci.29.2.102.
41. Aharoni et al., Reducing chilling injury and decay in stored sweet basil. Israel Journal of Plant Sciences. 2010; 58(3):167-81. doi: 10.1560/ijps.58.3-4.167.
42. Ribeiro P, Simon J E. Breeding sweet basil for chilling tolerance. Alexandria, VA: ASHS Press; 2007.
43. Brindisi et al., Rapid screening methods to identify chilling tolerance in sweet basil (Ocimum basilicum L.). Journal of Medicinally Active Plants. 2021; 10(1):1-10. doi: 10.7275/2g05-ea63.
44. Brindisi L J, Simon J E. Preharvest and postharvest techniques that optimize the shelf life of fresh basil (Ocimum basilicum L.): a review. Frontiers in Plant Science. 2023; 14:1-15. https://doi.org/10.3389/fpls.2023.1237577 PMID: 37745993
45. Jensen et al., Spectral quality of supplemental LED grow light permanently alters stomatal functioning and chilling tolerance in basil (Ocimum basilicum L.). Scientia Horticulturae. 2018; 227:38-47. doi: 10.1016/j.scienta.2017.09.011.
46. Larsen et al., Response of basil growth and morphology to light intensity and spectrum in a vertical farm. Frontiers in Plant Science. 2020; 11:1-16. Epub 20201204. doi: 10.3389/fpls.2020.597906. PubMed PMID: 33424894; PubMed Central PMCID: PMCPMC7793858.
47. Eskin N A M, Hoehn E. Fruits and Vegetables. In: Michael Eskin N A, Shahidi F, editors. Biochemistry of Foods. 3 ed: Academic Press; 2013. p. 49-126.
48. Obořil M. Quantification of Leaf Necrosis by Biologically Inspired Algorithms. 2017.
49. Lobet G, Draye X, Pdrilleux C. An online database for plant image analysis software tools. Plant Methods. 2013; 9(38):1-7.
50. Adams R P. Identification of essential oil components by gas chromatography/mass spectrometry 4.1 ed. Carol Stream, Illinois: Allured publishing; 2017.
51. Team R C. R: A language and environment for statistical computing Vienna, Austria: R Foundation for Statistical Computing; 2020. Available from: https://www.R-project.org/.
52. Mangiafico S S. Summary and Analysis of Extension Program Evaluation in R 2016. Available from: rcompanion.org/handbook.
53. Poland et al., Development of high-density genetic maps for barley and wheat using a novel two-enzyme genotyping-by-sequencing approach. PLoS One. 2012; 7(2):e32253. Epub 2012/03/06. doi: 10.1371/journal.pone.0032253. PubMed PMID: 22389690; PubMed Central PMCID: PMCPMC3289635.
54. Catchen et al., Stacks: an analysis tool set for population genomics. Mol Ecol. 2013; 22(11):3124-40. Epub 2013/05/25. doi: 10.1111/mec.12354. PubMed PMID: 23701397; PubMed Central PMCID: PMCPMC3936987.
55. Li H. Aligning sequence reads, clone sequences and assembly contigs with BWA-MEM. In: MIT BIoHa, editor. Cambridge, MA: arXiv:1303.3997v1 [q-bio.GN]; 2013. p. 1-3.
56. Danecek P, Bonfield J K, Liddle J, Marshall J, Ohan V, Pollard M O, et al. Twelve years of SAMtools and BCFtools. Gigascience. 2021; 10(2):1-4. Epub 2021/02/17. doi: 10.1093/gigascience/giab008. PubMed PMID: 33590861; PubMed Central PMCID: PMCPMC7931819.
57. Broman K W, Wu H, Sen S, Churchill G A. R/qtl: QTL mapping in experimental crosses. Bioinformatics. 2003; 19(7):889-90. Epub 2003/05/02. doi: 10.1093/bioinformatics/btg112. PubMed PMID: 12724300.
58. Team R. RStudio: Integrated Development for R Boston, MA: RStudio, Inc.; 2019. Available from: http://www.rstudio.com/.
59. Cantarel B L, Korf I, Robb S M, Parra G, Ross E, Moore B, et al. MAKER: an easy-to-use annotation pipeline designed for emerging model organism genomes. Genome Research. 2008; 18(1):188-96. Epub 20071119. doi: 10.1101/gr.6743907. PubMed PMID: 18025269; PubMed Central PMCID: PMCPMC2134774.
60. Campbell M S, Holt C, Moore B, Yandell M. Genome Annotation and Curation Using MAKER and MAKER-P. Current Protocols in Bioinformatics. 2014; 48:1-39. Epub 20141212. doi: 10.1002/0471250953.bi0411s48. PubMed PMID: 25501943; PubMed Central PMCID: PMCPMC4286374.
61. Towns J, Cockerill T, Dahan M, Foster I, Gaither K, Grimshaw A, et al. XSeDe: Accelerating scientific discovery. Computing in Science & Engineering. 162014. p. 62-74.
62. Smit A, Hubley R, Green P. RepeatMasker: Institute for Systems Biology; 2015. Available from: www.repeatmasker.org.
63. Han Y, Wessler S R. MITE-Hunter: a program for discovering miniature inverted-repeat transposable elements from genomic sequences. Nucleic Acids Research. 2010; 38(22):1-8. Epub 20100929. doi: 10.1093/nar/gkg862. PubMed PMID: 20880995; PubMed Central PMCID: PMCPMC3001096.
64. Allen K S, DeIulio G A, Pyne R, Maman J, Guo L, Wick R L, et al. Identification of novel basil downy mildew resistance genes using de novo comparative transcriptomics. 2022.
65. UniProt C. UniProt: the Universal Protein Knowledgebase in 2023. Nucleic Acids Research. 2023; 51(D1):D523-D31. doi: 10.1093/nar/gkac1052. PubMed PMID: 36408920; PubMed Central PMCID: PMCPMC9825514.
66. Korf I. Gene finding in novel genomes. BMC Bioinformatics. 2004; 5:1-9.
67. Araujo F A, Barh D, Silva A, Guimaraes L, Ramos R T J. GO FEAT: a rapid web-based functional annotation tool for genomic and transcriptomic data. Scientific Reports. 2018; 8(1):1794. Epub 20180129. doi: 10.1038/s41598-018-20211-9. PubMed PMID: 29379090; PubMed Central PMCID: PMCPMC5789007.
68. Toronen et al., PANNZER2: a rapid functional annotation web server. Nucleic Acids Research. 2018; 46(W1):W84-W8. doi: 10.1093/nar/gky350. PubMed PMID: 29741643; PubMed Central PMCID: PMCPMC6031051.
69. Li H. Exploring single-sample SNP and INDEL calling with whole-genome de novo assembly. Bioinformatics. 2012; 28(14):1838-44. Epub 20120507. doi: 10.1093/bioinformatics/bts280. PubMed PMID: 22569178; PubMed Central PMCID: PMCPMC3389770.
70. Huang et al., ABA Is involved in regulation of cold stress response in bermudagrass. Frontiers in Plant Science. 2017; 8:1613. Epub 20171013. https://doi.org/10.3389/fpls.2017.01613 PMID: 29081782; PubMed Central PMCID: PMC5645512.
71. Yu et al., ABA enhanced cold tolerance of wheat ‘dn1’ via increasing ROS scavenging system. Plant Signaling & Behavior. 2020; 15(8):1780403. Epub 20200703. https://doi.org/10.1080/15592324.2020.1780403 PMID: 32619128; PubMed Central PMCID: PMC8570709.
72. Kosova et al., COR/LEA proteins as indicators of frost tolerance in Triticeae: A comparison of controlled versus field conditions. Plants. 2021; 10(4). Epub 20210416. https://doi.org/10.3390/plants10040789 PMID: 33923804; PubMed Central PMCID: PMC8073581.
73. Liu et al., Plasma membrane CRPK1-mediated phosphorylation of 14-3-3 proteins induces their nuclear import to fine-tune CBF signaling during cold response. Molecular Cell. 2017; 66(1):117-28 e5. Epub 20170323. https://doi.org/10.1016/j.molcel.2017.02.016 PMID:28344081.
74. Visconti et al., Overexpression of 14-3-3 proteins enhances cold tolerance and increases levels of stress-responsive proteins of Arabidopsis plants. Plant Science. 2019; 289:110215. Epub 20190928. https://doi.org/10.1016/j.plantsci.2019.110215 PMID:31623776.
75. Liang et al., Identification and functional analysis of the caffeic acid o-methyltransferase (COMT) gene family in rice (Oryza sativa L.). International Journal of Molecular Sciences. 2022; 23(15). Epub 20220731. https://doi.org/10.3390/ijms23158491 PMID: 35955626; PubMed Central PMCID: PMC9369235.
76. Ji et al., Caffeoyl-coenzyme A O-methyltransferase mediates regulation of carbon flux fluctuations during phenylpropenes and lignin biosynthesis in the vegetative organ roots of Asarum sieboldii Miq. Plant Physiology and Biochemistry. 2023; 201:107855. Epub 20230705. https://doi.org/10.1016/j.plaphy.2023.107855 PMID: 37433236.
77. Anderson S L 2nd, Mahan A L, Murray S C, Klein P E. Four parent maize (FPM) population: Effects of mating designs on linkage disequilibrium and mapping quantitative traits. Plant Genome. 2018; 11(2):1-17. https://doi.org/10.3835/plantgenome2017.11.0102 PMID: 30025026.
78. Whibley A, Kelley J L, Narum S R. The changing face of genome assemblies: Guidance on achieving high-quality reference genomes. Molecular Ecology Resources. 2021; 21(3):641-52. Epub 20210120. https://doi.org/10.1111/1755-0998.13312 PMID: 33326691.
79. Bourke et al., Partial preferential chromosome pairing is genotype dependent in tetraploid rose. the Plant Journal. 2017; 90(2):330-43. Epub 20170320. doi: 10.1111/tpj.13496. PubMed PMID: 28142191.
80. Zeng et al., Understanding the biosyntheses and stress response mechanisms of aroma compounds in tea (Camellia sinensis) to safely and effectively improve tea aroma. Critical Reviews in Food Science and Nutrition. 2019; 59(14):2321-34. Epub 20181002. doi: 10.1080/10408398.2018.1506907. PubMed PMID: 30277806.
81. Xie et al., A systems biology investigation of the MEP/terpenoid and shikimate/phenylpropanoid pathways points to multiple levels of metabolic control in sweet basil glandular trichomes. the Plant Journal. 2008; 54(3):349-61. Epub 20080131. doi: 10.1111/j.1365-313X.2008.03429.x. PubMed PMID: 18248593.
82. Dar A et al., The FAD2 gene in plants: Occurrence, regulation, and role. Frontiers in Plant Science. 2017; 8:1789. Epub 20171018. https://doi.org/10.3389/fpls. 2017.01789 PMID: 29093726; PubMed Central PMCID: PMC5651529.
83. Wang et al., GmFAD3A, A omega-3 fatty acid desaturase gene, enhances cold tolerance and seed germination rate under low temperature in rice. Intermolecular Journal of Molecular Sciences. 2019; 20(15). Epub 20190803. https://doi.org/10.3390/ijms20153796 PMID: 31382584; PubMed Central PMCID: PMC6696117.
84. Yan et al., Parallels between natural selection in the cold-adapted crop-wild relative Tripsacum dactyloides and artificial selection in temperate adapted maize. the Plant Journal. 2019; 99(5):965-77. Epub 20190626. https://doi.org/10.1111/tpj.14376 PMID: 31069858.
85. Choi et al., ABFs, a family of ABA-responsive element binding factors. J Biol Chem. 2000; 275(3):1723-30. https://doi.org/10.1074/jbc.275.3.1723 PMID: 10636868.
86. Raza A, Charagh S, Abbas S, Hassan M U, Saeed F, Haider S, et al. Assessment of proline function in higher plants under extreme temperatures. Plant Biol (Stuttg). 2023; 25(3):379-95. Epub 20230227. https://doi.org/10.1111/plb.13510 PMID: 36748909.
87. Praat et al., Protein kinase and phosphatase control of plant temperature responses. Journal of Experimental Botany. 2021; 72(21):7459-73. Epub 20210720. https://doi.org/10.1093/jxb/erab345 PMID: 34283227.
88. Dixit et al., Action of multiple intra-QTL genes concerted around a co-localized transcription factor underpins a large effect QTL. Scientific Reports. 2015; 5:1-16. Epub 20151028. doi: 10.1038/srep15183. PubMed PMID: 26507552; PubMed Central PMCID: PMCPMC4623671.
89. Hannah et al., A global survey of gene regulation during cold acclimation in Arabidopsis thaliana. PLoS Genetics. 2005; 1(2):0179-96. Epub 20050819. https://doi.org/10.1371/journal.pgen.0010026 PMID: 16121258; PubMed Central PMCID: PMC1189076.
90. Takahashi et al., Plant plasma membrane proteomics for improving cold tolerance. Frontiers in Plant Science. 2013; 4(90):1-5. Epub 20130417. https://doi.org/10.3389/fpls.2013.00090 PMID: 23616787; PubMed Central PMCID: PMC3628364.
91. Ul et al., Heat shock proteins: Dynamic biomolecules to counter plant biotic and abiotic stresses. International Journal of Molecular Sciences. 2019; 20(21):1-31. Epub 20191025. https://doi.org/10.3390/ijms20215321 PMID: 31731530; PubMed Central PMCID: PMC6862505.
92. Huang et al., Effect of eugenol fumigation treatment on chilling injury and CBF gene expression in eggplant fruit during cold storage. Food Chemistry. 2019; 292:143-50. Epub 20190417. doi: 10.1016/j.foodchem.2019.04.048. PubMed PMID: 31054659.
93. Zhao et al., Eugenol functions as a signal mediating cold and drought tolerance via UGT71A59-mediated glucosylation in tea plants. The Plant Journal. 2022; 109(6):1489-506. Epub 2021/12/22. doi: 10.1111/tpj.15647. PubMed PMID: 34931743.
94. Dhawan et al., A cold-tolerant evergreen interspecific hybrid of Ocimum kilimandscharicum and Ocimum basilicum: analyzing trichomes and molecular variations. Protoplasma. 2016; 253(3):845-55. Epub 20150709. https://doi.org/10.1007/s00709-015-0847-9 PMID: 26156173.95. Extension NCS. Ocimum kilimandscharicum x basilicum: N.C. State Cooperative Extension; [cited 2022 Oct. 10, 2022]. Available from: https://plants.ces.ncsu.edu/plants/ocimum-kilimandscharicum-x-basilicum/.

In view of the many possible embodiments to which the principles of the disclosure may be applied, it should be recognized that the illustrated embodiments are only examples of the disclosure and should not be taken as limiting the scope of the invention. Rather, the scope of the invention is defined by the following claims. We therefore claim as our invention all that comes within the scope and spirit of these claims.

We claim:

1. A plant of basil variety ‘OCB-029’, ‘OCB-017’, ‘OCB-022’, ‘OCB-009’, ‘OCB-011’, ‘OCB-027’, ‘OCB-019’, ‘OCB-031’, ‘OCB-121’, ‘OCB-174’, ‘OCB-169’, ‘OCB-153’, ‘OCB-115’, ‘OCB-120’, ‘OCB-119’, ‘OCB-067’, ‘OCB-058’, ‘OCB-086’, ‘OCB-062’, ‘OCB-117’, ‘OCB-057’, ‘OCB-126’, ‘OCB-188’, ‘OCB-129’, ‘OCB-051’, or ‘CB15’, wherein representative seed of said variety have been deposited under American Type Culture Collection (ATCC) Accession No. ______, respectively.

2. A plant part of the plant of claim 1, wherein the plant part comprises
an embryo of said plant, or
at least one cell of said plant.

3. The plant part of claim 2, wherein the plant part is pollen, a meristem, a cell, an ovule, a leaf, a root, a root tip, a pistil, an anther, a protoplast, or a cotyledon.

4. A tissue culture produced from the protoplast or the cell of claim 3.

5. The tissue culture of claim 4, wherein the cell or protoplast is produced from a leaf, stem, protoplast, pollen, ovule, cotyledon, hypocotyl, meristematic cell, root, root tip, pistil, anther, flower, seed, shoot, stein, pod or petiole.

6. A basil plant regenerated from the tissue culture of claim 4, wherein the regenerated sweet basil plant comprises all of the physiological and morphological characteristics of the basil variety ‘OCB-029’, ‘OCB-017’, ‘OCB-022’, ‘OCB-009’, ‘OCB-011’, ‘OCB-027’, ‘OCB-019’, ‘OCB-031’, ‘OCB-121’, ‘OCB-174’, ‘OCB-169’, ‘OCB-153’, ‘OCB-115’, ‘OCB-120’, ‘OCB-119’, ‘OCB-067’, ‘OCB-058’, ‘OCB-086’, ‘OCB-062’, ‘OCB-117’, ‘OCB-057’, ‘OCB-126’, ‘OCB-188’, ‘OCB-129’, ‘OCB-051’, or ‘CB15’.

7. A seed of basil variety ‘OCB-029’, ‘OCB-017’, ‘OCB-022’, ‘OCB-009’, ‘OCB-011’, ‘OCB-027’, ‘OCB-019’, ‘OCB-031’, ‘OCB-121’, ‘OCB-174’, ‘OCB-169’, ‘OCB-153’, ‘OCB-115’, ‘OCB-120’, ‘OCB-119’, ‘OCB-067’, ‘OCB-058’, ‘OCB-086’, ‘OCB-062’, ‘OCB-117’, ‘OCB-057’, ‘OCB-126’, ‘OCB-188’, ‘OCB-129’, ‘OCB-051’, or ‘CB15’, wherein representative seed of said variety have been deposited under ATCC Accession No. ______, respectively.

8. A seed mixture, comprising the seed of claim 7.

9. A composition comprising the seed of claim 7 and plant seed growth media.

10. The composition of claim 9, wherein the plant seed growth media is soil or a synthetic cultivation medium.

11. A method of producing basil seed, comprising crossing the plant of claim 1 with itself or a second basil plant.

12. The method of claim 11, wherein the second basil plant is transgenic.

13. An F1 sweet basil seed produced by the method of claim 11.

14. A basil plant or part thereof produced by growing the seed of claim 13, wherein the part thereof is pollen, a meristem, a cell, an ovule, a leaf, a root, a root tip, a pistil, an anther, a protoplast, or a cotyledon.

15. A plant of sweet basil variety ‘OCB-029’, ‘OCB-017’, ‘OCB-022’, ‘OCB-009’, ‘OCB-011’, ‘OCB-027’, ‘OCB-019’, ‘OCB-031’, ‘OCB-121’, ‘OCB-174’, ‘OCB-169’, ‘OCB-153’, ‘OCB-115’, ‘OCB-120’, ‘OCB-119’, ‘OCB-067’, ‘OCB-058’, ‘OCB-086’, ‘OCB-062’, ‘OCB-117’, ‘OCB-057’, ‘OCB-126’, ‘OCB-188’, ‘OCB-129’, ‘OCB-051’, or ‘CB15’, wherein representative seed of said variety have been deposited under American Type Culture Collection (ATCC) Accession No. ______, respectively, further comprising a single locus conversion introduced by backcrossing or transformation.

16. The plant of claim 15, wherein the single locus conversion comprises a transgene.

17. A seed that produces the plant of claim 15.

18. The seed of claim 17, wherein the single locus confers a trait selected from the group consisting of male sterility, herbicide tolerance, insect resistance, pest resistance, disease resistance, modified fatty acid metabolism, abiotic stress resistance, altered seed amino acid composition, site-specific genetic recombination, and modified carbohydrate metabolism.

19. The method of claim 11, wherein the method further comprises:
(a) crossing a plant grown from said basil seed with itself or a different sweet basil plant to produce a seed of a progeny plant of a subsequent generation;
(b) growing a progeny plant of a subsequent generation from said seed of a progeny plant of a subsequent generation and crossing the progeny plant of a subsequent generation with itself or a second plant to produce a progeny plant of a further subsequent generation; and
(c) repeating steps (a) and (b) using said progeny plant of a further subsequent generation from step (b) in place of the plant grown from said sweet basil seed in step (a), wherein steps (a) and (b) are repeated with sufficient inbreeding to produce an inbred sweet basil plant derived from the basil variety ‘OCB-029’, ‘OCB-017’, ‘OCB-022’, ‘OCB-009’, ‘OCB-011’, ‘OCB-027’, ‘OCB-019’, ‘OCB-031’, ‘OCB-121’, ‘OCB-174’, ‘OCB-169’, ‘OCB-153’, ‘OCB-115’, ‘OCB-120’, ‘OCB-119’, ‘OCB-067’, ‘OCB-058’, ‘OCB-086’, ‘OCB-062’, ‘OCB-117’, ‘OCB-057’, ‘OCB-126’, ‘OCB-188’, ‘OCB-129’, ‘OCB-051’, or ‘CB15’.

20. The method of claim 19, further comprising crossing said inbred basil plant derived from the basil variety ‘OCB-029’, ‘OCB-017’, ‘OCB-022’, ‘OCB-009’, ‘OCB-011’, ‘OCB-027’, ‘OCB-019’, ‘OCB-031’, ‘OCB-121’, ‘OCB-174’, ‘OCB-169’, ‘OCB-153’, ‘OCB-115’, ‘OCB-120’, ‘OCB-119’, ‘OCB-067’, ‘OCB-058’, ‘OCB-086’, ‘OCB-062’, ‘OCB-117’, ‘OCB-057’, ‘OCB-126’, ‘OCB-188’, ‘OCB-129’, ‘OCB-051’, or ‘CB15’ with a plant of a different genotype to produce a seed of a hybrid basil plant derived from the basil variety ‘OCB-029’, ‘OCB-017’, ‘OCB-022’, ‘OCB-009’, ‘OCB-011’, ‘OCB-027’, ‘OCB-019’, ‘OCB-031’, ‘OCB-121’, ‘OCB-174’, ‘OCB-169’, ‘OCB-153’, ‘OCB-115’, ‘OCB-120’, ‘OCB-119’, ‘OCB-067’, ‘OCB-058’, ‘OCB-086’, ‘OCB-062’, ‘OCB-117’, ‘OCB-057’, ‘OCB-126’, ‘OCB-188’, ‘OCB-129’, ‘OCB-051’, or ‘CB15’.

21. A method of producing a commodity plant product, the method comprising collecting or producing the commodity plant product from the plant of claim 1 or a part of the plant.

22. The method of claim 21, wherein the commodity plant product comprises a protein concentrate, protein isolate, biomass, leaves, extract, or oil.

23. A sweet basil commodity plant product produced by the method of claim 21, wherein the commodity plant product comprises at least one cell of and/or the genomic DNA of basil variety ‘OCB-029’, ‘OCB-017’, ‘OCB-022’, ‘OCB-009’, ‘OCB-011’, ‘OCB-027’, ‘OCB-019’, ‘OCB-031’, ‘OCB-121’, ‘OCB-174’, ‘OCB-169’, ‘OCB-153’, ‘OCB-115’, ‘OCB-120’, ‘OCB-119’, ‘OCB-067’, ‘OCB-058’, ‘OCB-086’, ‘OCB-062’, ‘OCB-117’, ‘OCB-057’, ‘OCB-126’, ‘OCB-188’, ‘OCB-129’, ‘OCB-051’, or ‘CB15’.

24. An extract or oil product of the plant of claim 1 or a part of the plant, wherein the extract or oil product comprises at least one cell of and/or the genomic DNA of basil variety ‘OCB-029’, ‘OCB-017’, ‘OCB-022’, ‘OCB-009’, ‘OCB-011’, ‘OCB-027’, ‘OCB-019’, ‘OCB-031’, ‘OCB-121’, ‘OCB-174’, ‘OCB-169’, ‘OCB-153’, ‘OCB-115’, ‘OCB-120’, ‘OCB-119’, ‘OCB-067’, ‘OCB-058’, ‘OCB-086’, ‘OCB-062’, ‘OCB-117’, ‘OCB-057’, ‘OCB-126’, ‘OCB-188’, ‘OCB-129’, ‘OCB-051’, or ‘CB15’.

25. A basil plant produced by transforming the basil plant of claim 1 with a transgene or editing an existing gene that confers upon the basil plant to a desired trait, wherein the desired trait is one or more of herbicide tolerance, drought tolerance, heat tolerance, low or high soil pH level tolerance, salt tolerance, resistance to an insect, resistance to a bacterial disease, resistance to a viral disease, resistance to a fungal disease, resistance to a nematode, resistance to a pest, male sterility, site-specific recombination; abiotic stress tolerance, modified phosphorus characteristics, modified antioxidant characteristics; modified essential seed amino acid characteristics, decreased phytate, modified fatty acid metabolism, modified carbohydrate metabolism, extended shelf life, flowering time and ornamental traits such as color, leaf size, and internode length.

26. A basil seed produced by crossing two basil plants and harvesting the resultant basil seed, wherein at least one of the two basil plants is the basil plant of claim 1.

27. A container, comprising
dried, frozen, and/or fresh leaves of the plant of claim 1.

28. A nanoparticle, comprising
dried, frozen, and/or fresh leaves of the plant of claim 1.

29. A method of producing a new sweet basil cultivar, comprising selecting a somaclonal variant of the plant of claim 1.

30. A method of producing a new sweet basil cultivar, comprising selecting a somaclonal variant of the of the tissue culture of claim 4.

31. A method of generating a basil plant having chilling tolerance, comprising:
crossing basil variety ‘CB15’ with a second basil variety, thereby generating a basil plant having chilling tolerance, wherein a representative sample of seed of the variety ‘CB15’ has been deposited under ATCC Accession No. ______.
